(19) United States
(12) Patent Application Publication
(10) Pub. No.: US 2004/0077853 A1
(43) Pub. Date: Apr. 22, 2004

(54) 2-amino-6-(2,4,5-substituted-phenyl)-pyridines

(75) Inventors: John A. Lowe III, Stonington, CT (US); Robert A. Volkmann, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/368,183

(22) Filed: Feb. 18, 2003

Related U.S. Application Data
(63) Continuation-in-part of application No. 10/266,249, filed on Oct. 8, 2002.
(60) Provisional application No. 60/328,252, filed on Oct. 10, 2001.
(60) Provisional application No. 60/328,253, filed on Oct. 10, 2001.

(51) Int. Cl. C07D 417/02; C07D 413/02; C 07D 4 3/02; C 07D 4 1/02; C07D 213/72

(52) U.S. Cl. 544/60; 544/124; 544/360; 546/269.7; 546/271.4; 546/272.7; 546/304

(57) ABSTRACT

The invention provides compounds of formula VI

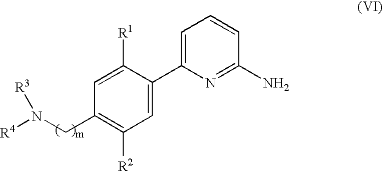

and the pharmaceutically acceptable salts thereof, wherein R1, R2, R3, and R4 are as defined, to pharmaceutical compositions containing such compounds and to the use of such compounds in the treatment and prevention of central nervous system and other disorders. The invention also provides methods for inhibiting neurological damage caused by impairment of glucose and/or oxygen to the brain in a mammal, which method comprises administering to the mammal a NOS inhibitor. In one embodiment, the NOS inhibitor is administered to the mammal prior to surgery, for example prior to cardiac surgery, angioplasty, or angiography.

This application claims priority under 35 USC 120 of U.S. Ser. No. 10/266,249, filed Oct. 8, 2002.

The present invention relates to certain 2-amino-6-(2,4,5-substituted-phenyl)-pyridines, to pharmaceutical compositions containing them and to their use in the treatment and prevention of central nervous system and other disorders The compounds of this invention exhibit activity as nitric oxide synthase (NOS) inhibitors.

There are three known isoforms of NOS—an inducible form (I-NOS) and two constitutive forms referred to as, respectively, neuronal NOS(N-NOS) and endothelial NOS (E-NOS). Each of these enzymes carries out the conversion of arginine to citrulline while producing a molecule of nitric oxide (NO) in response to various stimuli. It is believed that excess nitric oxide (NO) production by NOS plays a role in the pathology of a number of disorders and conditions in mammals. For example, NO produced by I-NOS is thought to play a role in diseases that involve systemic hypotension such as toxic shock and therapy with certain cytokines. It has been shown that cancer patients treated with cytokines such as interleukin 1 (IL-1), interleukin 2 (IL-2) or tumor necrosis factor (TNF) suffer cytokine-induced shock and hypotension due to NO produced from macrophages, i.e., inducible NOS (I-NOS), see Chemical & Engineering News, December 20, p. 33, (1993). I-NOS inhibitors can reverse this. It is also believed that I-NOS plays a role in the pathology of diseases of the central nervous system such as ischemia. For example, inhibition of I-NOS has been shown to ameliorate cerebral ischemic damage in rats, see Am. J. Physiol., 268, p. R286 (1995)). Suppression of adjuvant induced arthritis by selective inhibition of I-NOS is reported in Eur. J. Pharmacol., 273, p. 15-24 (1995).

NO produced by N-NOS is thought to play a role in diseases such as cerebral ischemia, pain, and opiate tolerance. For example, inhibition of N-NOS decreases infarct volume after proximal middle cerebral artery occlusion in the rat, see J. Cerebr. Blood Flow Metab., 14, p. 924-929 (1994). N-NOS inhibition has also been shown to be effective in antinociception, as evidenced by activity in the late phase of the formalin-induced hindpaw licking and acetic acid-induced abdominal constriction assays, see Br. J. Pharmacol., 110, p. 219-224 (1993). Finally, opioid withdrawal in rodents has been reported to be reduced by N-NOS inhibition, see Neuropsychopharmacol., 13, p. 269-293 (1995).

Other NOS inhibitors and their utility as pharmaceutical agents in the treatment of central nervous system disorders and other disorders are referred to in the following references: U.S. patent application Ser. No. 09/325,480, filed Jun. 3, 1999, allowed Nov. 14, 2000, U.S. patent application Ser. No. 09/802,086, filed Mar. 8, 2001, and counterpart International Patent Application No. WO 98/24766, published Jun. 11, 1998; U.S. Pat. No. 6,235,747, issued May 22, 2001, U.S. patent application Ser. No. 09/826,132, filed Apr. 4, 2001, and counterpart International Patent Application No. WO 97/36871, published Oct. 9, 1997; U.S. patent application Ser. No. 09/740,385, filed Dec. 20, 2000, and counterpart International Patent Application No. WO 99/10339, published Mar. 4, 1999; U.S. patent application Ser. No. 09/381,887, filed Mar. 28, 2000, and counterpart International Patent Application No. WO 99/11620, published Mar. 11, 1999; U.S. patent application Ser. No. 09/127,158, filed Jul. 31, 1998, and counterpart International Patent Application No. WO 98/34919, published Aug. 13, 1998; and U.S. patent application Ser. No. 09/403,177, filed Oct. 18, 1999, and counterpart International Patent Application No. WO 99/62883, published Dec. 9, 1999.

### SUMMARY OF THE INVENTION

The present invention relates to a compound, or pharmaceutically acceptable salt thereof, that is selected from the following compounds and their pharmaceutically acceptable salts:

(a) 6-[4-(N-methyl-3-azetidinoxy)-5-ethyl-2-methoxy-phenyl]-pyridin-2-ylamine, which has the following structure

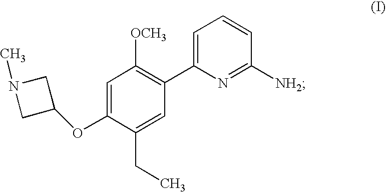

(b) 6-[4-(N,N-dimethylaminomethyl)-5-ethyl-2-methoxy-phenyl]-pyridin-2-ylamine, which has the following structure

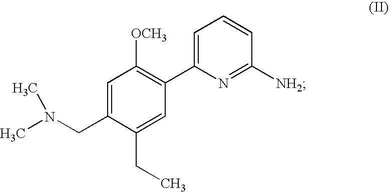

(c) 6-[4-(N-methylaminomethyl)-5-ethyl-2-methoxy-phenyl]-pyridin-2-ylamine, which has the following structure

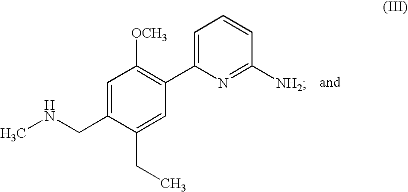

(d) 6-[4-(3-azetidinoxy)-5-ethyl-2-methoxy-phenyl]-pyridin-2-ylamine, which has the following structure

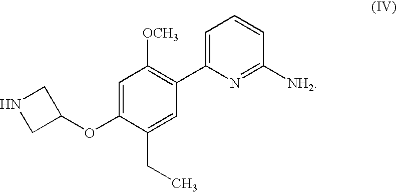

The present invention also relates to compounds of formula VI

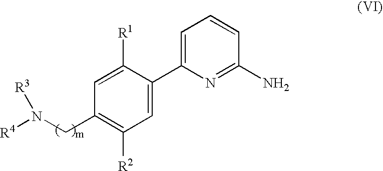

wherein R¹ is selected from methyl, ethyl, propyl, butyl, isopropyl, 2-methylpropyl, t-butyl, methoxy, ethoxy, and propoxy;

R² is selected from hydrogen, methyl, ethyl, propyl, butyl, isopropyl, 1-methylpropyl, 2-methylpropyl, t-butyl, methoxy, ethoxy, and propoxy;

m is one, two or three;

R³ and R⁴ are selected, independently, from R⁷; phenyl; 5 or 6 membered heteroaryl containing from 1 to 4 heteroatoms independently selected from O, N, and S; and straight chain or branched (C₁-C₆) alkyl substituted with from 1 to 3 substituents selected independently from R⁶, —CF₃, halo, (i.e. bromine, chlorine, iodine, and fluorine), —NR⁷R⁸, (C3-C6) cycloalkyl, 3 to 9 membered heterocycloalkyl containing 1 or 2 heteroatoms independently selected from O, N, and S, phenyl, and 5 or 6 membered heteroaryl containing from 1 to 4 heteroatoms independently selected from O, N, and S;

wherein said phenyl, heteroaryl, cycloalkyl, and heterocycloalkyl groups of R³ and R⁴ are optionally independently substituted with from 1 to 3 substituents independently selected from R⁶ and straight chain or branched C₁-C₆ alkyl optionally comprising 1 or 2 double or triple bonds;

or R³ and R⁴ are connected, with the nitrogen atom to which they are attached, to form a 3 to 9 membered heterocyclic ring, which heterocyclic optionally comprises from one to three heteroatoms in addition to said nitrogen atom, which optional heteroatoms are selected independently from O, S, and N;

wherein said heterocyclic ring formed by R³ and R⁴ optionally is fused to form a fused ring system with one or two aromatic rings selected independently from benzene rings and heteroaromatic rings, which aromatic rings share two carbon atoms with said heterocyclic ring; or which heterocyclic ring formed by R³ and R⁴ is optionally fused to form a fused or spiro ring system to a 3 to 8 membered carbocyclic ring which shares one or two carbon atoms with said heterocyclic ring; wherein fused or spiro ring systems contain up to 15 ring members;

and wherein said heterocyclic ring, said optional aromatic rings, and said optional carbocyclic ring, are each optionally and independently substituted with from 1 to 3 substituents independently selected from R⁶, —O—(C₁-C₆ alkyl)-R⁶, —S—(C₁-C₆ alkyl)-R⁶, straight chain or branched (C₁-C₆) alkyl optionally substituted with R⁶, —C(═O)O—((C₁-C₆) alkyl), 3 to 6 membered cycloalkyl, phenyl, benzyl, and 5 or 6 membered heteroaryl; wherein said cycloalkyl, phenyl, benzyl, and heteroaryl are independently optionally substituted with from 1 to 3 substituents independently selected from R⁵;

R⁵ is selected from R⁶, straight chain or branched (C₁-C₆ alkyl), —(C₁-C₆ alkyl)-R⁶, and 5 or 6 membered heteroaryl optionally substituted with 1 or 2 substituents independently selected from R⁶, —NR⁷R⁸, straight chain or branched (C₁-C₆) alkyl, and (C₁-C₆) alkyl-R⁶;

R⁶ is selected from —O—R⁷ and —S—R⁷;

R⁷ is selected from H and straight chain or branched (C₁-C₆) alkyl (e.g. methyl, ethyl, propyl, butyl, isopropyl, 1-methylpropyl, 2-methylpropyl, t-butyl, pentyl, 3-methylbutyl, 1,2-dimethylpropyl, or 1,1-dimethylbutyl) optionally comprising 1 or 2 double or triple bonds; and

R⁸ is selected from H and straight chain or branched (C₁-C₆) alkyl;

and to pharmaceutically acceptable salts thereof.

In one embodiment, this invention provides compounds of formula VI and their pharmaceutically acceptable salts wherein m is 1. In another embodiment, m is 2. In another embodiment, m is 3.

In another embodiment, this invention provides compounds of formula VI and their pharmaceutically acceptable salts wherein R³ and R⁴ are selected from H and methyl. In another embodiment, R³ and R⁴ are both methyl. In another embodiment one of R³ and R⁴ is methyl, and the other of R³ and R⁴ is H. The compounds of formula II and III of this invention are preferred examples of such compounds of formula VI wherein R³ and R⁴ are selected from H and methyl.

In another embodiment, this invention provides compounds of formula VI and their pharmaceutically acceptable salts wherein R¹ is selected from methyl, ethyl, and methoxy, and R² is selected from ethyl and methoxy. In one embodiment, R¹ is methoxy. In another embodiment R¹ and R² are both methoxy. In another embodiment, R¹ is methoxy and R² is ethyl.

In another embodiment, this invention provides compounds of formula VI, and their pharmaceutically acceptable salts, as defined above, with the proviso that when R¹ is —OCH₃, R² is ethyl, and R⁴ is methyl, then R³ is not hydrogen or methyl.

In so far as the compounds of formulas I, II, III, IV, and VI of this invention contain basic groups, they can form acid addition salts with various inorganic and organic acids. The present invention also relates to the pharmaceutically acceptable acid addition salts of compounds of the formulas I, II, III, IV, and VI. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent, and thereafter, convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate))salts.

The present invention also includes isotopically-labeled compounds that are identical to those recited in formulas I, II, III, IV, and VI but for the fact that one or more atoms are replaced by an atom having an atomic mass or mass number different from the atomic mass or mass number usually found in nature. Examples of isotopes that can be incorporated into the compounds of the present invention include isotopes of hydrogen, carbon, nitrogen and oxygen, such as ²H, ³H, ¹³C, ¹¹C, ¹⁴C, ¹⁵N, ¹⁸O, ¹⁷O, respectively. The compounds of the present invention, prodrugs thereof, and pharmaceutically acceptable salts of such compounds or of such prodrugs which contain the aforementioned isotopes and/or other isotopes are within the scope of this invention. Such compounds may be useful as research and diagnostic tools in metabolism pharmacokinetic studies and in binding assays. Certain isotopically-labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of the present invention and prodrugs thereof can generally be prepared by carrying out the procedures disclosed in the schemes and discussion of the schemes and/or in the examples and preparations described herein, by substituting a readily available isotopically-labeled reagent for a nonisotopically-labeled reagent.

More specific embodiments of this invention relate to a compound of the formula I, which has the chemical name 6-[4-(N-methyl-3-azetidinoxy)-5-ethyl-2-methoxy-phenyl]-pyridin-2-ylamine, and the pharmaceutically acceptable salts of such compound.

Other more specific embodiments of this invention relate to a compound of the formula II, which has the chemical name 6-[4-(N,N-dimethylaminomethyl)-5-ethyl-2-methoxy-phenyl]-pyridin-2-ylamine, and the pharmaceutically acceptable salts of such compound.

Other more specific embodiments of this invention relate to a compound of the formula III, which has the chemical name 6-[4-(N-methylaminomethyl)-5-ethyl-2-methoxy-phenyl]-pyridin-2-ylamine, and the pharmaceutically acceptable salts of such compound.

Other more specific embodiments of this invention relate to a compound of the formula IV, which has the chemical name 6-[4-(3-azetidinoxy)-5-ethyl-2-methoxy-phenyl]-pyridin-2-ylamine, and the pharmaceutically acceptable salts of such compound.

The compounds of formulas I, II, III, IV, and VI of this invention, and their pharmaceutically acceptable salts, have useful pharmaceutical and medicinal properties. The compounds of formulas I, II, III, IV, and VI, and their pharmaceutically acceptable salts, are useful as NOS inhibitors i.e., they possess the ability to inhibit the NOS enzyme in mammals, and therefore they are able to function as therapeutic agents in the treatment of the disorders and diseases enumerated below in an afflicted mammal.

The term “treating,” as used herein, refers to reversing, alleviating, or inhibiting the progress of the disease, disorder or condition, or one or more symptoms of such disease, disorder or condition, to which such term applies. Depending on the condition of the patient, as used herein, this term also refers to preventing a disease, disorder or condition, and includes preventing the onset of a disease, disorder or condition, or preventing the symptoms associated with a disease, disorder or condition. As used herein, this term also refers to reducing the severity of a disease, disorder or condition or symptoms associated with such disease, disorder or condition prior to affliction with the disease, disorder or condition. Such prevention or reduction of the severity of a disease, disorder or condition prior to affliction refers to administration of the composition of the present invention, as described herein, to a subject that is not at the time of administration afflicted with the disease, disorder or condition. “Preventing” also refers to preventing the recurrence of a disease, disorder or condition or of one or more symptoms associated with such disease, disorder or condition. The terms “treatment” and “therapeutically,” as used herein, refer to the act of treating, as “treating” is defined above.

The present invention also relates to a pharmaceutical composition for treating a condition selected from the group consisting of migraine, inflammatory diseases (e.g., asthma, psoriasis, eczema, arthritis), stroke, acute, chronic and neuropathic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addiction (e.g., dependencies on drugs, alcohol and nicotine), emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, ovulation, dilated cardiomyopathy, acute spinal cord injury, Huntington's disease, Parkinson's disease, glaucoma, macular degeneration, diabetic neuropathy, diabetic nephropathy and cancer in a mammal, including a human, comprising an amount of a compound of the formula I, II, III, IV, or VI, or a pharmaceutically acceptable salt thereof, that is effective in treating such condition, and a pharmaceutically acceptable carrier.

This invention also relates to the above pharmaceutical composition for treating a condition selected from the group of conditions referred to above, in a mammal, including a human, comprising an amount of a compound of the formula I, or a pharmaceutically acceptable salt thereof, that is effective in treating such condition, and a pharmaceutically acceptable carrier.

This invention also relates to the above pharmaceutical composition for treating a condition selected from the group of conditions referred to above, in a mammal, including a human, comprising an amount of a compound of the formula II, or a pharmaceutically acceptable salt thereof, that is effective in treating such condition, and a pharmaceutically acceptable carrier.

This invention also relates to the above pharmaceutical composition for treating a condition selected from the group of conditions referred to above, in a mammal, including a human, comprising an amount of a compound of the formula II, or a pharmaceutically acceptable salt thereof, that is effective in treating such condition, and a pharmaceutically acceptable carrier.

This invention also relates to the above pharmaceutical composition for treating a condition selected from the group of conditions referred to above, in a mammal, including a human, comprising an amount of a compound of the formula IV, or a pharmaceutically acceptable salt thereof, that is effective in treating such condition, and a pharmaceutically acceptable carrier.

This invention also relates to the above pharmaceutical composition for treating a condition selected from the group of conditions referred to above, in a mammal, including a human, comprising an amount of a compound of the formula VI, or a pharmaceutically acceptable salt thereof, that is effective in treating such condition, and a pharmaceutically acceptable carrier.

The present invention also relates to a method of treating a condition selected from the group consisting of migraine, inflammatory diseases (e.g., asthma, psoriasis, eczema, arthritis), stroke, acute, chronic and neuropathic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions (e.g., dependencies on drugs, alcohol and nicotine), emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, ovulation, dilated cardiomyopathy, acute spinal cord injury, Huntington's disease, Parkinson's disease, glaucoma, macular degeneration, diabetic neuropathy, diabetic nephropathy and cancer in a mammal, including a human, comprising administering to said mammal an amount of a compound of the formula I, II, III, IV, or VI, or a pharmaceutically acceptable salt thereof, that is effective in treating such condition.

This invention also relates to the above method of treating a condition selected from the group of conditions referred to above, in a mammal, including a human, comprising administering to said mammal an amount of a compound of the formula I, or a pharmaceutically acceptable salt thereof, that is effective in treating such condition.

This invention also relates to the above method of treating a condition selected from the group of conditions referred to above, in a mammal, including a human, comprising administering to said mammal an amount of a compound of the formula II, or a pharmaceutically acceptable salt thereof, that is effective in treating such condition.

This invention also relates to the above method of treating a condition selected from the group of conditions referred to above, in a mammal, including a human, comprising administering to said mammal an amount of a compound of the formula II, or a pharmaceutically acceptable salt thereof, that is effective in treating such condition.

This invention also relates to the above method of treating a condition selected from the group of conditions referred to above, in a mammal, including a human, comprising administering to said mammal an amount of a compound of the formula IV, or a pharmaceutically acceptable salt thereof, that is effective in treating such condition.

This invention also relates to the above method of treating a condition selected from the group of conditions referred to above, in a mammal, including a human, comprising administering to said mammal an amount of a compound of the formula VI, or a pharmaceutically acceptable salt thereof, that is effective in treating such condition.

The present invention also relates to a pharmaceutical composition for inhibiting nitric oxide synthase (NOS) in a mammal, including a human, comprising a NOS inhibiting effective amount of a compound of the formula I, II, III, IV, or VI, or a pharmaceutically acceptable salt thereof, and a pharmaceutically acceptable carrier.

This invention also relates to the above pharmaceutical composition for inhibiting NOS in a mammal, including a human, comprising a NOS inhibiting effective amount of a compound of the formula I, or a pharmaceutically acceptable salt thereof, and a pharmaceutically acceptable carrier.

This invention also relates to the above pharmaceutical composition for inhibiting NOS in a mammal, including a human, comprising a NOS inhibiting effective amount of a compound of the formula II, or a pharmaceutically acceptable salt thereof, and a pharmaceutically acceptable carrier.

This invention also relates to the above pharmaceutical composition for inhibiting NOS in a mammal, including a human, comprising a NOS inhibiting effective amount of a compound of the formula II, or a pharmaceutically acceptable salt thereof, and a pharmaceutically acceptable carrier.

This invention also relates to the above pharmaceutical composition for inhibiting NOS in a mammal, including a human, comprising a NOS inhibiting effective amount of a compound of the formula IV, or a pharmaceutically acceptable salt thereof, and a pharmaceutically acceptable carrier.

This invention also relates to the above pharmaceutical composition for inhibiting NOS in a mammal, including a human, comprising a NOS inhibiting effective amount of a compound of the formula VI, or a pharmaceutically acceptable salt thereof, and a pharmaceutically acceptable carrier.

The present invention also relates to a method of inhibiting NOS in a mammal, including a human, comprising administering to said mammal a NOS inhibiting effective amount of a compound of the formula I, II, III, IV, or VI, or a pharmaceutically acceptable salt thereof.

This invention also relates to the above method of inhibiting NOS in a mammal, including a human, comprising administering to said mammal a NOS inhibiting effective amount of a compound of the formula I, or a pharmaceutically acceptable salt thereof.

This invention also relates to the above method of inhibiting NOS in a mammal, including a human, comprising administering to said mammal a NOS inhibiting effective amount of a compound of the formula II, or a pharmaceutically acceptable salt thereof.

This invention also relates to the above method of inhibiting NOS in a mammal, including a human, comprising administering to said mammal a NOS inhibiting effective amount of a compound of the formula III, or a pharmaceutically acceptable salt thereof.

This invention also relates to the above method of inhibiting NOS in a mammal, including a human, comprising administering to said mammal a NOS inhibiting effective amount of a compound of the formula IV, or a pharmaceutically acceptable salt thereof.

This invention also relates to the above method of inhibiting NOS in a mammal, including a human, comprising administering to said mammal a NOS inhibiting effective amount of a compound of the formula VI, or a pharmaceutically acceptable salt thereof.

The present invention also relates to a pharmaceutical composition for treating a condition selected from the group consisting of migraine, inflammatory diseases (e.g., asthma, psoriasis, arthritis, eczema), stroke, acute, chronic and neuropathic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions (e.g., dependencies on drugs, alcohol and nicotine), emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, ovulation, dilated cardiomyopathy, acute spinal cord injury, Huntington's disease, glaucoma, macular degeneration, diabetic neuropathy, diabetic nephropathy and cancer in a mammal, including a human, comprising a NOS inhibiting effective amount of a compound of the formula I, II, III, IV, or VI, or a pharmaceutically acceptable salt thereof, and a pharmaceutically acceptable carrier.

This invention also relates to the above pharmaceutical composition for treating a condition selected from the group of conditions referred to above, in a mammal, including a human, comprising a NOS inhibiting effective amount of a compound of the formula I, or a pharmaceutically acceptable salt thereof, and a pharmaceutically acceptable carrier.

This invention also relates to the above pharmaceutical composition for treating a condition selected from the group of conditions referred to above, in a mammal, including a human, comprising a NOS inhibiting effective amount of a compound of the formula II, or a pharmaceutically acceptable salt thereof, and a pharmaceutically acceptable carrier.

This invention also relates to the above pharmaceutical composition for treating a condition selected from the group of conditions referred to above, in a mammal, including a human, comprising a NOS inhibiting effective amount of a compound of the formula III, or a pharmaceutically acceptable salt thereof, and a pharmaceutically acceptable carrier.

This invention also relates to the above pharmaceutical composition for treating a condition selected from the group of conditions referred to above, in a mammal, including a human, comprising a NOS inhibiting effective amount of a compound of the formula IV, or a pharmaceutically acceptable salt thereof, and a pharmaceutically acceptable carrier.

This invention also relates to the above pharmaceutical composition for treating a condition selected from the group of conditions referred to above, in a mammal, including a human, comprising a NOS inhibiting effective amount of a compound of the formula VI, or a pharmaceutically acceptable salt thereof, and a pharmaceutically acceptable carrier.

The present invention also relates to a method of treating a condition selected from the group consisting of migraine, inflammatory diseases (e.g., asthma, psoriasis, eczema, arthritis), stroke, acute, chronic and neuropathic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions (e.g., dependencies on drugs, alcohol or nicotine), emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, ovulation, dilated cardiomyopathy, acute spinal cord injury, Huntington's disease, Parkinson's disease, glaucoma, macular degeneration, diabetic neuropathy, diabetic nephropathy and cancer in a mammal, including a human, comprising administering to said mammal a NOS inhibiting effective amount of a compound of the formula I, II, III, IV, or VI, or a pharmaceutically acceptable salt thereof.

This invention also relates to the above method of treating a condition selected from the group of conditions referred to above, in a mammal, including a human, comprising administering to said mammal a NOS inhibiting effective amount of a compound of the formula I, or a pharmaceutically acceptable salt thereof.

This invention also relates to the above method of treating a condition selected from the group of conditions referred to above, in a mammal, including a human, comprising administering to said mammal a NOS inhibiting effective amount of a compound of the formula II, or a pharmaceutically acceptable salt thereof.

This invention also relates to the above method of treating a condition selected from the group of conditions referred to above, in a mammal, including a human, comprising administering to said mammal a NOS inhibiting effective amount of a compound of the formula III, or a pharmaceutically acceptable salt thereof.

This invention also relates to the above method of treating a condition selected from the group of conditions referred to above, in a mammal, including a human, comprising administering to said mammal a NOS inhibiting effective amount of a compound of the formula IV, or a pharmaceutically acceptable salt thereof.

This invention also relates to the above method of treating a condition selected from the group of conditions referred to above, in a mammal, including a human, comprising administering to said mammal a NOS inhibiting effective amount of a compound of the formula VI, or a pharmaceutically acceptable salt thereof.

The present invention also relates to prophylactic use of a NOS inhibitor to inhibit neurological damage following an impairment of glucose and/or oxygen supply to or in the brain. Accordingly, this invention also provides a method of inhibiting neurological damage caused by impairment of glucose and/or oxygen to the brain in a mammal, including a human, which method comprises administering to the mammal an amount of a NOS inhibitor, which amount is effective in inhibiting neurological damage. In one embodiment, the NOS inhibitor is administered to the mammal prior to an event having associated therewith risk of impairment of glucose and/or oxygen supply to the brain.

“Inhibiting neurological damage” means a reduction of neurological damage following impairment of glucose and/or oxygen supply to or in the brain compared to the neurological damage that would otherwise have occurred had the NMDA antagonist not been administered.

Neurological damage that is “caused by” impairment of glucose and/or oxygen supply is neurological damage caused at least in part by an insufficiency in the level of glucose and/or oxygen in the brain.

In one embodiment of the method of inhibiting neurological damage described above, the NOS inhibitor is administered to the mammal prior to an event having associated therewith risk of impairment of glucose and/or oxygen supply to the brain, such as an event wherein there exists risk of hypoxia, anoxia, asphyxia, or brain ischemia.

In another embodiment of the method of inhibiting neurological damage described above, the mammal to whom the NOS inhibitor is administered is a mammal predisposed to or at risk of brain ischemia, for example stroke.

Examples of events having associated therewith risk of brain ischemia include surgeries, especially surgeries pertaining to the lungs, the cardiovascular system (particularly the cerebrovascular system), or the central nervous system. However, any type of surgery carries with it a risk of brain ischemia. One specific example of a type of surgery wherein the risk of ischemic injury is relatively high is a coronary artery bypass graft (CABG). Other examples are cardiac surgery (for example heart surgery), angiography, and angioplasty. Patients undergoing CABG or other surgeries that have associated therewith a high risk of brain ischemia can benefit from NOS inhibitor.

Other events wherein oxygen supply to the brain may be impaired are events wherein there is a risk of hypoxia, anoxia, or asphyxia. It is thus also beneficial to administer to a mammal, according to the present invention, a NOS inhibitor prior to an event wherein there is a risk of hypoxia, anoxia, or perinatal asphyxia.

Other examples wherein risk of glucose and/or oxygen impairment to or in the brain may be predicted or likely are in patients predisposed to or at risk of brain ischemia, for example stroke. If, for example, a patient has suffered a prior stroke, or has suffered a cardiovascular disease or other condition that impairs the cardiovascular system, that patient may be determined to be predisposed to or at risk of brain ischemia such as stroke. Examples of cardiovascular diseases or other conditions that can impair the cardiovascular system include, but are not limited to, heart-failure, atrial fibrillation, cardiac ischemia, a hypercoagulative state, birth-control pill use, estrogen replacement therapy, poor circulation, atherosclerosis, or congestive heart failure.

In the method of this invention of inhibiting neurological damage resulting from impairment of glucose and/or oxygen supply to or in the brain, the NOS inhibitor is preferably administered prior to the event, for example “surgery”, comprising a risk of impairment of glucose and/or oxygen to or in the brain, for example a risk of brain ischemia. Or, as another example, the NOS inhibitor is administered prior to an event wherein there exists a risk of hypoxia, anoxia, or perinatal asphyxia.

Examples of NOS inhibitors that can be used in the present method of inhibiting neurological damage caused by impairment of glucose and/or oxygen include, but are not limited to, compounds of formula I, II, III, IV, and VI, and pharmaceutically acceptable salts thereof, as described herein.

Another example of a NOS inhibitor that can be used, as well as its pharmaceutically acceptable salts, in the present method of inhibiting neurological damage caused by impairment of glucose and/or oxygen is the compound 6-[4-(2-dimethylamino-ethoxy)-5-ethyl-2-methoxy-phenyl]-pyridin-2-ylamine, which has the following structure

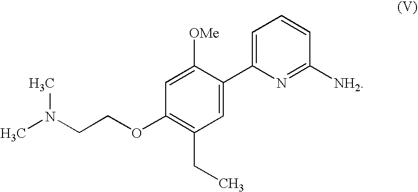

The compound of formula (V) is disclosed, and its synthesis described, in PCT/IB02//03939, which designates the United States, filed Sep. 24, 2002; and in U.S. Ser. No. 09/127,158, filed Jul. 31, 1998; both of which are incorporated by reference herein in their entireties.

Other examples of NOS inhibitors that can be used in the present method of inhibiting neurological damage caused by impairment of glucose and/or oxygen are compounds of the formula

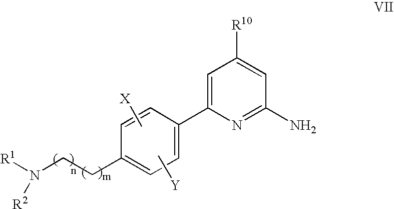

wherein R¹ and R² are selected, independently, from (C₁-C₆) alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₆) alkyl and said tetrahydronaphthalene and the aryl moiety of said aralkyl may optionally be substituted with from one to three substituents, preferably from zero to two substituents, that are selected, independently, from halo (e.g., chloro, fluoro, bromo, iodo), nitro, hydroxy, cyano, amino, (C₁-C₄) alkoxy, and (C₁-C₄) alkylamino;

or R¹ and R² form, together with the nitrogen to which they are attached, a piperazine, piperidine or pyrrolidine ring or an azabicyclic ring containing from 6 to 14 ring members, from 1 to 3 of which are nitrogen and the rest of which are carbon, wherein examples of said azabicyclic rings are the following

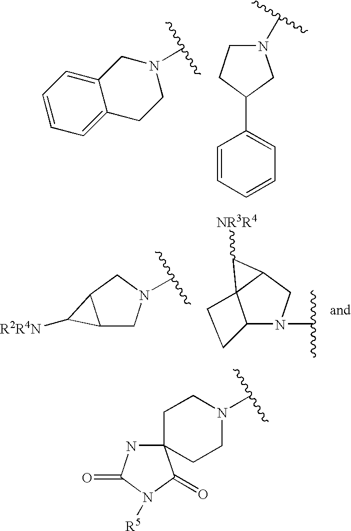

wherein R³ and R⁴ are selected from hydrogen, (C₁-C₆)alkyl, phenyl, naphthyl, (C₁-C₆)alkyl-C(═O)—, HC(═O)—, (C₁-C₆)alkoxy-(C═O)-, phenyl-C(═O)—, naphthyl-C(═O)—, and —(R⁷)₂NC(═O)— wherein each R⁷ is selected, independently, from hydrogen and (C₁-C₆)alkyl;

R⁵ is selected from hydrogen, (C₁-C₆)alkyl, phenyl, napthyl, phenyl-(C₁-C₆)alkyl- and naphthyl (C₁-C₆)alkyl-;

and wherein said piperazine, piperidine and pyrorrolidine rings may optionally be substituted with one or more substituents, preferably with from zero to two substituents, that selected independently, from (C₁-C₆) alkylamino, [di(C₁-C₆)alkyl]amino, pheynyl substituted 5 to 6 membered heterocyclic rings containing from 1 to 4 rings nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;

n is 0, 1 or 2; and each carbon of said (CH₂), can optionally be substituted with a substituent R⁸;

m is 0, 1, or 2; and each carbon of said (CH₂)ₘ can optionally be substituted with a substituent R⁹;

(C₁-C₄)alkyl, aryl-(C₁-C₄)alkyl wherein said aryl is selected from phenyl and naphthyl; allyl and phenallyl;

X and Y are selected, independently, from methyl, methoxy, hydroxy and hydrogen; and R¹⁰ is H(C₁-C₆)alkyl;

with the proviso that R⁸ is absent when n is zero and R⁹ is absent when m is zero.

Compounds of formula VII are disclosed, and their synthesis described, in U.S. Ser. No. 08/816,235, filed Mar. 13, 1997, now U.S. Pat. No. 6,235,747, issued May 22, 2001; and U.S. Ser. No. 09/826,132, filed Apr. 4, 2001, now U.S. Pat. No. 6,465,491, issued Oct. 15, 2002; both of which are incorporated herein by reference in their entireties.

Other NOS inhibitors that are useful in the methods and pharmaceutical compositions of the present invention are compounds of the formula

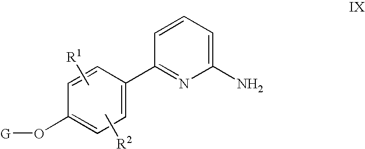

wherein R¹ and R² are selected, independently, from hydrogen, halo, hydroxy, (C₁-C₆)alkoxy, (C₁-C₇)alkyl, (C₂-C₆)alkenyl, and (C₂-C₁₋₀)alkoxyalkyl; and

G is selected from hydrogen, (C₁-C₆)alkyl, (C₁-C₆)alkoxy-(C₁-C₃)alkyl, aminocarbonyl-(C₁-C₃)alkyl-, (C₁-C₃) alkylaminocarbonyl-(C₁-C₃) alkyl-, di-[(C₁-C₃)alkyl]aminocarbonyl-(C₁-C₃)alkyl-, and N(R³)(R⁴)(C₀-C₄)alkyl-, wherein R³ and R⁴ are selected, independently, from hydrogen, (C₁-C₇) alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₇) alkyl and said tetrahydronaphthalene and the aryl moiety of said aralkyl may optionally be substituted with from one to three substituents, preferably from zero to two substituents, that are selected, independently, from halo, nitro, hydroxy, cyano, amino, (C₁-C₄) alkoxy, and (C₁-C₄) alkylamino;

or R³ and R⁴ form, together with the nitrogen to which they are attached, a piperazine, piperidine, azetidine or pyrrolidine ring or a saturated or unsaturated azabicyclic ring system containing from 6 to 14 ring members, from 1 to 3 of which are nitrogen, from zero to two of which are oxygen, and the rest of which are carbon;

and wherein said piperazine, piperidine, azetidine and pyrrolidine rings and said azabicyclic ring systems may optionally be substituted with one or more substituents, preferably with from zero to two substituents, that are selected, independently, from (C₁-C₆)alkyl, amino, (C₁-C₆) alkylamino, [di-(C₁-C₆)alkyl]amino, phenyl substituted 5 to 6 membered heterocyclic rings containing from 1 to 4 ring nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;

and wherein said piperazine, piperidine, azetidine and pyrrolidine rings and said azabicyclic ring systems may be attached to —(C₀-C₄)alkyl-O— (wherein the oxygen of said —(C₀-C₄)alkyl-O— is the oxygen atom depicted in structural formula I) at a nitrogen atom of the NR³R⁴ ring or at any other atom of such ring having an available bonding site;

or G is a group of the formula A

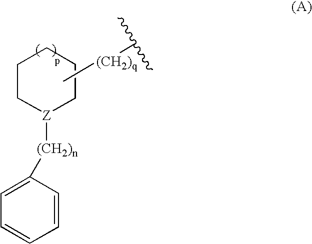

wherein Z is nitrogen or CH, n is zero or one, q is zero, one, two or three and p is zero, one or two;

and wherein the 2-amino piperidine ring depicted in structure I above may optionally be replaced with

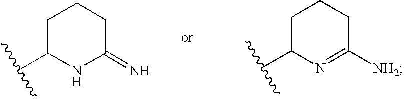

and the pharmaceutically acceptable salts of such compounds.

The compounds of formula IX are disclosed and their synthesis described in U.S. Ser. No. 09/127,158, mentioned and incorporated herein by reference above.

The present invention also provides a pharmaceutical composition for inhibiting neurological damage caused by impairment of glucose and/or oxygen to the brain in a mammal, including a human, which composition comprises a NOS inhibitor in an amount effective in inhibiting neurological damage and a pharmaceutically acceptable carrier. Examples of NOS inhibitors that can be used in such a pharmaceutical composition include, but are not limited to, compounds of formula I, II, III, IV, V, VI, VII, and IX, and pharmaceutically acceptable salts thereof, as described herein. In one embodiment, the pharmaceutical composition is a formulation that is suitable for administration prior to and during surgery, for example CABG surgery or another surgery pertaining to lungs, the cardiovascular system, or the central nervous system.

The present invention also provides a pharmaceutical composition for inhibiting neurological damage caused by impairment of glucose and/or oxygen to the brain in a mammal, including a human, which composition comprises a NOS inhibitor in an amount effective in inhibiting NOS and a pharmaceutically acceptable carrier. Examples of NOS inhibitors that can be used in such a pharmaceutical composition include, but are not limited to, compounds of formula I, II, III, IV, V, VI, VII, and IX, and pharmaceutically acceptable salts thereof, as described herein. In one embodiment, the pharmaceutical composition is a formulation that is suitable for administration prior to and during surgery, for example CABG surgery or another surgery pertaining to lungs, the cardiovascular system, or the central nervous system.

### DETAILED DESCRIPTION OF THE INVENTION

In the reaction Schemes and discussion that follow, formulas I, II, III, IV, and VI are defined as set forth above in the Summary of the Invention, unless otherwise indicated. DCE refers to “dichloroethane”. NBS refers to “N-bromo-succinimade”. TBDMS refers to “tertiary-butyl-dimethyl-silyl”.

Compounds of the formulas I and IV, and their pharmaceutically acceptable salts, may be prepared as described in the following reaction Schemes and discussion, and as described in U.S. patent application Ser. No. 09/127,158, filed Jul. 31, 1998, entitled 2-Amino-6-(2-substituted-4-phenoxy)-substituted-pyridines, and counterpart International Patent Application No. WO 98/34919, published Aug. 13, 1998. The foregoing patent applications are incorporated herein by reference in their entirety.

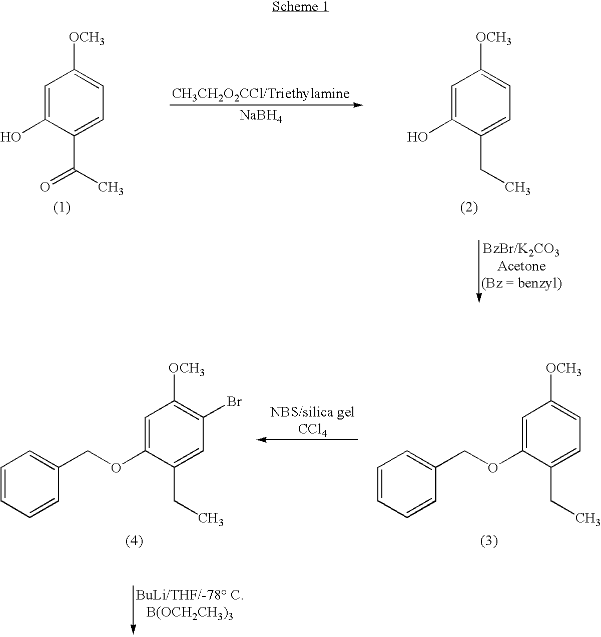

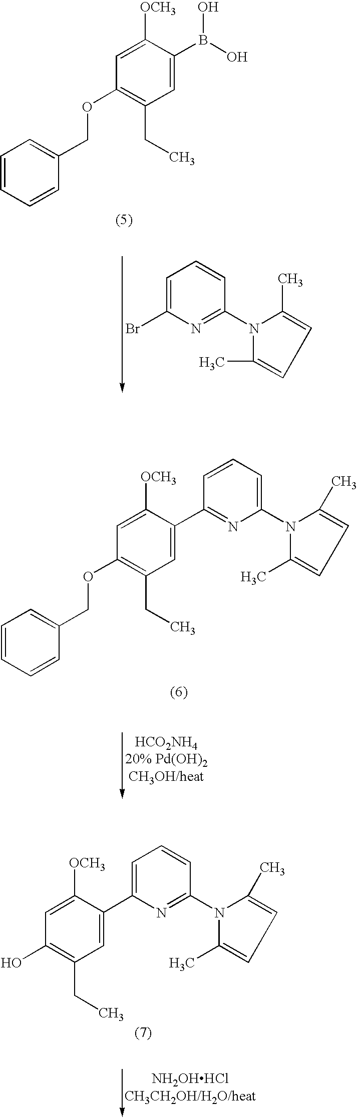

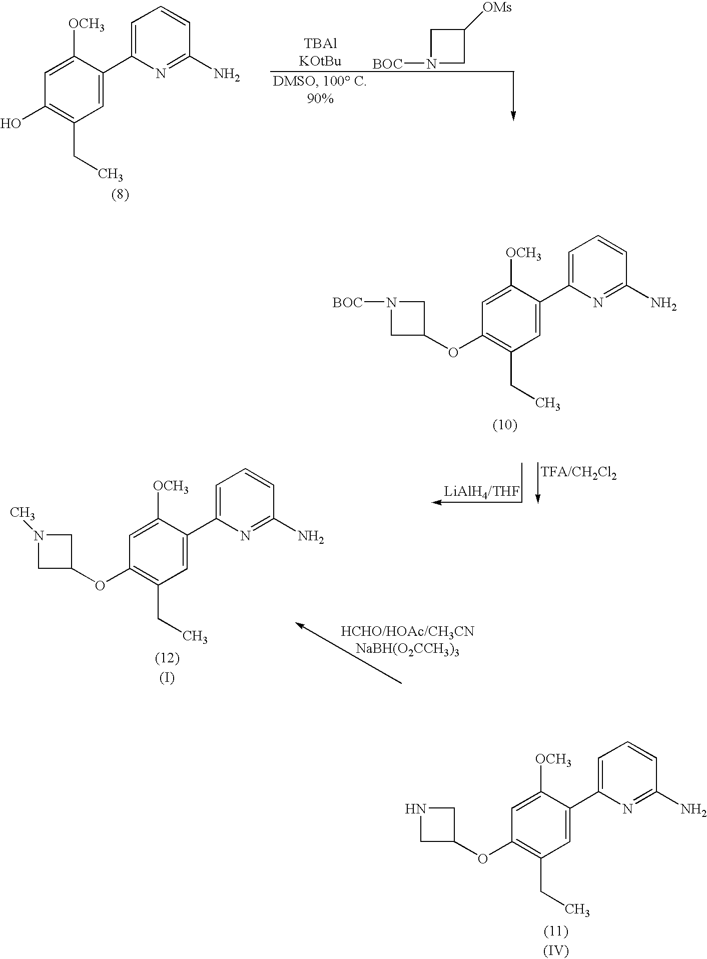

Scheme 1 illustrates a method for preparing the compound 6-[4-(N-methyl-3-azetidinoxy)-5-ethyl-2-methoxy-phenyl]-pyridin-2-ylamine, the compound of the formula I, and 6-[4-(3-azetidinoxy)-5-ethyl-2-methoxy-phenyl]-pyridin-2-ylamine, the compound of the formula II. These compounds are referred to in Scheme 1 as compounds of the formula “(I)” (or “(12)”) and “(IV)” (or (“(11)”), respectively.

The following reactions, which are illustrated in Scheme 1, are preferably conducted under a nitrogen atmosphere (unless otherwise indicated).

Referring to Scheme 1, 2-acetyl-5-methoxyphenol (1) may be reduced to 2-ethyl-5-methoxyphenol (2) by the methods described in Chem. Pharm. Bull. (Japan), 27 (1979) 1490-94. For example, 2-acetyl-5-methoxyphenol (1) can be treated with a reducing agent such as sodium borohydride in tetrahydrofuran (THF) along with a base such as triethylamine and an acylating agent such as ethyl chloroformate. Other tertiary amines and chloroformates can be used. While THF is the preferred solvent, diethyl ether can also be used. This reaction can be carried out at a temperature from about 0° C. to about 10° C., preferably about 0° C.

The alcohol group in the 2-ethyl-5-methoxyphenol (2) is protected by conversion to 3-benzyloxy-4-ethyl-1-methoxybenzene (3). More specifically 2-ethyl-5-methoxyphenol (2) is allowed to react with benzyl bromide and potassium carbonate in a polar solvent such as acetonitrile, dimethylformamide (DMF) or acetone, preferably acetone. The reaction yields 3-benzyloxy-4-ethyl-1-methoxybenzene (3). This reaction can be carried out at a temperature from about room temperature to about 60° C., preferably about 60° C.

Alternatively, 2-ethyl-5-methoxyphenol (2) may be allowed to react with benzyl bromide and potassium hydroxide in a polar solvent such as acetonitrile, dimethylsulfoxide (DMSO) or dimethylformamide (DMF), preferably acetonitrile. In this alternative reaction, a catalyst such as dibenzo-18-crown-6 may be used. This reaction also yields 3-benzyloxy-4-ethyl-1-methoxybenzene (3). The reaction is generally carried out at a temperature from about room temperature to about the reflux temperature of the reaction mixture, preferably at about the reflux temperature of the reaction mixture.

In a bromination reaction, the 3-benzyloxy-4-ethyl-1-methoxybenzene (3) is combined with N-bromosuccinimide (NBS) and silica gel 60 (EM Science, 480 Democrat Road, Gibbstown, N.J. 08027, an affiliate of Merck KGaA, Darmstadt, Germany) in a nonpolar solvent such as carbon tetrachloride at a temperature from about 0° C. to about room temperature. Preferably, the reaction is conducted at about room temperature. This reaction is allowed to stir, in the absence of light, to yield 5-benzyloxy-2-bromo-4-ethyl-1-methoxybenzene (4).

The resulting 5-benzyloxy-2-bromo-4-ethyl-1-methoxybenzene (4) is allowed to react with n-butyl lithium in a polar solvent such as ether, glyme or tetrahydrofuran (THF), preferably THF, at a temperature of about −78° C. Triethyl borate is then added to the reaction mixture, and the reaction mixture is allowed to stir at a temperature of about −78° C. The reaction mixture is then allowed to warm to about room temperature. The reaction yields 4-benzyloxy-5-ethyl-2-methoxy-phenylboronic acid (5).

Reacting the 4-benzyloxy-5-ethyl-2-methoxy-phenylboronic acid (5) with 2-bromo-6-(2,5-dimethylpyrrol-1-yl)pyridine, sodium carbonate and tetrakis(triphenylphosphine)palladium(0) in a polar solvent such as methanol/water, ethanol/water, or tetrahydrofuran (THF)/water, preferably ethanol/water, at a temperature from about room temperature to about the reflux temperature of the reaction mixture, preferably at about the reflux temperature, yields 2-(4-benzyloxy-5-ethyl-2-methoxy-phenyl)-6-(2,5-dimethyl-pyrrol-1-yl)-pyridine (6).

Alternatively, the 2-bromo-6-(2,5-dimethylpyrrol-1-yl)pyridine in the above reaction may be replaced with a compound having the structural formula

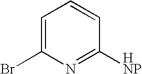

wherein P is a nitrogen protecting group such as trimethylacetyl or another appropriate nitrogen protecting group. Such protecting groups are well known to those of skill in the art. For example, nitrogen protecting groups are discussed in Greene, Theodora W. and Wuts, Peter G. M., Protective Groups In Organic Synthesis, Second Edition, John Wiley & Sons, Inc., New York, 1991 at pages 309-405. The above compounds are either commercially available, known in the scientific literature or easily obtained using well known methods and reagents.

The benzyl protecting group can be removed from the 2-(4-benzyloxy-5-ethyl-2-methoxy-phenyl)-6-(2,5-dimethyl-pyrrol-1-yl)-pyridine (6) by reacting this compound with ammonium formate in a polar solvent such as water or a lower alcohol solvent (e.g., methanol or ethanol), or in a mixture of one or more of these solvents, preferably methanol, at a temperature from about room temperature to about the reflux temperature of the reaction mixture. This reaction is preferably carried out at about the reflux temperature in the presence of about 20% palladium hydroxide on carbon. The aminopyridine protecting group is then removed from the resulting 4-[6-(2,5-dimethyl-pyrrol-1-yl)-pyridin-2-yl]-6-ethyl-3-methoxyphenol (7) in a conversion to 4-(6-amino-pyridin-2-yl)-2-ethyl-5-methoxyphenol (8). The 4-[6-(2,5-dimethyl-pyrrol-1-yl)-pyridin-2-yl]-6-ethyl-3-methoxyphenol (7) is converted to 4-(6-amino-pyridin-2-yl)-2-ethyl-5-methoxyphenol (8) by reacting it with hydroxylamine in a polar solvent such as water, a lower alcohol, such as methanol or ethanol, or a mixture of these solvents, preferably methanol/water. This reaction is conducted at a temperature from about room temperature to about the reflux temperature of the solvent, preferably at about the reflux temperature.

The 4-(6-amino-pyridin-2-yl)-2-ethyl-5-methoxyphenol (8) is treated with potassium t-butoxide and allowed to react with 3-methanesulfonyloxy-azetidine-1-carboxylic acid tert-butyl ester in a polar solvent such as dimethylsulfoxide (DMSO), dimethylformamide (DMF) or 1-methyl-2-pyrrolidinone, preferably DMSO, to form 6-[4-(3-azetidinoxy-1-carboxylic acid tert-butyl ester)-5-ethyl-2-methoxy-phenyl]-pyridin-2-ylamine (10). Other nitrogen protecting groups such as —C(═O)OCH₂C₆H₅, trifluoroacetyl and COOR (wherein R is benzyl, phenyl, alkyl, formyl or a similar group) can be used to protect the azetidine nitrogen. In addition, the mesylate leaving group can be replaced with another appropriate leaving group such as tosylate, trifluoroacetate or triflate. Other bases such as lithium t-butoxide can also be used. Preferably, a catalytic amount of tetrabutylammonium iodide (TBAI) is added to the reaction mixture. Other catalysts such as tetrabenzylammonium iodide and benzyltrimethylammonium iodide may also be used. This alkylation reaction is typically carried out in the presence of an alkali metal alkoxide such as lithium or potassium tert-butoxide, preferably potassium tert-butoxide, in a high boiling polar organic solvent such as DMSO, DMF or 1-methyl-2-pyrrolidinone, preferably DMSO. The reaction temperature can range from about 50° C. to about 100° C., and is preferably about 100° C.

Alternatively, 4-(6-amino-pyridin-2-yl)-2-ethyl-5-methoxyphenol (8) may be reacted with a compound having the structural formula

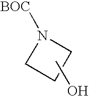

using triphenylphosphine and diethylazodicarboxylate or a water soluble azodicarboxylate in tetrahydrofuran (THF) under standard Mitsunobo reaction conditions to yield 6-[4-(3-azetidinoxy-1-carboxylic acid tert-butyl ester)-5-ethyl-2-methoxy-phenyl]-pyridin-2-ylamine (10). Typically, the reactants are combined at about 0° C. and then allowed to warm to room temperature.

Reduction of the 6-[4-(3-azetidinoxy-1-carboxylic acid tert-butyl ester)-5-ethyl-2-methoxy-phenyl]-pyridin-2-ylamine (10) yields the compound of formula I, 6-[4-(N-methyl-3-azetidinoxy)-5-ethyl-2-methoxy-phenyl]-pyridin-2-ylamine (12). This reduction is preferably accomplished using lithium aluminum hydride as the reducing agent and tetrahydrofuran (THF) or another organic ether (e.g., ethyl ether or glyme) as the solvent. Other aluminum hydride reducing agents can also be used, such as diisobutyl aluminum hydride. Diborane can also be used as the reducing agent. The foregoing reaction is generally conducted at a temperature from about room temperature to about the reflux temperature of the reaction mixture, preferably at about the reflux temperature.

Alternatively, the 6-[4-(3-azetidinoxy-1-carboxylic acid tert-butyl ester)-5-ethyl-2-methoxy-phenyl]-pyridin-2-ylamine (10) may be deprotected to yield the compound of formula IV, (6-[4-(3-azetidinoxy)-5-ethyl-2-methoxy-phenyl]-pyridin-2-ylamine (11). This transformation is preferably accomplished using trifluoroacetic acid (TFA) as the acid catalyst, either neat or in a polar solvent such as dichloromethane, chloroform or dichloroethane, preferably dichloromethane. Other acid catalysts can also be used, such as hydrochloric acid, hydrobromic acid or toluenesulfonic acid. This reaction is generally conducted at a temperature from about 0° C. to about room temperature, preferably at about room temperature.

The compound of formula IV, 6-[4-(3-azetidinoxy)-5-ethyl-2-methoxy-phenyl]-pyridin-2-ylamine (11), may itself be converted to the compound of formula I, 6-[4-(N-methyl-3-azetidinoxy)-5-ethyl-2-methoxy-phenyl]-pyridin-2-ylamine (12), by reductive amination. This reductive amination is preferably accomplished using formaldehyde, acetic acid and sodium triacetoxy borohydride as the reducing agent and acetonitrile/water, dichloromethane or methanol, preferably acetonitrile/water, as the solvent. Other reducing agents can also be used, such as sodium cyanoborohydride. The above reaction is generally conducted at a temperature from about 0° C., to about room temperature, preferably at about room temperature.

The starting materials used in the procedures of Scheme 1, the syntheses of which are not described above, are either commercially available, known in the art or readily obtainable from known compounds using methods that will be apparent to those skilled in the art.

The compounds of formulas I and IV, and the intermediates shown in the above reaction schemes, can be isolated and purified by conventional procedures, such as recrystallization or chromatographic separation.

Compounds of the formulas II and III, and their pharmaceutically acceptable salts, may be prepared as described in the following reaction schemes and discussion.

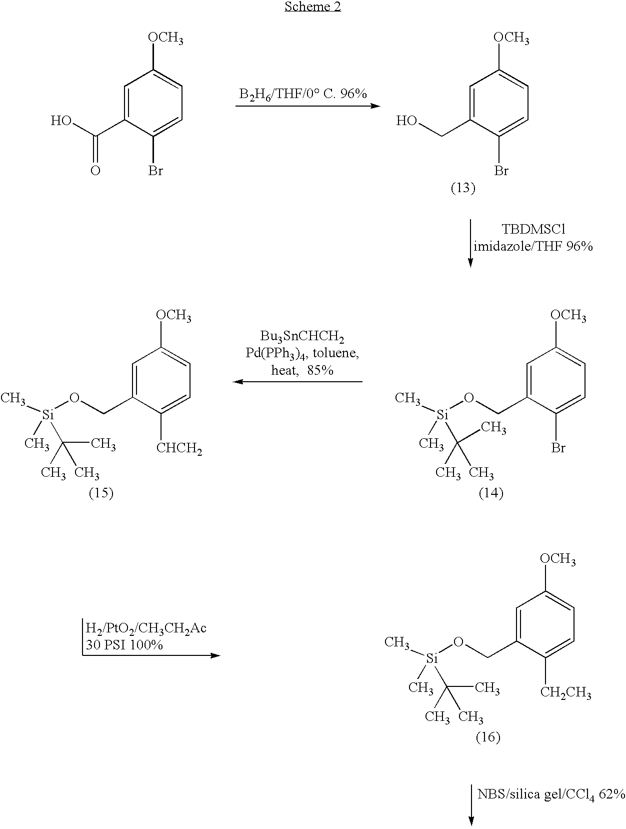

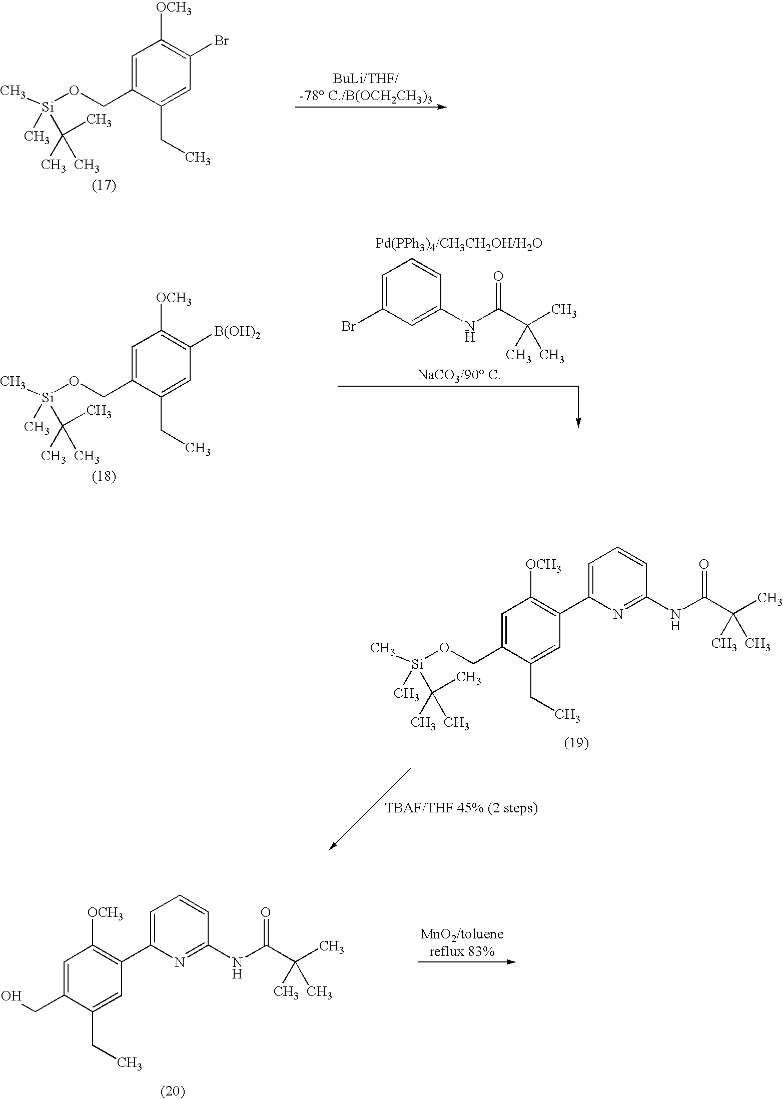

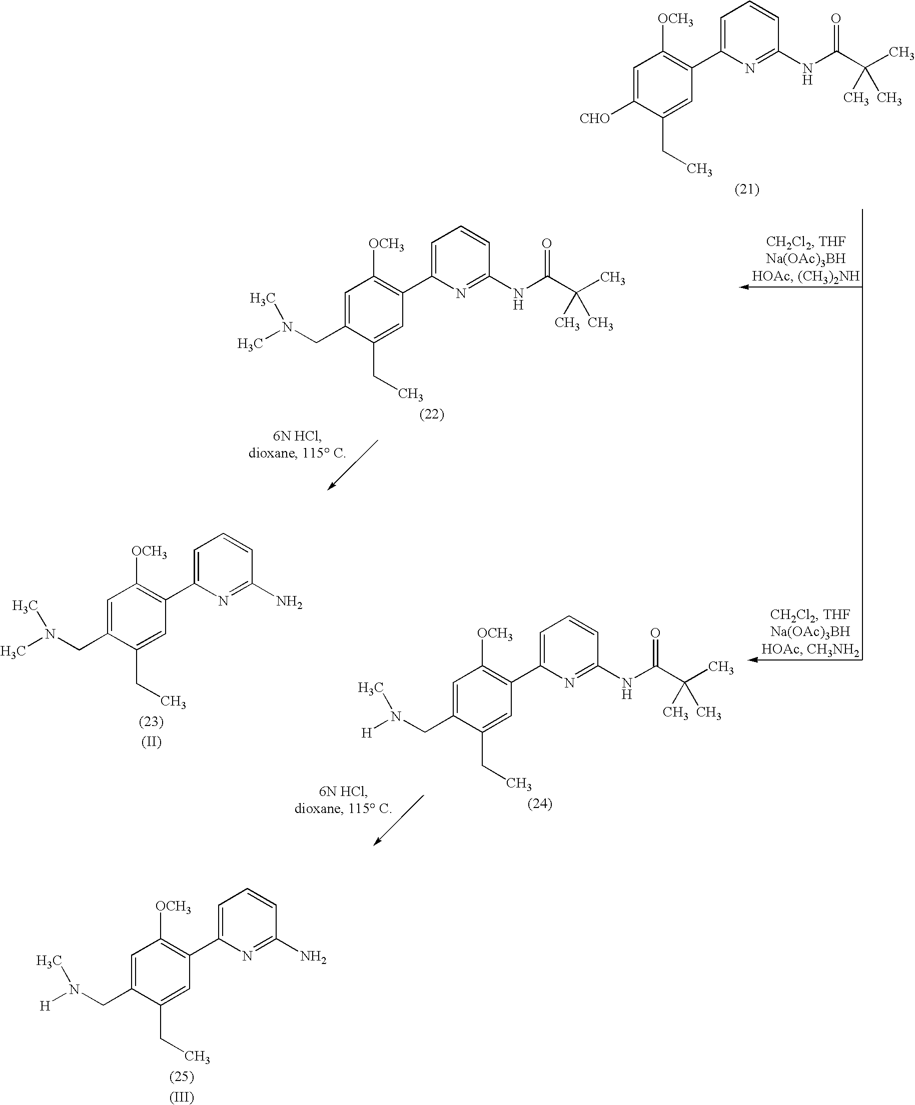

Scheme 2 illustrates a method for preparing the compound 6-[4-(N,N-dimethylaminomethyl)-5-ethyl-2-methoxy-phenyl]-pyridin-2-ylamine, the compound of the formula II, and 6-[4-(N-methylaminomethyl)-5-ethyl-2-methoxy-phenyl]-pyridin-2-ylamine, the compound of the formula III. These compounds are referred to in Scheme 2 as compounds of the formulas “(II)” (or “(23)”) and “(III)” (or “(25)”), respectively.

The following reactions, which are illustrated in Scheme 2, are preferably conducted under a nitrogen atmosphere (unless otherwise indicated).

Referring to Scheme 2, 2-bromo-5-methoxybenzoic acid is reduced to 2-bromo-5-methoxybenzyl alcohol (13) using borane (1 M in tetrahydrofuran (THF)) in a solvent such as THF, diethyl ether or diglyme, preferably THF. Other suitable reducing agents that may be used in the above transformation include BH₃.SMe₂ and lithium aluminum hydride/aluminum chloride. The reduction may be carried out at a temperature from about 0° C. to about room temperature, preferably about 0° C.

The alcohol group in the 2-bromo-5-methoxybenzyl alcohol (13) is protected by conversion to 2-bromo-5-methoxy-benzyloxy)-tert-butyl-dimethyl-silane (14). More specifically, 2-bromo-5-methoxybenzyl alcohol (13) is converted to 2-bromo-5-methoxy-benzyloxy)-tert-butyl-dimethyl-silane (14) with imidazole and t-butyl dimethylsilylchloride (TBDMSCI), or TBDMSOSO₂CF₃, in a solvent such as tetrahydrofuran (THF), dimethylformamide (DMF) or methylene chloride, preferably anhydrous THF, at a temperature from about 0° C. to about room temperature, preferably about room temperature.

In a Stille coupling reaction, 2-bromo-5-methoxy-benzyloxy)-tert-butyl-dimethyl-silane (14) is converted to tert-butyl-dimethyl-(2-vinyl-5-methoxy-benzyloxy)-silane (15). The conversion is carried out using tributylvinyl tin in a solvent such as toluene, dimethylformamide (DMF), acetone, xylene or benzene, preferably toluene, at a temperature from about room temperature to about 100° C., preferably about 100° C. A palladium catalyst such as tetrakis(triphenylphosphine)palladium(0) (Pd(PPh₃)₄), BnPdCl(PPh₃)₂, or PdCl₂(PPh₃)₂, preferably Pd(PPh₃)₄, may be used.

Tert-butyl-dimethyl-(2-vinyl-5-methoxy-benzyloxy)-silane (15) is reduced to tert-butyl-dimethyl-(2-ethyl-5-methoxy-benzyloxy)-silane (16) using a hydrogenation catalyst, preferably platinum oxide, under a hydrogen pressure of about 1 to 4 atmospheres, preferably under a hydrogen pressure of about 2 atmospheres. Suitable solvents include methanol, ethanol, ethyl acetate and acetic acid, preferably ethyl acetate. Catalysts such as 10% palladium (Pd) on calcium carbonate, Rh—C or Pd—C may also be used. The reaction is generally carried out at about room temperature.

In a bromination reaction, tert-butyl-dimethyl-(2-ethyl-5-methoxy-benzyloxy)-silane (16) is converted to tert-butyl-dimethyl-(4-bromo-2-ethyl-5-methoxy-benzyloxy)-silane (17) using N-bromosuccinimide (NBS) followed by the addition of silica gel 60 (EM Science, 480 Democrat Road, Gibbstown, N.J. 08027, an affiliate of Merck KGaA, Darmstadt, Germany). The reaction is allowed to stir in the absence of light. The reaction may also be carried out using NBS without silica gel, or using bromine instead of NBS. Suitable solvents include carbon tetrachloride chloroform, acetic acid and carbon disulfide, preferably carbon tetrachloride. The reaction may be carried out at about room temperature.

Tert-butyl-dimethyl-(4-bromo-2-ethyl-5-methoxy-benzyloxy)-silane (17) is cooled to about −78° C. in tetrahydrofuran (THF) and treated with n-butyl lithium. The reaction mixture is then treated with triethyl borate, at about −78° C., and allowed to warm to room temperature. Following acid workup, the reaction mixture yields tert-butyl-dimethyl-(4-boronic acid-2-ethyl-5-methoxy-benzyloxy)-silane (18). THF is the preferred solvent, but other suitable solvents such as diethyl ether may also be used. Similarly, n-butyl lithium is the preferred reagent, but other suitable reagents such as t-butyl lithium may also be used.

In a Suzuki coupling reaction, 2-bromo-6-(N-2,2-dimethylpropamido)pyridine and tert-butyl-dimethyl-(4-boronic acid-2-ethyl-5-methoxy-benzyloxy)-silane (18) are treated with sodium carbonate and tetrakis(triphenylphosphine)palladium(0) (Pd(PPh₃)₄) in ethanol and water. The reaction is heated to reflux to yield 2-2-(4-tert-butyldimethylsilyloxymethyl-5-ethyl-2-methoxy-phenyl)-6-(N-2,2-dimethylpropamido)-pyridine (19). Tetrakis(triphenylphosphine)palladium(0) is the preferred catalyst. However, other suitable palladium catalysts include Pd(OAc)₂, Pd₂(dba)₃ and [(allyl)PdCl]₂. Similarly, ethanol/water is the preferred solvent, but other suitable solvents such as tetrahydrofuran (THF), acetone, benzene and dimethoxyethane (DME) may be used.

The tert-butyl-dimethylsilyl protecting group is removed from the 2-2-(4-tert-butyldimethylsilyloxymethyl-5-ethyl-2-methoxy-phenyl)-6-(N-2,2-dimethylpropamido)-pyridine (19) by treatment with 1M tetrabutylammonium fluoride (TBAF) in tetrahydrofuran (THF) at about room temperature. The reaction yields 2-(4-hydroxymethyl-5-ethyl-2-methoxy-phenyl)-6-(N-2,2-dimethylpropamido)-pyridine (20). Although TBAF is the preferred reagent, other reagents such as KF/18-crown-6 and TBACI/KF may also be used. Similarly, although THF is the preferred solvent, other solvents such as diethyl ether and acetonitrile may be used.

The alcohol, 2-(4-hydroxymethyl-5-ethyl-2-methoxy-phenyl)-6-(N-2,2-dimethylpropamido)-pyridine (20), is oxidized to the corresponding aldehyde, 2-(4-formyl-5-ethyl-2-methoxy-phenyl)-6-(N-2,2-dimethylpropamido)-pyridine (21), by treatment with manganese dioxide in toluene. In addition to the preferred catalyst, manganese dioxide, other suitable catalysts include BaMnO₄ and AgMnO₄. Benzene may also be used as the solvent in the above reaction, although toluene is preferred. The above reaction is carried out at a temperature from about room temperature to about 100° C., preferably about 90° C.

Reductive amination of the aldehyde, 2-(4-formyl-5-ethyl-2-methoxy-phenyl)-6-(N-2,2-dimethylpropamido)-pyridine (21), with N,N-dimethylamine yields the amine, 2-(4-N,N-dimethylaminomethyl-5-ethyl-2-methoxy-phenyl)-6-(N-2,2-dimethylpropamido)-pyridine (22). This reductive amination is accomplished by treating 2-(4-formyl-5-ethyl-2-methoxy-phenyl)-6-(N-2,2-dimethylpropamido)-pyridine (21) in dichloromethane with N,N-dimethylamine in tetrahydrofuran (THF), sodium triacetoxyborohydride and acetic acid at about room temperature. Other suitable reducing agents include sodium cyanoborohydride.

The aminopyridine protecting group is removed from the 2-(4-N,N-dimethylaminomethyl-5-ethyl-2-methoxy-phenyl)-6-(N-2,2-dimethylpropamido)-pyridine (22) by treatment with 6N hydrogen chloride in dioxane at a temperature from about room temperature to about the reflux temperature, preferably at about the reflux temperature. The reaction yields the compound of formula II, 6-[4-(N,N-dimethylaminomethyl)-5-ethyl-2-methoxy-phenyl]-pyridin-2-ylamine (23). Aside from the 6N hydrogen chloride, which is preferred, other reagents that may be used in the above reaction include sodium hydroxide/methanol and barium hydroxide/methanol. Aside from dioxane, which is also preferred, other solvents that may be used include methanol/water and ethanol/water.

Alternatively, reductive amination of the aldehyde, 2-(4-formyl-5-ethyl-2-methoxy-phenyl)-6-(N-2,2-dimethylpropamido)-pyridine (21), with N-methylamine yields the amine, 2-(4-N-methylaminomethyl-5-ethyl-2-methoxy-phenyl)-6-(N-2,2-dimethylpropamido)-pyridine (24). This reductive amination is accomplished by combining 2-(4-formyl-5-ethyl-2-methoxy-phenyl)-6-(N-2,2-dimethylpropamido)-pyridine (21) in dichloromethane with N-methylamine in tetrahydrofuran (THF), acetic acid and sodium triacetoxyborohydride. The reaction is carried out at a temperature from about 0° C. to about room temperature, preferably about room temperature. Other suitable reducing agents include sodium cyanoborohydride.

The aminopyridine protecting group is removed from the 2-(4-N-methylaminomethyl-5-ethyl-2-methoxy-phenyl)-6-(N-2,2-dimethylpropamido)-pyridine (24) by treatment with dioxane and 6N hydrogen chloride at a temperature of about room temperature to about the reflux temperature, preferably at about the reflux temperature. The reaction yields the compound of formula III, 6-[4-(N-methylaminomethyl)-5-ethyl-2-methoxy-phenyl]-pyridin-2-ylamine (25). Aside from the 6N hydrogen chloride, which is preferred, other reagents which may be used in the above reaction include sodium hydroxide/methanol and barium hydroxide/methanol. Aside from dioxane, which is also preferred, other solvents which may be used include methanol/water and ethanol/water.

The starting materials used in the procedures of Scheme 2, the syntheses of which are not described above, are either commercially available, known in the art or readily obtainable from known compounds using methods that will be apparent to those skilled in the art.

The compounds of formulas II and III, and the intermediates shown in the above reaction schemes can be isolated and purified by conventional procedures, such as recrystallization or chromatographic separation.

Compounds of formula VI as defined above can be synthesized by generally following the Schemes provided above. Compounds of formula VI can also be synthesized according to the following Schemes 3-5. Schemes 6-9 provide more specific examples of synthesis of compounds of formula VI. The bold numbers in the Schemes refer to the compounds in the Examples, below, having the same bold number. Compounds of formula VI can be synthesized by generally following the procedures illustrated in the following Schemes.

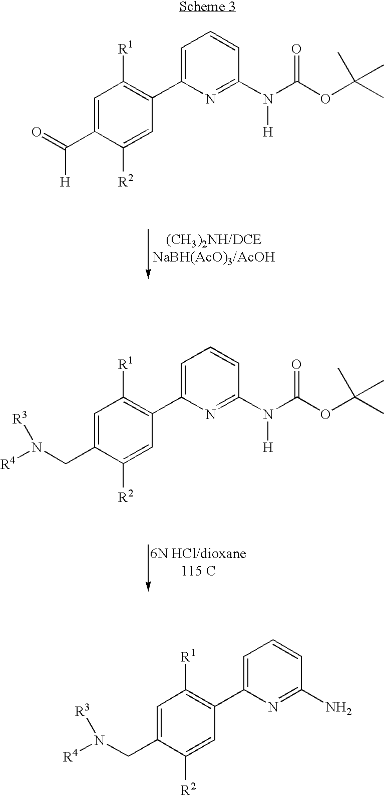

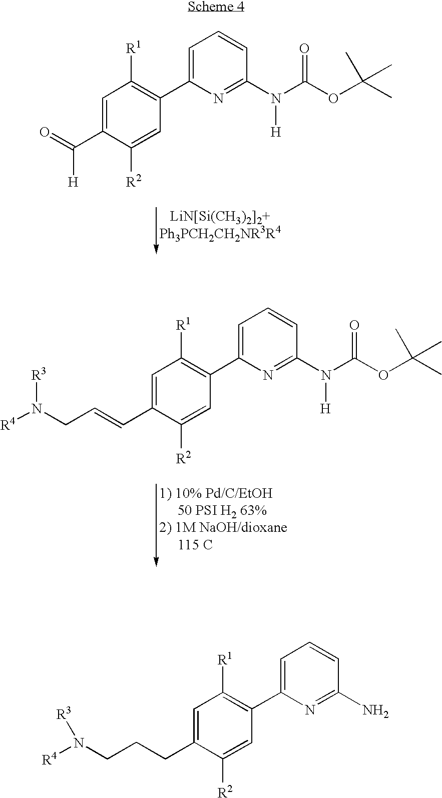

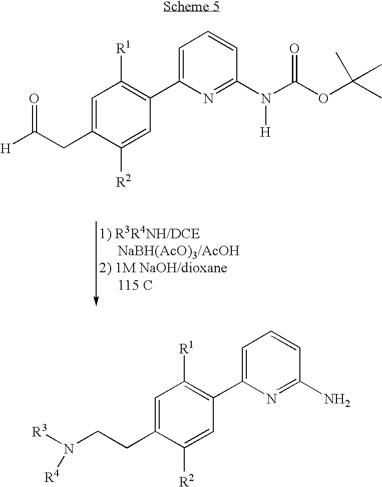

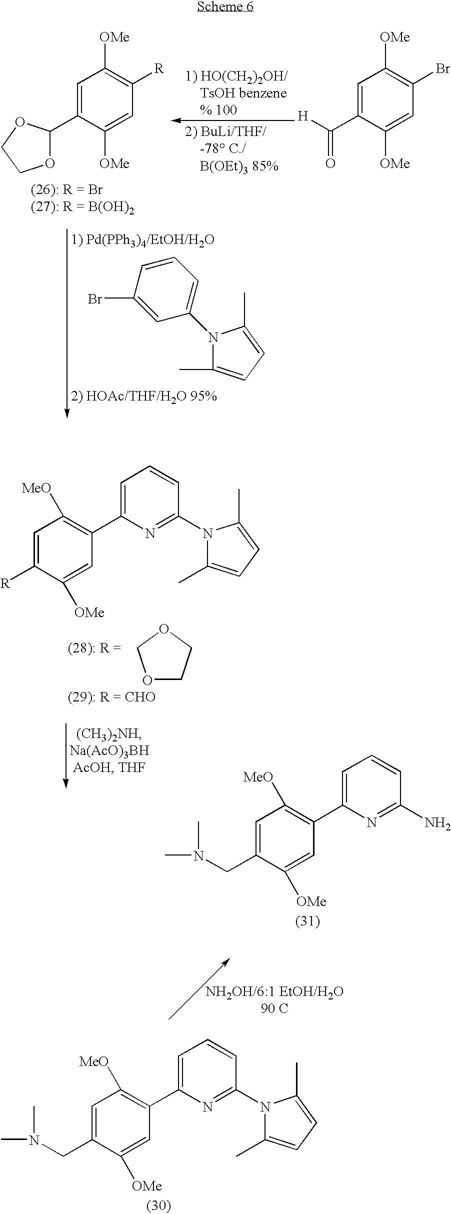

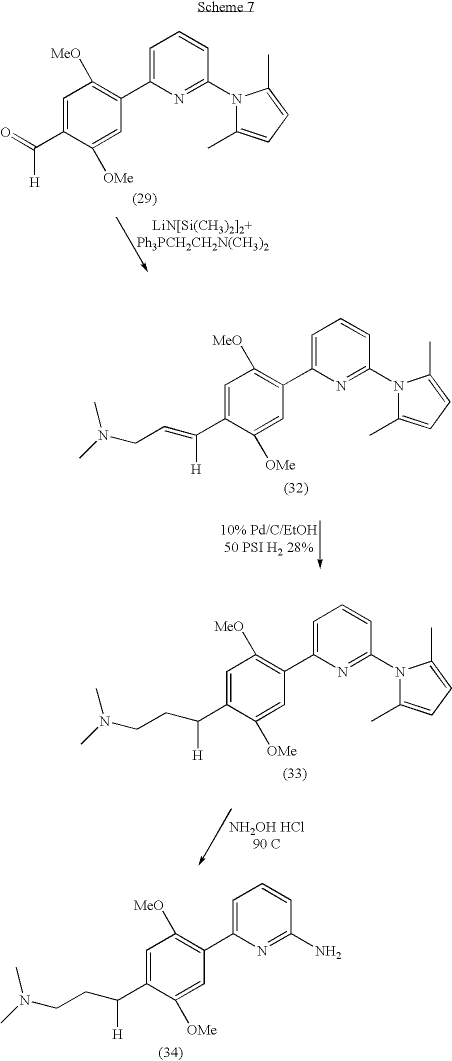

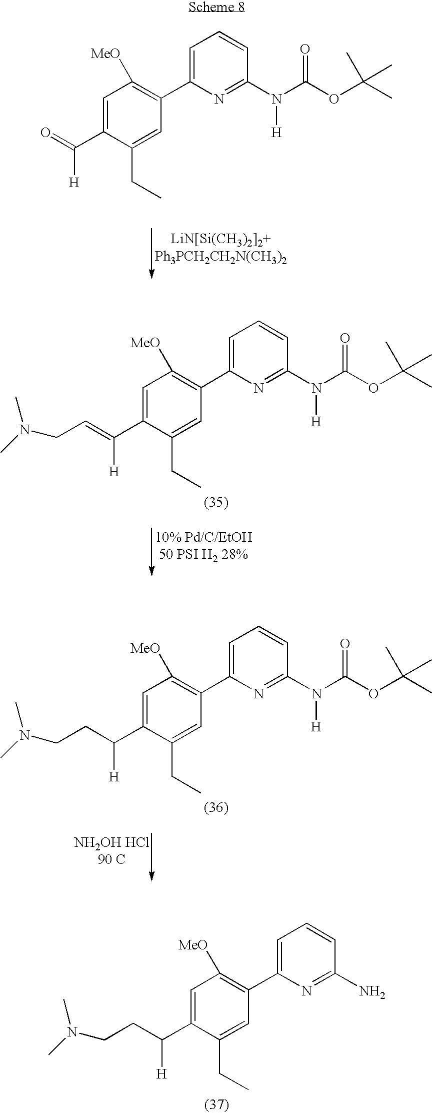

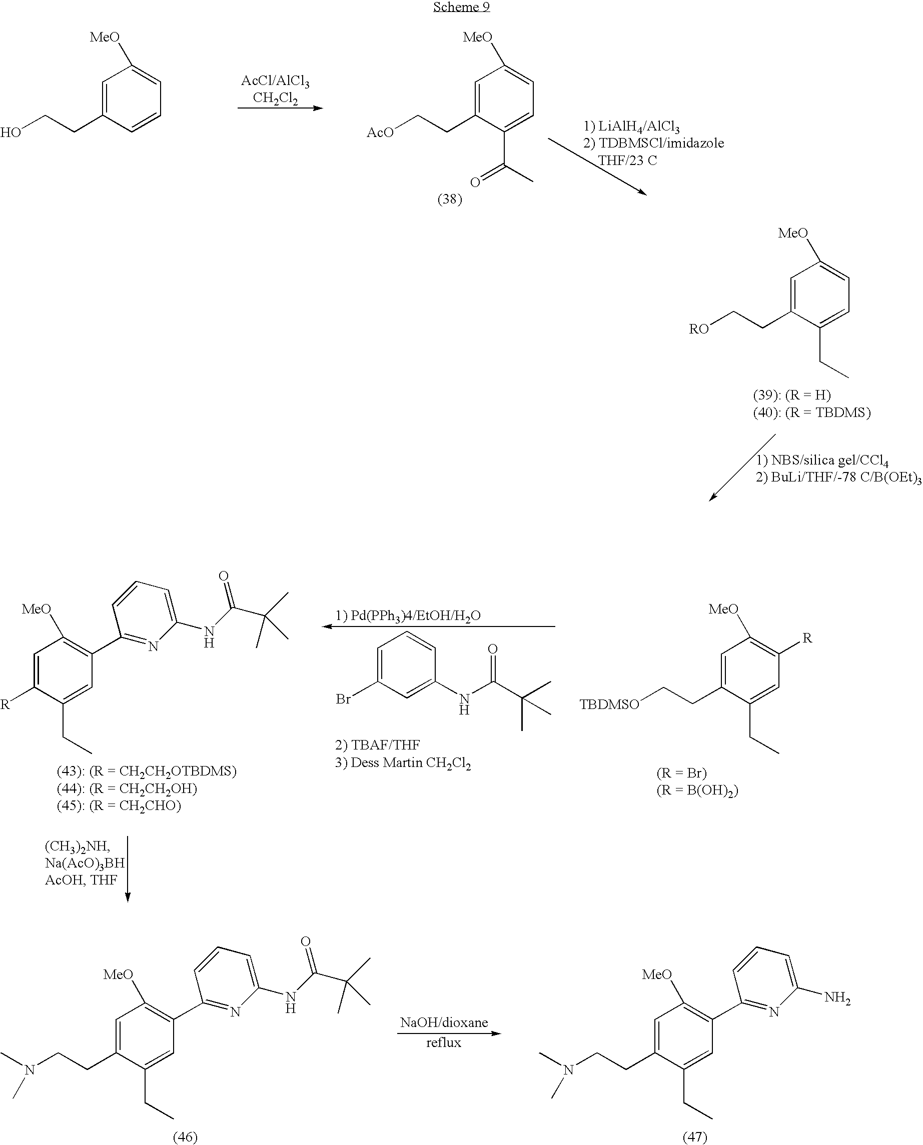

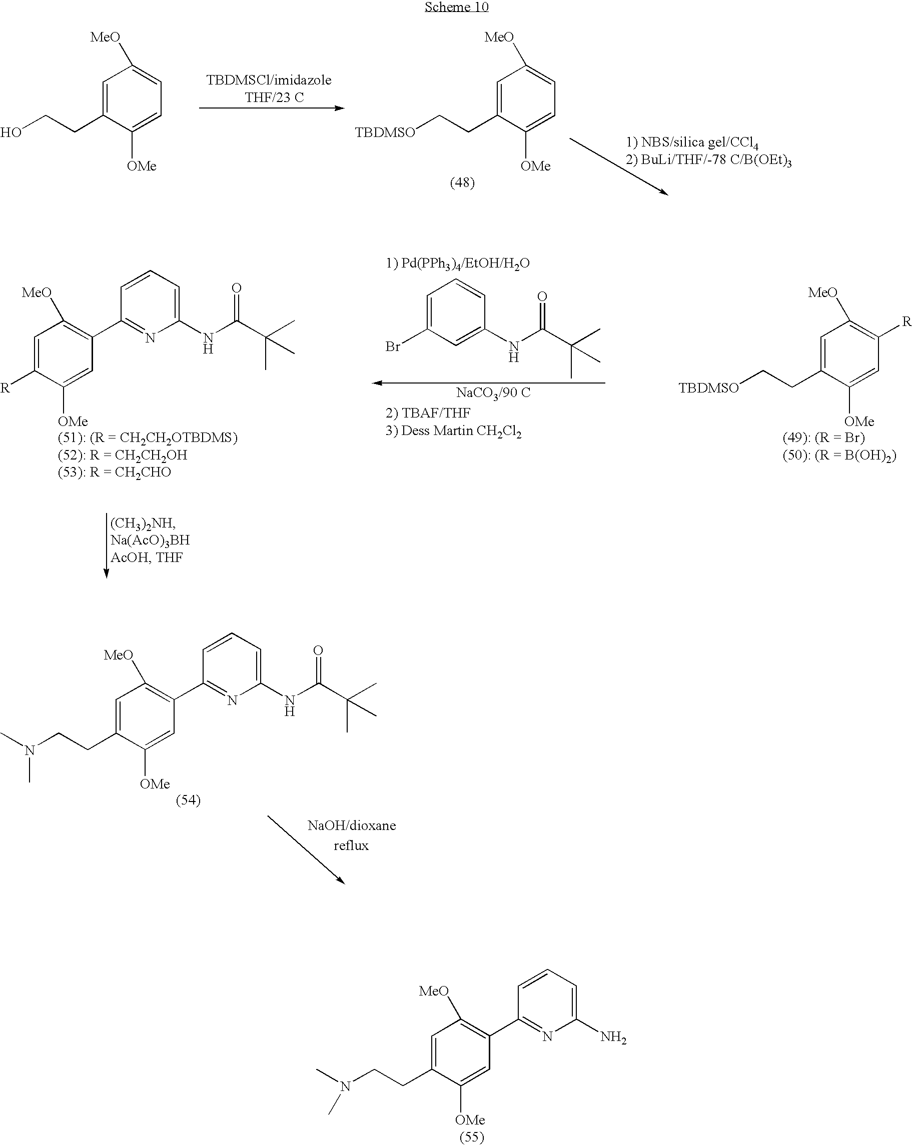

Referring to Scheme 10, above, compounds analogous to (55), but wherein the dimethylamino-ethyl group is replaced with a different substituted amine group of the formula R³R⁴N-ethyl, can be synthesized under the same reaction conditions using any primary or secondary amine R³R⁴NH instead of (CH₃)₂NH during the conversion from (53) to (54).

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

This invention relates to the compounds of formulas I, II, III, IV, and VI, and their pharmaceutically acceptable salts. The compounds of formulas I, II, III, IV, and VI, and their pharmaceutically acceptable salts, are hereinafter collectively referred to as “the active compounds of the present invention.” The active compounds of the present invention can be administered to mammals via either the oral, parenteral (such as subcutaneous, intravenous, intramuscular, intrasternal and infusion techniques), rectal, intranasal or topical routes. In general, these compounds are most desirably administered in doses ranging from about 0.01 mg to about 1500 mg per day, in single or divided doses (i.e., from 1 to 4 doses per day), although variations will necessarily occur depending upon the species, weight and condition of the subject being treated and the particular route of administration chosen. However, a dosage level that is in the range of about 0.5 mg to about 500 mg per kg of body weight per day is most desirably employed. Nevertheless, variations may occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effects, provided that such higher dosage levels are first divided into several small doses for administration throughout the day.

The active compounds of the present invention may be administered alone or in combination with pharmaceutically acceptable carriers or diluents by either of the routes previously indicated, and such administration may be carried out in single or multiple doses. More particularly, the active compounds of the present invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders such as polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules. Preferred materials in this connection include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions containing an active compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well known to those skilled in the art.

Additionally, it is also possible to administer the active compounds of the present invention topically when treating inflammatory conditions of the skin. This may be done by way of creams, jellies, gels, pastes, patches, ointments and the like, in accordance with standard pharmaceutical practice.

The active compounds of the present invention are useful as NOS inhibitors i.e., they possess the ability to inhibit the NOS enzyme in mammals, and therefore they are able to function as therapeutic agents in the treatment of the aforementioned disorders and diseases in an afflicted mammal.

The ability of compounds of the formulas I, II, III IV, and VI of this invention, and their pharmaceutically acceptable salts, to inhibit NOS may be determined using procedures described in the literature. The ability of compounds of the present invention to inhibit endothelial NOS may be determined by using the procedures described by Schmidt et al. in Proc. Natl. Acad. Sci. U.S.A., 88, pp. 365-369 (1991) and by Pollock et al., in Proc. Natl. Acad. Sci. U.S.A., 88, pp. 10480-10484 (1991). The ability of compounds of the present invention to inhibit inducible NOS may be determined using the procedures described by Schmidt et al., in Proc. Natl. Acad, Sci. U.S.A., 88 pp. 365-369 (1991) and by Garvey et al. in J. Biol. Chem., 269, pp. 26669-26676 (1994). The ability of the compounds of the present invention to inhibit neuronal NOS may be determined using the procedure described by Bredt and Snyder in Proc. Natl. Acad. Sci. U.S.A., 87, 682-685 (1990).

As indicated above, inhibition of NO synthase activity may be determined by conversion of [³H]arginine to [³H]citrulline as described by Bredt and Snyder in Proc. Natl. Acad. Sci. U.S.A. 87, 682-685 (1990), but with slight modification. Specifically, 10 uL of crude enzyme lysate and 10 uL of 350 nM [³H]arginine are added to 100 uL of buffer containing 10 mM Hepes, 0.32 M sucrose, 0.75 mM NADPH, 0.1 mM EDTA, 0.63 mM CaCl₂, 1 mM dithiothreitol, 30 nM calmodulin (CaM), 2 uM Flavin Adenin dinucleotie (FAD), 2 uM Flavin mononucleotide (FMN), 3 uM tetrahydrobiopterin (H₄B) and trace bovine serum albumin in 96-well plate format. After incubation for 50 minutes at 30° C., assays are terminated by application to 75 uL BioRex-70 resin (H⁺ form) and eluted with 90 uL of water. [³H]citrulline may be quantified by liquid scintillation spectroscopy of the total flow-through.

The title compounds of Examples 1-4 below were tested according to the foregoing procedure and each exhibited an IC₅₀<1 μM for inhibition of neuronal NOS.

The following Examples illustrate different embodiments of the present invention. It will be understood, however, that the invention is not limited to the specific details of these examples. Melting points are uncorrected. Proton nuclear magnetic resonance spectra (¹H NMR) and ¹³C nuclear magnetic resonance spectra were measured for solutions in deuterochloroform (CDCl₃) or in CD₃OD or CD₃SOCD₃ and peak positions are expressed in parts per million (ppm) downfield from tetramethylsilane (TMS). The peak shapes are denoted as follows: s, singlet; d, doublet; t, triplet; q, quartet, m, multiplet, b, broad.

### Preparation 1

### 2-ethyl-5-methoxyphenol (2)

Under a nitrogen atmosphere, 36.70 g (120.4 mmol) of 2-acetyl-5-methoxyphenol (1) was combined with 20.13 ml (144.4 mmol) of triethylamine in 150 mL of anhydrous THF. The reaction mixture was cooled to 0° C., and 13.81 ml (144.4 mmol) of ethyl chloroformate was added dropwise to the reaction mixture over a 30 minute period. The reaction mixture was allowed to stir for an additional 30 minutes. The resultant white solids were filtered. A solution of 13.64 g (361.1 mmol) of sodium borohydride in 200 ml of water was added dropwise to the filtrate over a period of 45 minutes at a temperature of 5-10° C. The reaction mixture was allowed to warm to room temperature and stirred for 1.5 hours. The resultant solution was acidified to pH 2 with 1 M HCl and extracted with ether (1×250 ml). The ether layer was then extracted with 10% sodium hydroxide (5×100 mL). The combined base extracts were acidified with concentrated HCl and extracted with ether. The combined ether extracts were washed with water (1×100 ml), dil NaHCO₃ (1×100 ml) and brine (1×100 mL), dried over sodium sulfate, filtered and concentrated in vacuo to yield 18.37 g of crude product, 2-ethyl-5-methoxyphenol (2), as a colorless oil. The crude product was used in Preparation 2 below.

¹H NMR (CDCl₃): 1.20 (t-3H; J=7.26 Hz), 1.87 (bs-1H), 2.55 (q-2H), 3.75 (s-3H), 6.35 (d-1H, J=0.5 Hz), 6.44 (dd-1H), 7.01 (d-1H, J=8.3 Hz).

### Preparation 2

### 3-benzyloxy-4-ethyl-1-methoxybenzene (3)

Under a nitrogen atmosphere, 18.30 g (120.2 mmol) of 2-ethyl-5-methoxyphenol (2) was dissolved in 150 ml of acetone. To this solution was added 33.249 (240.5 mmol) of potassium carbonate followed by 15.02 ml (126.3 mmol) of benzyl bromide. The resultant mixture was heated to reflux for 16 hours. The reaction mixture was concentrated in vacuo and partitioned between ethyl acetate (300 ml) and water (300 ml). The ethyl acetate layer was separated, washed with 1 M NaOH (2×200 mL) and brine (1×200 mL), dried over sodium sulfate, filtered and concentrated in vacuo to yield 29.70 g of crude 3-benzyloxy-4-ethyl-1-methoxybenzene (3). Chromatography of the crude product on 400 g of silica gel 60 (EM Science) using 97:3 hexane:ethyl acetate yielded 12.62 g (43%) of 3-benzyloxy-4-ethyl-1-methoxybenzene (3).

¹H NMR (CDCl₃): 1.20 (t-3H; J=7.47 Hz), 2.64 (q-2H; J=7.47 Hz), 3.78 (s-3H), 5.06 (s-2H), 6.45 (dd-1H, J=2.29, 8.30 Hz), 6.50 (d-1H; J=2.28 Hz), 7.07 (d-1H, J=8.30 Hz), 7.30-7.45 (m-5H).

### Preparation 3

### 5-benzyloxy-2-bromo-4-ethyl-1-methoxybenzene (4)

Under a nitrogen atmosphere, 12.60 g (52.00 mmol) of 3-benzyloxy-4-ethyl-1-methoxybenzene (3) and 9.72 g (54.60 mmol) of NBS were combined in 350 mL of carbon tetrachloride, followed by the addition of 50 g of silica gel 60 (EM Science). The reaction was allowed to stir for 16 hours in the absence of light. The reaction mixture was filtered and the silica gel was washed with dichloromethane. The filtrate was washed with ethyl acetate (1×300 mL). The combined organic extracts were washed with 1M NaOH (2×300 mL), dilute NaHSO₃ (1×200 mL) and brine (1×200 mL), dried over magnesium sulfate, filtered and concentrated in vacuo to yield 16.82 g (100%) of crude, 5-benzyloxy-2-bromo-4-ethyl-1-methoxybenzene (4) as a colorless oil.

¹H NMR (CDCl₃): 1.17 (t-3H; J=7.48 Hz), 2.60 (q-2H; J=7.48 Hz), 3.82 (s, 3H), 5.07 (s-2H), 6.50 (s, 1H), 7.25-7.44 (m-6H).

### Preparation 4

### 4-benzyloxy-5-ethyl-2-methoxy-phenylboronic Acid (5)

Under a nitrogen atmosphere, 16.70 g (52.00 mmol) of 5-benzyloxy-2-bromo-4-ethyl-1-methoxybenzene (4) was added to 110 mL of anhydrous THF. The solution was cooled to −78° C., and 22.88 mL (57.19 mmol) of a 2.5 M solution of butyl lithium was added dropwise while maintaining the temperature below −70° C. The reaction mixture was stirred at −78° C. for 45 minutes. 9.73 mL (57.19 mmol) of triethyl borate was then added, and the reaction was allowed to stir at −78° C. for an additional 2 hours. The reaction mixture was then allowed to warm to 23° C. over a 30 minute period and was quenched with 100 mL of saturated NH₄Cl. The pH was adjusted to 5.0 with 1M HCl, and the resultant solution was extracted with ethyl acetate (2×200 mL). The combined extracts were washed with brine (1×100 mL), dried over sodium sulfate, filtered and concentrated in vacuo to yield crude product as a greenish-tan solid. The crude product was triturated with hexane and filtered to afford 10.65 g (64%) of 4-benzyloxy-5-ethyl-2-methoxy-phenylboronic acid (5) as an off-white solid.

¹H NMR (CDCl₃): 1.19 (t-3H), 2.62 (q-2H), 3.85 (s, 3H), 5.13 (s-2H), 5.77 (bs, 2H), 6.47 (s, 1H), 7.25-7.59 (m-6H).

### Preparation 5

### 2-(4-benzyloxy-5-ethyl-2-methoxy-phenyl)-6-(2,5-dimethyl-pyrrol-1-yl)-pyridine (6)

Under a nitrogen atmosphere, 5.00 g (19.91 mmol) of 2-bromo-6-(2,5-dimethylpyrrol-1-yl)pyridine, 5.98 g (20.91 mmol) of 4-benzyloxy-5-ethyl-2-methoxy-phenylboronic acid (5), 8.44 g (79.64 mmol) of sodium carbonate and 1.15 g (0.996 mmol) of tetrakis(triphenylphosphine)palladium(0) were combined in 90 mL of ethanol and 10 mL of water. The solution was allowed to reflux for 64 hours, and then the reaction mixture was concentrated in vacuo. The resultant yellow residue was partitioned between ethyl acetate (200 mL) and water (200 mL). The aqueous layer was extracted again with ethyl acetate (200 mL). The combined organic extracts were washed with brine (1×200 mL), dried over sodium sulfate, filtered and concentrated in vacuo to yield crude product as a yellow oil which crystallized upon standing. Recrystallization of this solid from absolute ethanol afforded 6.00 g (73%) of 2-(4-benzyloxy-5-ethyl-2-methoxy-phenyl)-6-(2,5-dimethyl-pyrrol-1-yl)-pyridine (6) as a tan solid.

¹H NMR (CDCl₃): 1.21 (t-3H; J=7.47 Hz), 2.22 (s-6H), 2.67 (q-2H; J=7.47 Hz), 3.85 (s, 3H), 5.15 (s-2H), 5.91 (s-2H), 6.56 (s, 1H), 7.04-7.91 (m-9H).

### Preparation 6

### 4-[6-(2,5-Dimethyl-pyrrol-1-yl)-pyridin-2-yl]-6-ethyl-3-methoxyphenol (7)

Under a nitrogen atmosphere, 5.90 g (14.30 mmol) of 2-(4-benzyloxy-5-ethyl-2-methoxy-phenyl)-6-(2,5-dimethyl-pyrrol-1-yl)-pyridine (6) and 27.06 g (429.1 mmol) of ammonium formate were combined in 125 mL of methanol and 500 mg of 20% Pd(OH)₂ on carbon. The resultant slurry was allowed to reflux for 45 minutes. 500 mg of 20% Pd(OH)₂ on carbon was added twice more, and the resultant slurry was allowed to reflux for 45 minutes. The reaction mixture was then allowed to cool to ambient temperature and passed through a pad of celite to remove the catalyst. The filtrate was concentrated in vacuo and the resultant yellow residue was partitioned between ethyl acetate (200 mL) and water (200 mL). The aqueous layer was extracted again with ethyl acetate (200 mL). The combined organic extracts were washed with brine (1×200 mL), dried over sodium sulfate, filtered and concentrated in vacuo to yield 4.52 g (98%) of 4-[6-(2,5-Dimethyl-pyrrol-1-yl)-pyridin-2-yl]-6-ethyl-3-methoxyphenol (7) as a tan solid.

¹H NMR (CDCl₃): 1.20 (t-3H; J=7.41 Hz), 2.20 (s-6H), 2.55 (q-2H; J=7.41 Hz), 3.82 (s, 3H), 5.45 (bs-1H), 5.90 (s-2H), 6.50 (s, 1H), 7.04 (dd-1H; J=0.99, 7.74 Hz), 7.70 (s-1H), 7.76-7.91 (m-2H).

### Preparation 7

### 4-(6-Amino-pyridin-2-yl)-2-ethyl-5-methoxyphenol (8)

Under a nitrogen atmosphere, 4.50 g (13.96 mmol) of 4-[6-(2,5-Dimethyl-pyrrol-1-yl)-pyridin-2-yl]-6-ethyl-3-methoxyphenol (7) and 11.64 g (167.5 mmol) of hydroxylamine hydrochloride were combined in 84 mL of ethanol and 14 mL of water. The resultant mixture was allowed to reflux for 16 hours. The reaction mixture was then allowed to cool to ambient temperature and concentrated in vacuo. The resultant yellow residue was partitioned between ethyl acetate (200 mL) and dilute sodium bicarbonate (200 mL). The aqueous layer was extracted again with ethyl acetate (2×200 mL). The combined organic extracts were washed with brine (1×200 mL), dried over sodium sulfate, filtered and concentrated in vacuo to yield crude product as a brown solid. Chromatography of the crude product on 250 g of silica gel 60 (EM Science) using 4:1 ethyl acetate:hexane yielded 1.86 g (55%) of 4-(6-Amino-pyridin-2-yl)-2-ethyl-5-methoxyphenol (8) as a salmon colored solid.

¹H NMR (CD₃OD): 1.21 (t-3H; J=7.41 Hz), 1.87 (s-2H), 2.40 (s-1H), 2.61 (q-2H; J=7.41 Hz), 3.78 (s-3H), 6.46 (dd-1H, J=0.82, 8.14 Hz), 6.52 (s-1H), 6.92 (dd-1H, J=0.82, 7.41), 7.26 (s-1H), 7.45 (dd-1H).

### Preparation 8

### 6-[4-(3-azetidinoxy-1-carboxylic acid tert-butyl ester)-5-ethyl-2-methoxy-phenyl]-pyridin-2-ylamine (10)

Under a nitrogen atmosphere, 100 mg (0.41 mmol) of 4-(6-Amino-pyridin-2-yl)-2-ethyl-5-methoxyphenol (8) and 92 mg (0.82 mmol) of potassium t-butoxide were combined in 8 mL of anhydrous DMSO. The reaction mixture was allowed to stir for 10 minutes. 206 mg (0.82 mmol) of 3-methanesulfonyloxy-azetidine-1-carboxylic acid tert-butyl ester in 2 mL of anhydrous DMSO was added to the reaction mixture, followed by the addition of 50 mg of tetrabutylammonium iodide. The reaction mixture was heated to 100° C. and stirred for 18.5 hours. The reaction mixture was then allowed to cool to room temperature. Ethyl acetate (100 mL) was added, and the solution was washed with 1N NaOH (1×100 mL) and brine (1×100 mL), dried over sodium sulfate, filtered and concentrated in vacuo to yield crude product. The crude product was chromatographed on a Flash 12M silica gel column with 100% ethyl acetate. The crude product was then concentrated in vacuo and rechromatographed with 0 to 3% methanol in dichloromethane to afford 142 mg (87%) of 6-[4-(3-azetidinoxy-1-carboxylic acid tert-butyl ester)-5-ethyl-2-methoxy-phenyl]-pyridin-2-ylamine (10) as an off-white solid.

¹H NMR (CDCl₃): 1.18 (t-3H; J=7.3 Hz), 1.44 (s-9H), 2.60 (s-2H), 2.61 (q-2H), 3.76 (s-3H), 4.00-4.44 (m-4H), 4.92 (m-1H), 6.10(s-1H), 6.39 (m-1H), 7.10 (m-1H), 7.42 (m-1H), 7.53 (s-1H).

### EXAMPLE 1

### 6-[4-(N-methyl-3-azetidinoxy)-5-ethyl-2-methoxy-phenyl]-pyridin-2-ylamine (12)

Under a nitrogen atmosphere, 357 mg (0.92 mmol) of crude 6-[4-(3-azetidinoxy-1-carboxylic acid tert-butyl ester)-5-ethyl-2-methoxy-phenyl]-pyridin-2-ylamine (10) and 39 mg (1.02 mmol) of lithium aluminum hydride were added to 15 mL of anhydrous THF. The reaction mixture was heated to reflux for 16 hours, and then an additional 75 mg (2.04 mmol) of lithium aluminum hydride was added. After 2 hours, the reaction mixture was allowed to cool to ambient temperature. The reaction mixture was then carefully quenched sequentially with 114 ul of water, 114 ul of 1 N NaOH and 342 ul of water. The aluminum salts were filtered and washed with ethyl acetate, and the filtrate was dried over sodium sulfate and concentrated in vacuo to yield crude product. The crude product was chromatographed on a Flash 12M silica gel column with 5 to 10% methanol in dichloromethane to afford 48 mg (38%) of 6-[4-(N-methyl-3-azetidinoxy)-5-ethyl-2-methoxy-phenyl]-pyridin-2-ylamine (12). The 6-[4-(N-methyl-3-azetidinoxy)-5-ethyl-2-methoxy-phenyl]-pyridin-2-ylamine (12) was converted to its dihydrochloride salt by dissolving the aminopyridine (12) in dichloromethane and adding 1 ml of an ether solution saturated with HCl. The resultant residue was concentrated and triturated with ethylacetate.

¹H NMR (CDCl₃): 1.17 (t-3H; J=7.58 Hz), 2.40 (s-3H), 2.59 (m-2H), 3.07-3.11 (m-2H), 3.75 (s-3H), 3.83-3.89 (m-2H), 4.39 (bs-2H), 4.77 (m-1H), 6.20 (s-1H), 6.37 (d-1H; J=8.07 Hz), 7.09 (m-1H), 7.41 (m-1H), 7.51 (s-1H).

### EXAMPLE 2

### 6-[4-(3-azetidinoxy)-5-ethyl-2-methoxy-phenyl]-pyridin-2-ylamine (11)

Under a nitrogen atmosphere, 82 mg (0.21 mmol) of 6-[4-(3-azetidinoxy-1-carboxylic acid tert-butyl ester)-5-ethyl-2-methoxy-phenyl]-pyridin-2-ylamine (10) and 10 mL of TFA were added to 20 mL of dichloromethane. The reaction mixture was allowed to stir for 1.5 hours at ambient temperature, and then concentrated in vacuo to yield crude amine. This crude product was partitioned between saturated NaHCO₃ (25 mL) and dichloromethane (100 mL). The organic layer was dried over sodium sulfate, filtered and concentrated in vacuo. This material was chromatographed on a Flash 12M silica gel column, beginning with 1% and increasing to 10% methanol in dichloromethane, to afford 26 mg (43%) of 6-[4-(3-azetidinoxy)-5-ethyl-2-methoxy-phenyl]-pyridin-2-ylamine (11).

¹H NMR (CDCl₃): 1.17 (t-3H), 2.59 (m-2H), 3.20 (bs-1H), 3.76 (s-3H), 3.80-4.00 (m-4H), 4.50 (bs-2H), 5.00 (m-1H), 6.14 (s-1H), 6.37 (m-1H), 7.07 (m-1H), 7.42 (m-1H), 7.50 (s-1H).

### Preparation 9

### 2-bromo-5-methoxybenzyl Alcohol (13)

Under a nitrogen atmosphere, 25 g (0.11 mol) of 2-bromo-5-methoxybenzoic acid was dissolved in 100 mL of anhydrous THF. 140 mL (0.14 mol) of borane (1 M in THF) was added to this solution over a period of 1 hour. The reaction was allowed to stir and was carefully quenched with 1:1 THF: saturated K₂CO₃. Ether was added and the aqueous and organic layers were separated. The aqueous layer was extrated again with ether (2×100 mL). The combined organic extracts were dried over sodium sulfate, filtered and concentrated in vacuo to afford 23.0 g (96%) of 2-bromo-5-methoxybenzyl alcohol (13).

¹H NMR (CDCl₃): 2.02 (t-1H; J=6.23 Hz), 3.79 (s-3H), 4.69 (d-2H; J=6.23 Hz), 6.70 (dd-1H; J=3.12, 8.72 Hz), 7.04 (d-1H; J=3.12 Hz), 7.39 (d-1H; J=8.72 Hz).

### Preparation 10

### (2-bromo-5-methoxy-benzyloxy)-tert-butyl-dimethyl-silane (14)

Under a nitrogen atmosphere, 23.0 g (0.11 mol) of 2-bromo-5-methoxybenzyl alcohol (13) was dissolved in 100 mL of anhydrous THF. 14.43 g (0.21 mol) of imidazole was added, followed by 17.6 g (0.12 mol) of t-butyl dimethylsilylchloride. The reaction mixture was allowed to stir overnight at ambient temperature. Ether was added and the reaction was diluted with water (200 mL). The aqueous layer was separated and extracted with ether (2×300 mL). The combined organic extracts were dried over sodium sulfate, filtered and concentrated in vacuo to afford crude product. The crude product was loaded onto a 4 inch X 6 inch silica gel column. Using 40% ether in hexane as an eluant afforded 33.63 g (96%) of (2-bromo-5-methoxy-benzyloxy)-tert-butyl-dimethyl-silane (14).

¹H NMR (CDCl₃): 0.12 (s-3H), 0.12 (s-3H), 0.96 (s-9H), 3.78 (s-3H), 4.67 (s-2H), 6.64 (m-1H), 7.14 (d-1H; J=3.11 Hz), 7.34 (d-1H; J=8.71 Hz).

### Preparation 11

### Tert-butyl-dimethyl-(2-vinyl-5-methoxy-benzyloxy)-silane (15)

Under a nitrogen atmosphere, 33.63 g (0.10 mol) of (2-bromo-5-methoxy-benzyloxy)-tert-butyl-dimethyl-silane (14), 32.18 g (0.10 mol) of tributylvinyl tin and 4.7 g (0.004 mol) of tetrakis(triphenylphosphine)palladium(0) were combined in 250 mL of toluene, and the solution was heated to reflux for 6 hours. The reaction was allowed to cool to ambient temperature and was quenched with 5% NH₄OH (2×100 mL). The organic layer was washed with water (1×200 mL) and brine (1×200 mL), dried over sodium sulfate, filtered and concentrated in vacuo to yield crude product. The crude product was chromatographed on a silica gel column, first with hexane, then with 20% CHCl₃ in hexane, and finally with 40% CHCl₃ in hexane to afford 25.0 g (89%) of tert-butyl-dimethyl-(2-vinyl-5-methoxy-benzyloxy)-silane (15).

¹H NMR (CDCl₃): 0.10 (s-6H), 0.95 (s-9H), 3.81 (s-3H), 4.77 (s-2H), 5.18 (dd-1H; J=11.0, 1.45 Hz), 5.52 (dd-1H; J=17.45, 1.45 Hz), 6.77 (m-2H), 7.05 (d-1H; J=2.70 Hz), 7.40 (d-1H; J=8.51 Hz).

### Preparation 12

### Tert-butyl-dimethyl-(2-ethyl-5-methoxy-benzyloxy)-silane (16)

25.0 g (0.0899 mol) of tert-butyl-dimethyl-(2-vinyl-5-methoxy-benzyloxy)-silane (15) was dissolved in 100 mL of ethyl acetate and was placed in a 1 L Parr Shaker bottle. 1.93 g (0.0084 mol) of catalyst (PtO₂) was added and the solution was placed under 30 PSI of hydrogen for 25 minutes. The reaction mixture was filtered through a pad of celite and concentrated in vacuo to yield crude product. The crude product was chromatographed on a silica gel column with 40% CHCl₃ in hexane to afford 24.85 g (99%) of tert-butyl-dimethyl-(2-ethyl-5-methoxy-benzyloxy)-silane (16).

¹H NMR (CDCl₃): 0.11 (s-6H), 0.95 (s-9H), 1.18 (t-3H; J=7.68 Hz), 2.52 (m-2H), 3.79 (s-3H), 4.72 (s-2H), 6.75 (m-1H), 7.06 (m-2H).

### Preparation 13

### Tert-butyl-dimethyl-(4-bromo-2-ethyl-5-methoxy-benzyloxy)-silane (17)

Under a nitrogen atmosphere, 24.85 g (0.0885 mol) of tert-butyl-dimethyl-(2-ethyl-5-methoxy-benzyloxy)-silane (16) and 15.77 g (0.0885 mmol) of NBS were combined in 500 mL of carbon tetrachloride, followed by the addition of 100 g of silica gel 60 (EM Science). The reaction was allowed to stir for 16 hours in the absence of light. The reaction mixture was filtered and the silica gel was washed with dichloromethane. The filtrate was washed with dichloromethane (1×300 mL). The combined organic extracts were washed with 1 M NaOH (2×300 mL), dilute NaHSO₃ (1×200 mL) and brine (1×200 mL), dried over magnesium sulfate, filtered and concentrated in vacuo to yield crude tert-butyl-dimethyl-(4-bromo-2-ethyl-5-methoxy-benzyloxy)-silane (17). Crude tert-butyl-dimethyl-(4-bromo-2-ethyl-5-methoxy-benzyloxy)-silane (17) was chromatographed on a silica gel column with 20% CHCl₃ in hexane to afford 19.70 g (62%) of tert-butyl-dimethyl-(4-bromo-2-ethyl-5-methoxy-benzyloxy)-silane (17).

¹H NMR (CDCl₃): 0.10 (s-6H), 0.94 (s-9H), 1.16 (t-3H; J=7.68 Hz), 2.47(m-2H), 3.87 (s-3H), 4.67 (s-2H), 7.09 (s-1H), 7.29 (s-1H).

### Preparation 14

### Tert-butyl-dimethyl-(4-boronic acid-2-ethyl-5-methoxy-benzyloxy)-silane (18)

Under a nitrogen atmosphere, 10.00 g (0.027 mol) of tert-butyl-dimethyl-(4-bromo-2-ethyl-5-methoxy-benzyloxy)-silane (17) was added to 250 mL of anhydrous THF. The solution was cooled to −78° C., and 12.25 mL (0.031 mol) of a 2.5 M solution of butyl lithium was added dropwise while maintaining the temperature below −70° C. The reaction mixture was stirred at −78° C. for 1 hour, and then the temperature was raised to −30° C. 5.21 mL (0.031 mol) of triethyl borate was added to the reaction mixture. The reaction was allowed to warm to 23° C. over a 2 hour period and was quenched with 100 mL of saturated NH₄Cl. The pH was adjusted to 5.0 with 1M HCl, and the resultant solution was extracted with ethyl acetate (2×200 mL). The combined extracts were washed with brine (1×100 mL), dried over sodium sulfate, filtered and concentrated in vacuo to afford 9.0 g (100%) of crude tert-butyl-dimethyl-(4-boronic acid-2-ethyl-5-methoxy-benzyloxy)-silane (18) which was used directly in Preparation 14.

¹H NMR (CDCl₃): 0.12 (s-6H), 0.96 (s-9H), 1.18 (t-3H), 2.51(m-2H), 3.90 (s-3H), 4.76 (s-2H), 7.12 (s-1H), 7.25 (s-1H), 7.57 (s-1H).

### Preparation 15

### 2-(4-tert-butyldimethylsilyloxymethyl-5-ethyl-2-methoxy-phenyl)-6-(N-2,2-dimethylpropamido)-pyridine (19)

Under a nitrogen atmosphere, 6.49 g (0.025 mol) of 2-bromo-6-(N-2,2-dimethylpropamido)pyridine, 9.0 g (0.027 mol) of tert-butyl-dimethyl-(4-boronic acid-2-ethyl-5-methoxy-benzyloxy)-silane (18), 10.6 g (0.10 mol) of sodium carbonate and 5.85 g (0.005 mol) of tetrakis(triphenylphosphine)palladium(0) were combined in 180 mL of ethanol and 20 mL of water. The solution was allowed to reflux for 18 hours. The reaction mixture was then concentrated in vacuo. The resultant yellow residue was partitioned between ethyl acetate (200 mL) and water (200 mL). The aqueous layer was extracted again with ethyl acetate (200 mL). The combined organic extracts were washed with brine (1×200 mL), dried over sodium sulfate, filtered and concentrated in vacuo to yield crude 2-(4-tert-butyldimethylsilyloxymethyl-5-ethyl-2-methoxy-phenyl)-6-(N-2,2-dimethylpropamido)-pyridine (19). The crude 2-(4-tert-butyldimethylsilyloxymethyl-5-ethyl-2-methoxy-phenyl)-6-(N-2,2-dimethylpropamido)-pyridine (19) was chromatographed on a silica gel column with 20% ether in hexane to afford 13.93 g of still crude 2-(4-tert-butyldimethylsilyloxymethyl-5-ethyl-2-methoxy-phenyl)-6-(N-2,2-dimethylpropamido)-pyridine (19) which was used directly in Preparation 15.

¹H NMR (CDCl₃): 0.13 (s-6H), 0.96 (s-9H), 1.21 (t-3H; J=7.48 Hz), 1.32 (s-9H), 2.57(m-2H), 3.83 (s-3H), 4.78 (s-2H), 7.19 (s-1H), 7.46 (s-1H), 7.52 (m-1H), 7.68 (t-1H), 8.07 (bs-1H), 8.12 (dd-1H; J=0.83, 8.10 Hz).

### Preparation 16

### 2-(4-hydroxymethyl-5-ethyl-2-methox(-phenyl)-6-(N-2,2-dimethylpropamido)-pyridine (20)

Under a nitrogen atmosphere, 76.34 mL (0.76 mol) of 1 M TBAF in THF was added to a THF (100 mL) solution containing 13.93 g of crude 2-(4-tert-butyldimethylsilyloxymethyl-5-ethyl-2-methoxy-phenyl)-6-(N-2,2-dimethylpropamido)-pyridine (19). The solution was allowed to stir for 18 hours. The reaction mixture was then concentrated in vacuo. The resultant residue was partitioned between ether (200 mL) and water (200 mL). The aqueous layer was extracted again with ether (200 mL). The combined organic extracts were washed with water (1×100 mL) and brine (1×100 mL), dried over sodium sulfate, filtered and concentrated in vacuo to yield 3.88 g (35% for three steps) of 2-(4-hydroxymethyl-5-ethyl-2-methoxy-phenyl)-6-(N-2,2-dimethylpropamido)-pyridine (20) as a white semisolid. The 2-(4-hydroxymethyl-5-ethyl-2-methoxy-phenyl)-6-(N-2,2-dimethylpropamido)-pyridine (20) was washed with ether and dried.

¹H NMR (CDCl₃): 1.19 (t-3H), 1.35 (s-9H), 2.60 (q-2H; J=7.47 Hz), 3.12 (t-1H; J=6.22 Hz), 3.67 (s-3H), 4.73 (m-2H), 6.90 (s-1H), 7.36 (s-1H), 7.41 (dd-1H; J=0.83, 7.47 Hz), 7.70 (t-1H; J=8.09 Hz), 8.22 (dd-1H; J=0.83, 8.30 Hz), 8.52 (s-1H).

### Preparation 17

### 2-(4-formyl-5-ethyl-2-methoxy-phenyl)-6-(N-2,2-dimethylpropamido)-pyridine (21)

Under a nitrogen atmosphere, 1.98 g (2.28 mmol) of MnO₂ was added to a toluene (50 mL) solution containing 1.56 g (4.56 mmol) of 2-(4-hydroxymethyl-5-ethyl-2-methoxy-phenyl)-6-(N-2,2-dimethylpropamido)-pyridine (20). The solution was allowed to stir for 18 hours at 90° C. The reaction mixture was then cooled and concentrated in vacuo. The resultant residue was partitioned between ethyl acetate (200 mL) and water (200 mL). The aqueous layer was extracted again with ethyl acetate (2×200 mL). The combined organic extracts were dried over sodium sulfate, filtered and concentrated in vacuo to yield crude product. The crude product was chromatographed on a silica gel column with 20% ether in hexane followed by 50% ether in hexane to afford 1.29 g (83%) of 2-(4-formyl-5-ethyl-2-methoxy-phenyl)-6-(N-2,2-dimethylpropamido)-pyridine (21).

¹H NMR (CDCl₃): 1.19 (t-3H), 1.34 (s-9H), 2.60 (q-2H; J=7.48 Hz), 3.89 (s-3H), 7.46 (s-1H), 7.56 (dd-1H; J=0.83, 7.69 Hz), 7.63 (s-1H), 7.73 (t-1H; J=7.69 Hz), 8.05 (bs-1H), 8.22 (dd-1H; J=0.83, 8.30 Hz), 10.35 (s-1H).

### Preparation 18

### 2-(4-N,N-dimethylaminomethyl-5-ethyl-2-methoxy-phenyl)-6-(N-2,2-dimethylpropamido)-pyridine (22)

Under a nitrogen atmosphere, 330 mg (0.97 mmol) of 2-(4-formyl-5-ethyl-2-methoxy-phenyl)-6-(N-2,2-dimethylpropamido)-pyridine (21), 3 mL (6.00 mmol) of 2M N,N-dimethylamine in THF, 390 mg (1.84 mmol) of sodium triacetoxyborohydride and 120 uL (1.94 mmol) of acetic acid were combined in dichloromethane (5 mL). The solution was allowed to stir for 5 hours at 23° C. The reaction mixture was then washed with 1 M NaOH. The aqueous layer was extracted again with dichloromethane (2×50 mL). The combined organic extracts were dried over sodium sulfate, filtered and concentrated in vacuo to yield crude product. The crude product was chromatographed on a silica gel column with 10% methanol in dichloromethane to afford 367 mg (100%) of 2-(4-N,N-dimethylaminomethyl-5-ethyl-2-methoxy-phenyl)-6-(N-2,2-dimethylpropamido)-pyridine (22).

¹H NMR (CDCl₃): 1.20 (t-3H), 1.31 (s-9H), 2.30 (s-6H), 2.69 (q-2H; J=7.48 Hz), 3.48 (s-2H), 3.83 (s-3H), 7.08 (s-1H), 7.48-7.52 (m-2H), 7.69 (t-1H), 8.10 (bs-1H), 8.15 (d-1H; J=8.30 Hz).

### EXAMPLE 3

### 6-[4-(N,N-dimethylaminomethyl)-5-ethyl-2-methoxy-phenyl]-pyridin-2-ylamine (23)

Under a nitrogen atmosphere, 367 mg (0.97 mmol) of 2-(4-N,N-dimethylaminomethyl-5-ethyl-2-methoxy-phenyl)-6-(N-2,2-dimethylpropamido)-pyridine (22) and 10 mL of 6N HCl were combined in 10 mL of dioxane. The reaction was allowed to reflux with stirring for 16 hours, allowed to cool to ambient temperature, and diluted with 1M NaOH until the solution was basic. The resultant solution was extracted with dichloromethane (3×50 mL). The combined organic extracts were dried over sodium sulfate, filtered and concentrated in vacuo to yield crude product. The crude product was chromatographed on a silica gel column with 80% ethyl acetate in hexane followed by 10% methanol in dichloromethane to afford 137 mg (49%) of 6-[4-(N,N-dimethylaminomethyl)-5-ethyl-2-methoxy-phenyl]-pyridin-2-ylamine (23).

¹H NMR (CDCl₃): 1.19 (t-3H), 2.25 (s-6H), 2.66 (q-2H; J=7.68 Hz), 3.41 (s-2H), 3.82 (s-3H), 4.41 (bs-2H), 6.42 (d-1H; J=8.10 Hz), 6.99 (s-1H), 7.14 (d-1H; J=7.48 Hz), 7.43-7.49 (m-2H).

### Preparation 19

### 2-(4-N-methylaminomethyl-5-ethyl-2-methoxy-phenyl)-6-(N-2,2-dimethylpropamido)-pyridine (24)

Under a nitrogen atmosphere, 830 mg (2.44 mmol) of 2-(4-formyl-5-ethyl-2-methoxy-phenyl)-6-(N-2,2-dimethylpropamido)-pyridine (21), 12.2 mL (24.4 mmol) of 2M N-methylamine in THF and 979 mg (4.62 mmol) of sodium triacetoxyborohydride were combined in dichloromethane (25 mL). The resultant solution was stirred for 12 hours. 1.82 mL (32.9 mmol) of acetic acid was added, followed by another 979 mg (4.62 mmol) of sodium triacetoxyborohydride. The solution was allowed to stir for an additional 12 hours at 23° C. The reaction mixture was then washed with 1N NaOH. The aqueous layer was extracted again with dichloromethane (3×50 mL). The combined organic extracts were dried over sodium sulfate, filtered and concentrated in vacuo to yield crude product. The crude product was chromatographed on a silica gel column with 5-10% methanol in dichloromethane to afford 551 mg (64%) of 2-(4-N-methylaminomethyl-5-ethyl-2-methoxy-phenyl)-6-(N-2,2-dimethylpropamido)-pyridine (24).

¹H NMR (CDCl₃): 1.23 (t-3H; J=7.48 Hz), 1.32 (s-9H), 1.56 (bs-1H), 2.52 (s-3H), 2.69 (q-2H; J=7.48 Hz), 3.79 (s-2H), 3.83 (s-3H), 7.03 (s-1H), 7.49-7.51 (m-2H), 7.69 (t-1H; J=7.89 Hz), 8.05 (bs-1H), 8.15 (d-1H; J=7.89 Hz).

### EXAMPLE 4

### 6-[4-(N-methylaminomethyl)-5-ethyl-2-methoxy-phenyl]-pyridin-2-ylamine (25)

Under a nitrogen atmosphere, 550 mg (1.55 mmol) of 2-(4-N-methylaminomethyl-5-ethyl-2-methoxy-phenyl)-6-(N-2,2-dimethylpropamido)-pyridine (24) and 30 mL of 6N HCl were combined in 30 mL of dioxane. The reaction was allowed to reflux with stirring for 48 hours, allowed to cool to ambient temperature, and diluted with 1N NaOH until the solution was basic. The resultant solution was extracted with dichloromethane (3×50 mL). The combined organic extracts were dried over sodium sulfate, filtered and concentrated in vacuo to yield crude product. The crude product was chromatographed on a silica gel column with 80% ethyl acetate in hexane, followed by 10%-15% methanol in dichloromethane, to afford 290 mg (69%) of 6-[4-(N-methylaminomethyl)-5-ethyl-2-methoxy-phenyl]-pyridin-2-ylamine (25).

¹H NMR (CDCl₃): 1.19 (t-3H; J=7.47 Hz), 1.80 (bs-1H), 2.50 (s-3H), 2.65 (q-2H; J=7.47 Hz), 3.77 (s-2H), 3.82 (s-3H), 4.44 (bs-2H), 6.42 (d-1H; J=8.30 Hz), 6.99 (s-1H), 7.13 (d-1H; J=7.89 Hz), 7.44 (t-1H; J=7.89 Hz), 7.51 (s-1H).

The following compounds were made from Preparation 17 (21) according to the same procedure as was Example 4:

| | |
|---|---|
| Example 4(a) | 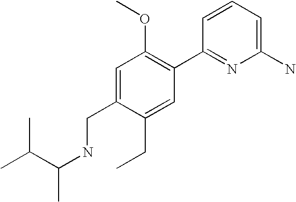 |
| Example 4(b) | 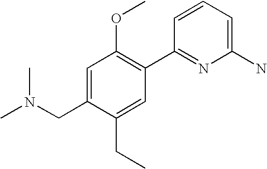 |
| Example 4(c) | 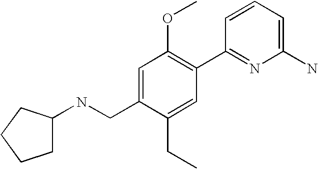 |
| Example 4(d) | 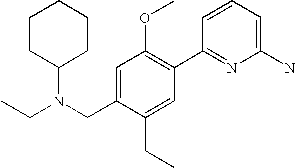 |
| Example 4(e) | 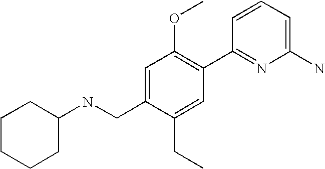 |
| Example 4(f) | 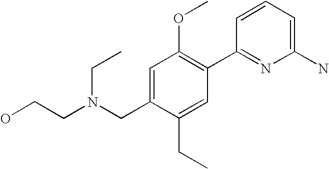 |
| Example 4(g) | 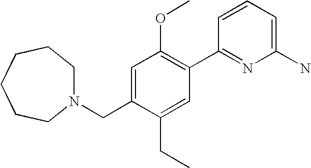 |
| Example 4(h) | 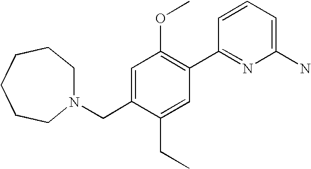 |
| Example 4(i) | 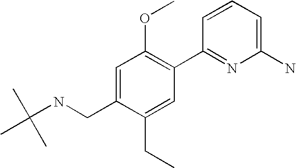 |
| Example 4(j) | 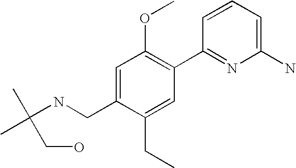 |
| Example 4(k) | 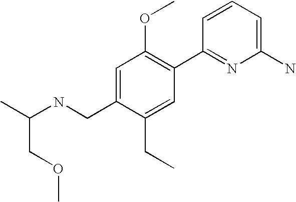 |
| Example 4(l) | 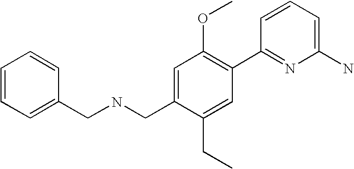 |
| Example 4(m) | 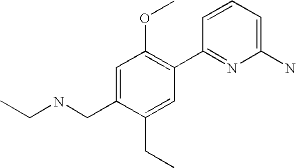 |
| Example 4(n) | 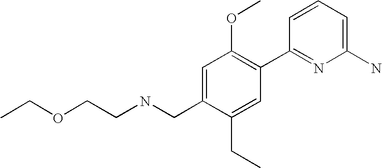 |
| Example 4(o) | 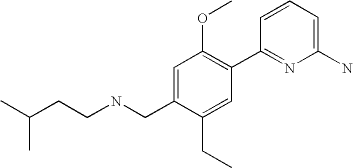 |
| Example 4(p) | 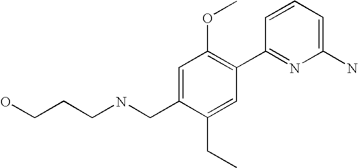 |
| Example 4(q) | 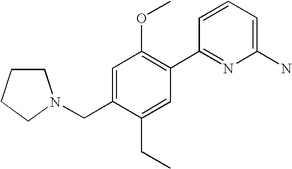 |
| Example 4(r) | 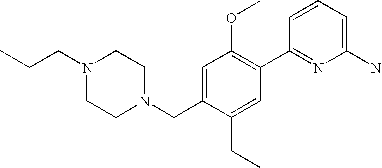 |
| Example 4(s) | 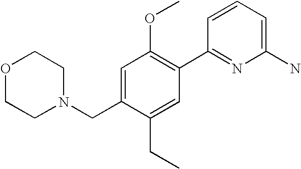 |
| Example 4(t) | 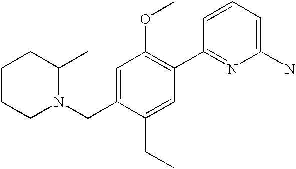 |
| Example 4(u) | 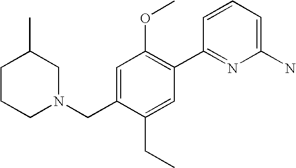 |
| Example 4(v) | 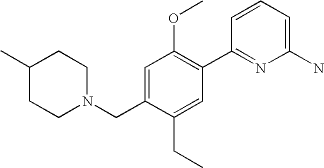 |
| Example 4(w) | 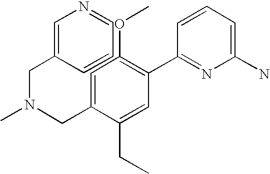 |
| Example 4(x) | 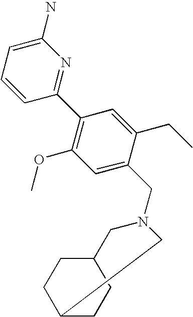 |
| Example 4(y) | 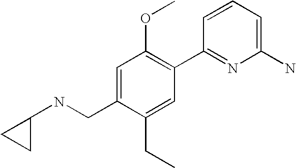 |
| Example 4(z) | 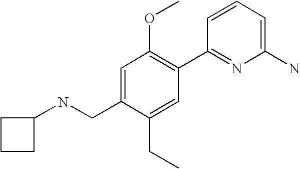 |
| Example 4(aa) | 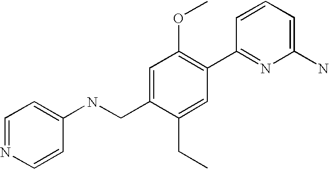 |
| Example 4(bb) | 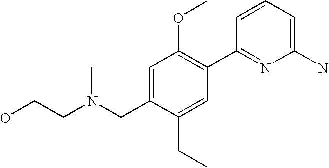 |
| Example 4(cc) | 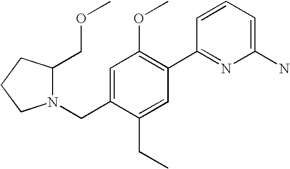 |
| Example 4(dd) | 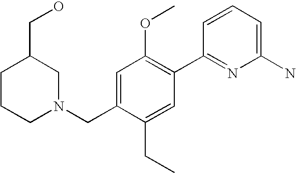 |
| Example 4(ee) | 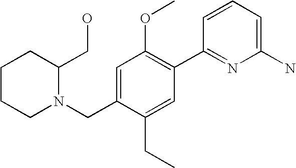 |
| Example 4(ff) | 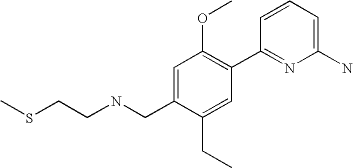 |
| Example 4(gg) | 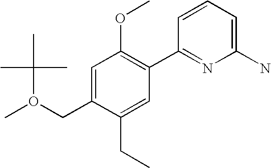 |
| Example 4(hh) | 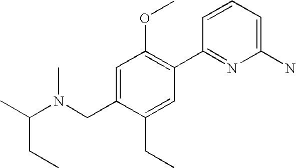 |
| Example 4(ii) | 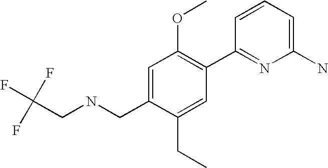 |
| Example 4(jj) | 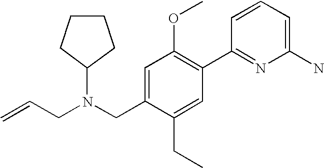 |
| Example 4(kk) | 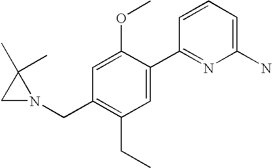 |
| Example 4(ll) | 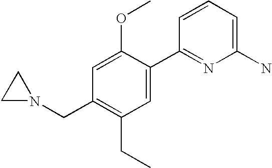 |
| Example 4(mm) | 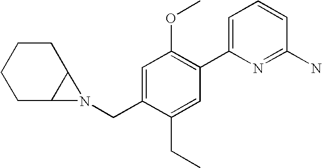 |
| Example 4(nn) | 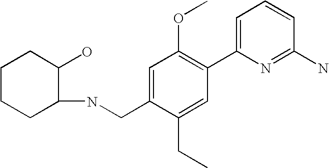 |
| Example 4(oo) | 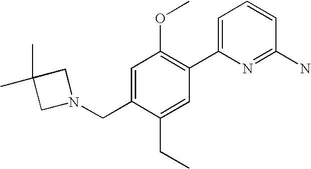 |

### Preparation 20

### 2-(4-bromo-2,5-dimethoxy-phenyl)-[1,3]dioxolane (26)

Under a N₂ atmosphere in 500 mL of benzene was combined 11.4 g (0.0465 mol) of 4-bromo-2,5-dimethoxy benzaldehyde and 35.80 g/32.2 ml (0.5768 mol) of ethylene glycol followed by 797 mg (0.0042 mol) of p-toluenesulfonic acid mono hydrate. The reaction was allowed to reflux using a Dean-Stark apparatus for 5.75 h. Water (1.75 ml) was collected. The reaction mixture was washed with with with 1N NaOH (1×200 mL) and brine (3×125 mL), dried over sodium sulfate, filtered and concentrated in vacuo to yield 14.38 g (>100%) of crude product 26.

¹H NMR (CDCl₃): 3.81 (s-3H), 3.86 (s-3H), 4.00-4.15 (m-4H), 6.04 (s-1H), 7.09 (s-1H), 7.13 (s-1H).

### Preparation 21

### 2-(4-boronic Acid-2,5-dimethoxy-phenyl)-[1,3]dioxolane (27)

Under a N₂ atmosphere in 80 mL of anhydrous THF was added 12.63 g (0.0437 mol) of 2-(4-bromo-2,5-dimethoxy-phenyl)-[1,3]dioxolane 26. The solution was cooled to −78° C. and 19.22 mL (0.0481 mol) of a 2.5 M solution of butyl lithium was added dropwise while maintaining the temperature below −70° C. The reaction mixture was stirred at −78° C. for 0.5 hr at which point 7.01 g/8.17 mL (0.0481 mol) of triethyl borate was added. The reaction was allowed to warm to 23° C. over a 2 hr period and was quenched with 125 mL of saturated NH₄Cl, the pH was adjusted to 5.0 with 1 M HCl and the resultant solution was extracted with ethyl acetate (2×200 mL). The combined extracts were washed with brine (1×100 mL), dried over sodium sulfate, filtered and concentrated in vacuo to afford 8.37 g (75%) of crude product 27 which was used directly in the next reaction.

### Preparation 22

### N-[6-(4-[1,3] Dioxolan-2-yl-2,5-dimethoxy-phenyl)-pyridin-2-yl]2,2-dimethylpropionamide (28)

Under a nitrogen atmosphere was combined 8.37 g (0.03295 mol) of 2-(4-boronic acid-2,5-dimethoxy-phenyl)-[1,3]dioxolane 27, 7.52 g (0.02995 mol) of 2-bromo-6-(2,5-dimethyl-pyrrol-1-yl)-pyridine, 12.70 g (0.120 mol) of sodium carbonate and 752 mg (0.00047 mol) of tetrakis(triphenylphosphine)palladium(0) in 135 mL of ethanol and 15 mL of water. The solution was allowed to reflux for 18 hrs at which point the reaction mixture was concentrated in vacuo. The resultant yellow residue was partitioned between ethyl acetate (200 mL) and water (200 mL). The aqueous layer was extracted again with ethyl acetate (200 mL) and the combined organic extracts were washed with brine (1×200 mL), dried over sodium sulfate, filtered and concentrated in vacuo to yield crude product which was chromatographed on a silica gel column with 5 to 15% ethyl acetate in hexane to afford 10.88 g of crude 28 which was taken into the next step.

¹H NMR (CDCl₃): 2.22 (s-6H), 3.87 (s-3H), 3.88 (s-3H), 4.03-4.19 (m-4H), 5.91 (s-2H), 6.13 (s-1H), 7.10 (dd-1H, J=0.82, 7.74 Hz), 7.24 (d-1H, J=0.82 Hz), 7.57 (s-1H), 7.81 (t-1H), 8.00 (d-1H, J=0.8 Hz).

### Preparation 23

### [6-(2,5-dimethyl-pyrrol-1-yl)-pyridin-2-yl]-2,5-dimethoxy-benzaldehyde (29)

Under a nitrogen atmosphere in a THF (50 mL) solution containing 5.00 g (01314 mol) of N-[6-(4-[1,3] Dioxolan-2-yl-2,5-dimethoxy-phenyl)-pyridin-2-yl]2,2-dimethylpropionamide 28 was added 25 mL (0.0375 mol) of 1.5 M aqueous HOAc in addition to 25 ml of glacial HOAc. The solution was allowed to stir for 3 hrs at which point the reaction partitioned between dichloromethane (200 mL) and 1N NaOH (200 mL). The aqueous layer was extracted again with dichloromethane (200 mL) and the combined organic extracts were dried over sodium sulfate, filtered and concentrated in vacuo to yield 4.65 g of crude which was chromatographed on a silica gel column with 10 to 15% ethyl acetate in hexane to afford 2.99 g (68%) of 29.

¹H NMR (CDCl₃): 2.22 (s-6H), 3.91 (s-3H), 3.93 (s-3H), 5.92 (s-2H), 7.17 (dd-1H, J=0.82, 7.68 Hz), 7.45 (s-1H), 7.67 (s-1H), 7.86 (t-1H), 8.05 (d-1H, J=0.82, 7.88 Hz), 10.47 (s-1H).

### Preparation 24

### 4-[6-(2,5-dimethyl-pyrrol-1-yl)-pyridin-2-yl]-2,5-dimethoxy-benzyl]-dimethylamine (30)

Under a nitrogen atmosphere in dichloroethane (5 mL) was combined 200 mg (0.595 mmol) of N-[6-(2,5-dimethyl-pyrrol-1-yl)-pyridin-2-yl]-2,5-dimethoxy-benzaldehyde 29, 327 ul (0.654 mmol) of 2M N,N-dimethylamine in THF, 239 mg (1.13 mmol) of sodium triacetoxyborohydride and 68 uL (1.19 mmol) of acetic acid. The solution was allowed to stir for 5 hrs at 23° C. which point the reaction mixture was washed with 1 M NaOH and then the aqueous layer was extracted again with dichloromethane (2×50 mL) and the combined organic extracts were dried over sodium sulfate, filtered and concentrated in vacuo to yield crude product which was chromatographed on a silica gel column with 50 to 80% ethyl acetate in hexane to afford 183 mg (84%) of 4-[6-(2,5-dimethyl-pyrrol-1-yl)-pyridin-2-yl]-2,5-dimethoxy-benzyl]-dimethylamine 30.

¹H NMR (CDCl₃): 2.22 (s-6H), 2.28 (s-6H), 3.48 (s-2H), 3.82 (s-3H), 3.87 (s-3H), 5.89 (s-2H), 7.03 (dd-1H), 7.08 (dd-1H),7.52 (s-1H), 7.86 (t-1H), 8.00 (d-1H).

### EXAMPLE 5

### 6-(4-dimethylaminomethyl-2,5-dimethoxy-phenyl]-pyridine-2-ylamine (31)

Under a N₂ atmosphere in 16 mL of 6:1 ethanol:water was combined 180 mg (0.493 mmol) of 4-[6-(2,5-dimethyl-pyrrol-1-yl)-pyridin-2-yl]-2,5-dimethoxy-benzyl]-dimethylamine 30 and 411 mg (5.92 mmol) of hydroxylamine HCl. The reaction was heated at 90° C. with stirring for 16 hrs and was allowed to cool to ambient temperature and was diluted with saturated sodium bicarbonate. The resultant solution was extracted a with dichloromethane (3×50 mL) and the combined organic extracts were dried over sodium sulfate, filtered and concentrated in vacuo to yield crude product which was chromatographed on a silica gel column with 2 to 10% methanol in dichloromethane in hexane to afford 105 mg (74%) of 6-(4-dimethylaminomethyl-2,5-dimethoxy-phenyl]-pyridine-2-ylamine 31.

¹H NMR (CDCl₃): 2.27 (s-6H), 3.47 (s-2H), 3.79 (s-3H), 3.83 (s-3H), 4.42 (bs-2H), 6.42 (dd-1H, J=0.82, 8.07 Hz), 6.99 (s-1H), 7.20 (dd-1H, J=0.82, 7.57 Hz), 7.27 (s-1H), 7.45 (t-1H).

The following compounds were synthesized according to the same procedure used for the conversion of (29) to (31) described above:

| | |
|---|---|
| Example 5(a) | 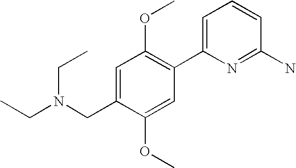 |
| Example 5(b) | 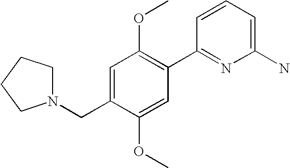 |
| Example 5(c) | 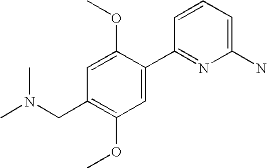 |
| Example 5(d) | 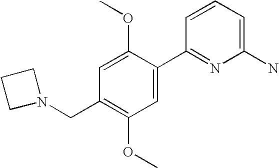 |
| Example 5(e) | 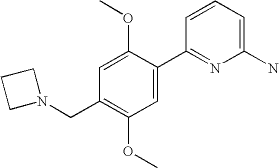 |
| Example 5(f) | 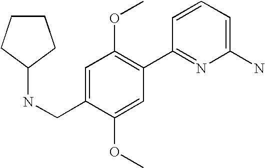 |
| Example 5(g) | 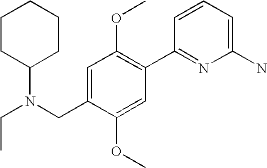 |
| Example 5(h) | 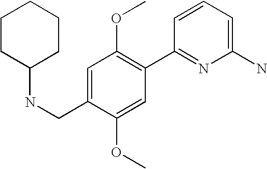 |
| Example 5(i) | 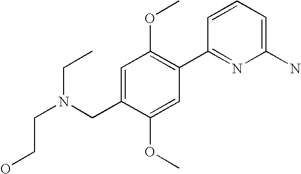 |
| Example 5(j) | 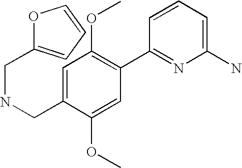 |
| Example 5(k) | 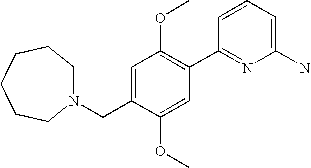 |
| Example 5(l) | 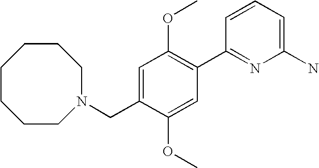 |
| Example 5(m) | 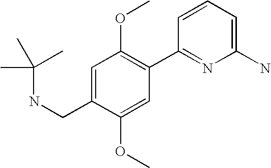 |
| Example 5(n) | 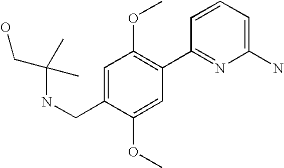 |
| Example 5(o) | 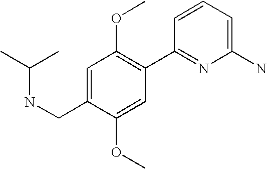 |
| Example 5(p) | 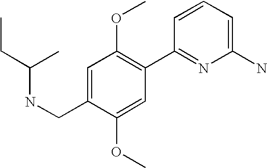 |
| Example 5(q) | 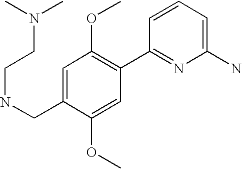 |
| Example 5(r) | 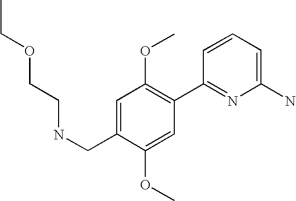 |
| Example 5(s) | 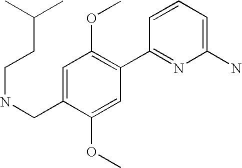 |
| Example 5(t) | 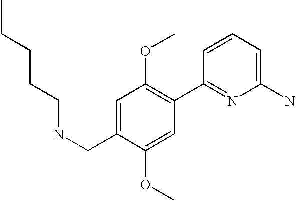 |
| Example 5(u) | 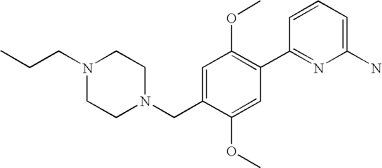 |
| Example 5(v) | 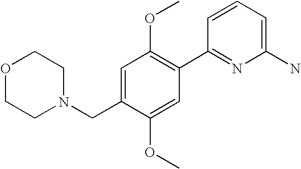 |
| Example 5(w) | 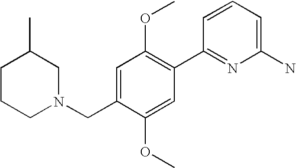 |
| Example 5(x) | 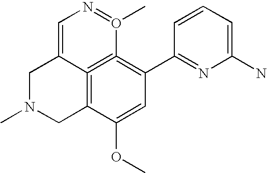 |
| Example 5(y) | 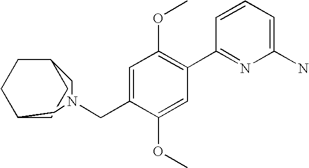 |
| Example 5(z) | 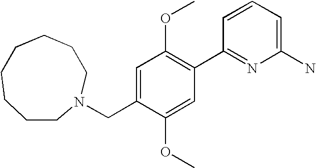 |
| Example 5(aa) | 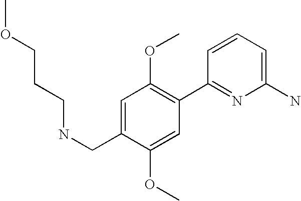 |
| Example 5(bb) | 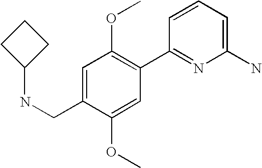 |
| Example 5(cc) | 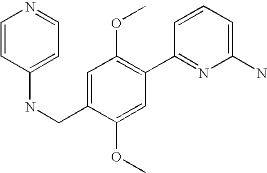 |
| Example 5(dd) | 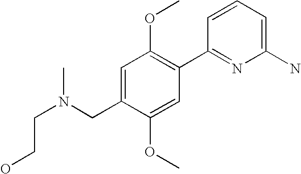 |
| Example 5(ee) | 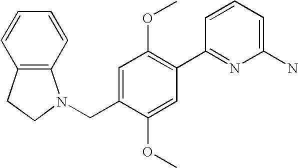 |
| Example 5(ff) | 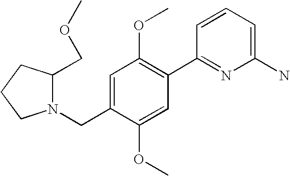 |
| Example 5(gg) | 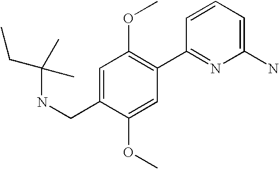 |
| Example 5(hh) | 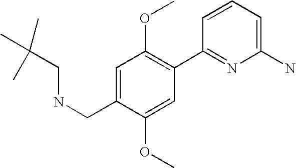 |
| Example 5(ii) | 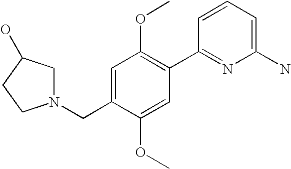 |
| Example 5(jj) | 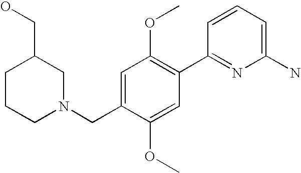 |
| Example 5(kk) | 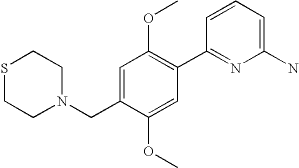 |
| Example 5(ll) | 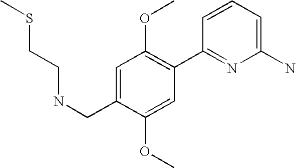 |
| Example 5(mm) | 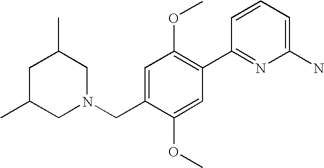 |
| Example 5(nn) | 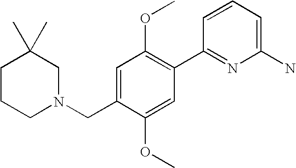 |
| Example 5(oo) | 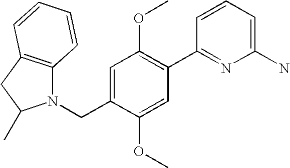 |
| Example 5(pp) | 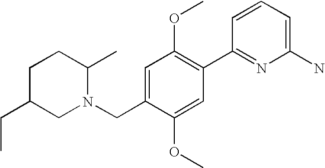 |
| Example 5(qq) | 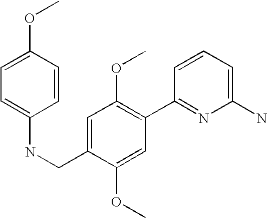 |
| Example 5(rr) | 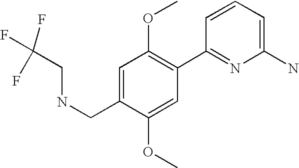 |
| Example 5(ss) | 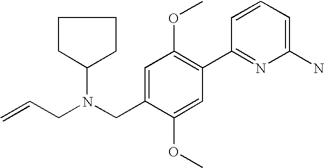 |
| Example 5(tt) | 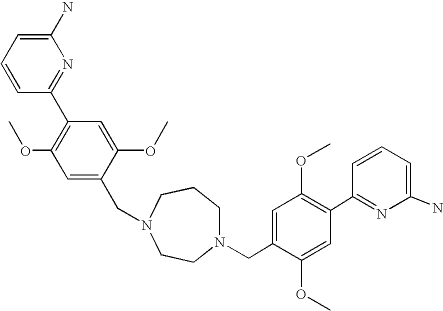 |
| Example 5(uu) | 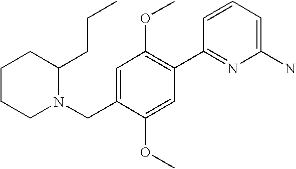 |
| Example 5(vv) | 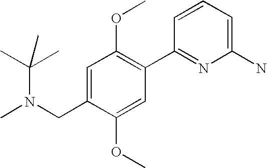 |
| Example 5(ww) | 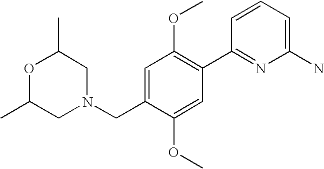 |
| Example 5(xx) | 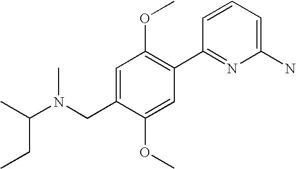 |
| Example 5(yy) | 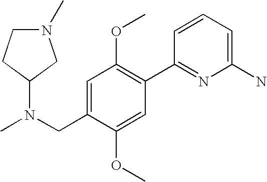 |
| Example 5(zz) | 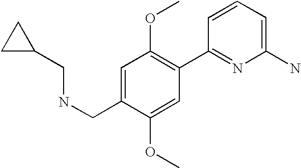 |
| Example 5(aaa) | 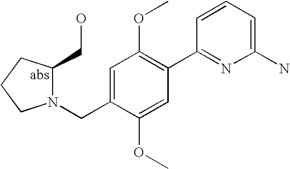 |
| Example 5(bbb) | 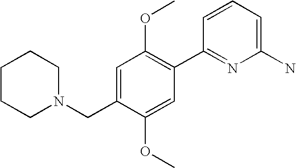 |
| Example 5(ccc) | 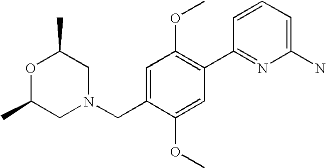 |
| Example 5(ddd) | 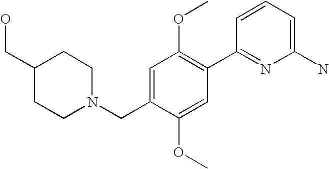 |
| Example 5(eee) | 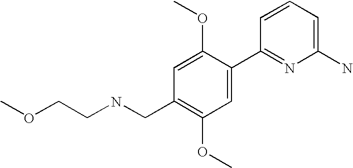 |
| Example 5(fff) | 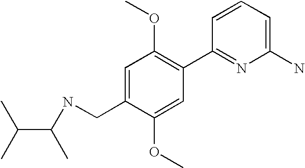 |
| Example 5(ggg) | 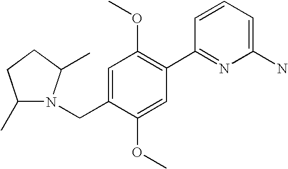 |
| Example 5(hhh) | 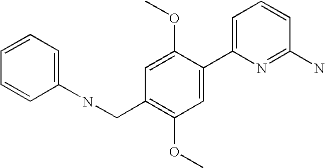 |
| Example 5(iii) | 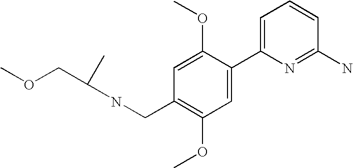 |
| Example 5(jjj) | 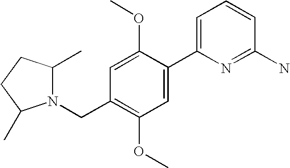 |
| Example 5(kkk) | 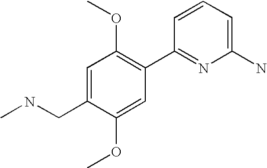 |

### Preparation 25

### (3-{4-[6-(2,5-dimethyl-pyrrol-1-yl)-pyridin-2-yl]-2,5-dimethoxy-phenyl}-allyl)-dimethylamine (32)

Under a nitrogen atmosphere in anhydrous THF (3 mL) containing 308 mg (0.744 mmol) of 2-dimethylaminoethyl triphenylphosphonium bromide was added 818 ul (818 mmol) of 1M lithium bis (trimethylsilyl)amide. The reaction was stirred at room tempoerature for 15 min at which point 200 mg (0.595 mmol) of N-[6-(2,5-dimethyl-pyrrol-1-yl)-pyridin-2-yl]-2,5-dimethoxy-benzaldehyde was added. The reaction mixture was stirred for an additional 30 minutes at which point dichloromethane was added and the reaction mixture was washed with water followed by brine and then the aqueous layers were extracted again with dichloromethane (2×50 mL) and the combined organic extracts were dried over sodium sulfate, filtered and concentrated in vacuo to yield crude product which was chromatographed on a silica gel column with 50 to 90% ethyl acetate in hexane followed by 10% methanol in dichloromethane to afford 91 mg (39%) of (3-{4-[6-(2,5-dimethyl-pyrrol-1-yl)]-pyridin-2-yl)-2,5-dimethoxy-phenyl}-allyl)-dimethylamine 32.

¹H NMR (CDCl₃): 2.22 (s-6H), 2.31 (s-6H), 3.13 (m-2H), 3.84 (s-3H), 3.87 (s-3H), 5.90 (s-2H), 6.31-6.38(m-1H), 6.86 (d-1H, J=4.9 Hz), 7.07-7.11 (m-2H), 7.57 (s-1H), 7.80 (t-1H, J=7.89 Hz), 8.01 (d-1H, J=7.68 Hz).

### Preparation 26

### (3-{4-[6-(2,5-dimethyl-pyrrol-1-yl)-pyridin-2-yl]-2,5-dimethoxy-phenyl}-propyl)-dimethylamine (33)

In a flame dried 250 ml Paar bottle was combined 89 mg (227 mmol) of (3-{4-[6-(2,5-dimethyl-pyrrol-1-yl)-pyridin-2-yl]-2,5-dimethoxy-phenyl}-allyl)-dimethylamine in 5 mL of absolute ethanol 10% Pd/C (10 mg) was added and the reaction mixture was treated under 37 PSI hydrogen for 1 h. Additional Pd/C (2×10 mg) was added and the reaction mixture was treated with hydrogen (50 PSI) for a total of 5 h. The reaction mixture was filtered through celite, washed with ethanol and concentrated in vacuo to yield crude product which was chromatographed on a silica gel column with 0 to 5% methanol in dichloromethane to afford 25 mg (28%) of (3-{4-[6-(2,5-dimethyl-pyrrol-1-yl)-pyridin-2-yl]-2,5-dimethoxy-phenyl}-propyl)-dimethylamine 33.

¹H NMR (CDCl₃): 1.92-1.99 (m-2H), 2.21 (s-6H), 2.45 (s-6H), 2.57-2.62 (m-2H), 2.67-2.72 (m-2H), 3.80 (s-3H), 3.85 (s-3H), 5.89 (s-2H), 6.83 (s-1H), 7.07 (d-1H, J=7.74 Hz), 7.48 (s-1H), 7.80 (t-1H), 7.97 (d-1H, J=7.74 Hz).

### EXAMPLE 6

### 6-[4-(3-dimethylamino-propyl-2,5-dimethoxy-phenyl]-pyridin-2-ylamine (34)

Under a N₂ atmosphere in 3.5 mL of 6:1 ethanol:water was combined 25 mg (0.0635 mmol) of (3-{4-[6-(2,5-dimethyl-pyrrol-1-yl)-pyridin-2-yl]-2,5-dimethoxy-phenyl}-propyl)-dimethylamine 33 and 53 mg (0.762 mmol) of hydroxylamine HCl. The reaction was heated at 90° C. with stirring for 2 hrs. Additional 53 mg (0.762 mmol) of hydroxylamine HCl was added and the reaction mixture was heated at 90° C. with stirring for an additional 2 hrs was allowed to cool to ambient temperature for 5 days and was diluted with saturated sodium bicarbonate. The resultant solution was extracted a with dichloromethane (3×50 mL) and the combined organic extracts were dried over sodium sulfate, filtered and concentrated in vacuo to yield crude product which was chromatographed on a silica gel column with 0 to 15 followed by 2% NH₄OH/10% MeOH/88% dichloromethane to afford 12 mg (60%) of 6-[4-(3-dimethylamino-propyl-2,5-dimethoxy-phenyl]-pyridin-2-ylamine 34.

¹H NMR (CDCl₃): 1.73-1.81 (m-2H), 2.22 (s-6H), 2.22-2.30 (m-2H), 2.60-2.65 (m-2H), 3.76 (s-3H), 3.81 (s-3H), 4.43 (bs-2H), 6.39 (d-1H), 6.78 (s-1H), 7.18 (dd-1H), 7.23 (d-1H, J=2.14 Hz), 7.44 (t-1H).

### Preparation 27

### {6-[4-(3-dimethylamino-propenyl)-5-ethyl-2-methoxy-phenyl]-pyridin-2-yl]-carbamic Acid Tert-Butyl Ester (35)

Under a nitrogen atmosphere in anhydrous THF (3 mL) containing 76 mg (0.184 mmol) of 2-dimethylaminoethyl triphenylphosphonium bromide was added 202 ul (0.202 mmol) of 1M lithium bis (trimethylsilyl)amide. The reaction was stirred at room tempoerature for 15 min at which point 50 mg (0.147 mmol) of [6-(5-ethyl-4-formyl-2-methoxy-phenyl)-pyridin-2-yl]-carbamic acid tert-butyl ester 9 was added. The reaction mixture was stirred for an additional 12 hours at which point dichloromethane was added and the reaction mixture was washed with water followed by brine and then the aqueous layers were extracted again with dichloromethane (2×50 mL) and the combined organic extracts were dried over sodium sulfate, filtered and concentrated in vacuo to yield crude product which was taken on to the next step.

### Preparation 28

### {6-[4-(3-dimethylamino-propyl)-5-ethyl-2-methoxy-phenyl]-pyridin-2-yl]-carbamic Acid Tert-Butyl Ester (36)

In a flame dried 250 ml Paar bottle was combined crude {6-[4-(3-dimethylamino-propenyl)-5-ethyl-2-methoxy-phenyl]-pyridin-2-yl]-carbamic acid tert-butyl ester in 10 mL of an ethanol solution. 10% Pd/C (10 mg) was added and the reaction mixture was treated under 37 PSI hydrogen for 1 h. Additional Pd/C (2×10 mg) was added and the reaction mixture was treated with hydrogen (50 PSI) for a total of 14 hours. The reaction mixture was filtered through celite, washed with ethanol and concentrated in vacuo to yield crude product which was chromatographed on a silica gel column with 0 to 10% methanol in dichloromethane to afford 36 mg (63%) of {6-[4-(3-dimethylamino-propyl)-5-ethyl-2-methoxy-phenyl]-pyridin-2-yl]-carbamic acid tert-butyl ester 36.

¹H NMR (CDCl₃): 1.21 (t-3H, J=7.47), 1.31 (s-9H), 1.81-1.85 (m-2H), 2.32 (s-6H), 2.43-2.47 (m-2H), 2.60-2.69 (m-4H), 3.80 (s-3H), 6.78 (s-1H), 7.45-7.49 (m-2H), 7.65-7.70 (t-1H), 8.10-8.15 (m-2H).

### EXAMPLE 7

### {6-[4-(3-dimethylamino-propyl)-5-ethyl-2-methoxy-phenyl]-pyridin-2-ylamine (37)

Under a N₂ atmosphere in 3.5 mL of 6:1 ethanol:water was combined 25 mg (0.0635 mmol) of {6-[4-(3-dimethylamino-propyl)-5-ethyl-2-methoxy-phenyl]-pyridin-2-yl]-carbamic acid tert-butyl ester and 53 mg (0.762 mmol) of hydroxylamine HCl. The reaction was heated at 90° C. with stirring for 2 hours. Additional 53 mg (0.762 mmol) of hydroxylamine HCl was added and the reaction mixture was heated at 90° C. with stirring for an additional 2 hrs was allowed to cool to ambient temperature for 5 days, and was diluted with saturated sodium bicarbonate. The resultant solution was extracted a with dichloromethane (3×50 mL) and the combined organic extracts were dried over sodium sulfate, filtered and concentrated in vacuo to yield crude product which was chromatographed on a silica gel column with 0 to 15% methanol in dichloromethane followed by 2% NH₄OH/10% MeOH/88% dichloromethane to afford 12 mg (60%) of {6-[4-(3-dimethylamino-propyl)-5-ethyl-2-methoxy-phenyl]-pyridin-2-ylamine 37.

¹H NMR (CDCl₃): 1.22 (t-3H), 1.74-1.82 (m-2H), 2.26 (s-6H), 2.30-2.38 (m-2H), 2.59-2.67 (m-4H), 3.79 (s-3H), 4.42 (bs-2H), 6.40 (d-1H, J=8.10 Hz), 6.75 (s-1H), 7.12 (d-1H, J=7.47 Hz), 7.41-7.48 (m-2H).

### Preparation 28

### Acetic Acid 2-(2-acetyl-5-methoxy-phenyl)-ethyl Ester (38)

28.90 g (0.217 mol) of aluminum chloride was slurried in 400 mL of methylene chloride under a N₂ atmosphere and cooled to 0° C. 15.0 g (0.0985 mol) of 2-(3-Methoxy-phenyl)-ethanol and 17.02 g (0.217 mol) of acetyl chloride were combined in methylene chloride and following the exotherm, this solution was added dropwise to the aluminum chloride slurry at 0° C. The reaction mixture was allowed to stir at 0° C. for 1 h and at ambient temperature for 45 min. The reaction was carefully poured into 1M HCl (300 ml) and the aqueous layer was extracted with dichloromethane (3×100 ml) and the combined organic extracts were dried over sodium sulfate, filtered and concentrated in vacuo to afford crude product which was loaded onto a silica gel column. Using 20% ethyl acetate in hexane yielded 17.94 g (77%) of Acetic acid 2-(2-acetyl-5-methoxy-phenyl)-ethyl ester 38.

¹H NMR (CDCl₃): 2.00 (s-3H), 2.54 (s-3H), 3.24 (t-2H), 3.84 (s-3H), 4.28 (t-2H), 6.75-6.81 (m-2H), 7.77 (d-1H; J=8.52 Hz).

### Preparation 29

### 2-(2-Ethyl-5-methoxy-phenyl)-ethanol (39)

Lithium aluminum hydride (7.2 g, 0.190 mol) was added to a solution containing 500 ml of diethyl ether at 0° C. Over a 90 min period, 50 g (0.380 mol) was added portionwise. Once complete, acetic acid 2-(2-acetyl-5-methoxy-phenyl)-ethyl ester (17.94 g, 0.076 mol) was dissolved in diethyl ether over a 2 h period and was allowed to warm to ambient temperature. The reaction mixture was allowed to stir at ambient temperature for 30 min and then heated at 55° C. for 30 min. Ethyl acetate was added and the reaction was diluted with water (7 mL), 15% NaOH (7 mL), and water (100 mL). The aqueous layer was separated and extracted with ethyl acetate (2×300 mL) and the combined organic extracts were dried over sodium sulfate, filtered and concentrated in vacuo to afford 12.9 g of crude product which was loaded onto a silica gel column. Using 20-40% ether in hexane to yield 10.80 g (79%) of 2-(2-ethyl-5-methoxy-phenyl)-ethanol 39.

¹H NMR (CDCl₃): 1.18 (t-3H), 1.60 (bs-1H), 2.59 (q-2H), 2.87 (t-2H), 3.77 (s-3H), 3.82 (t-2H), 6.72-6.75 (m-2H), 7.09-7.11 (m-1H).

### Preparation 30

### [2-(2-ethyl-5-methoxy-phenyl)-ethoxy]-dimethyl-silane (40)

10.8 g (0.0599 mol) of 2-(2-Ethyl-5-methoxy-phenyl)-ethanol was dissolved in 300 mL of anhydrous THF under a N₂ atmosphere. 8.16 g (0.120 mol) of imidazole was added followed by 9.93 g (0.066 mol) of t-butyl dimethylsilylchloride. The reaction mixture was allowed to stir at ambient temperature for 16 h. Ethyl acetate was added and the reaction was diluted with water (200 mL). The aqueous layer was separated and extracted with ethyl acetate (2×300 mL) and the combined organic extracts were dried over sodium sulfate, filtered and concentrated in vacuo to afford crude product which was loaded onto a silica gel column. Using 40% ethyl ether in hexane yielded 16.88 g (96%) of tert-Butyl-[2-(2-ethyl-5-methoxy-phenyl)-ethoxy]-dimethyl-silane 40.

¹H NMR (CDCl₃): 0.005 (s-6H), 0.88 (s-9H), 1.18 (t-3H), 2.59 (q-2H), 2.82 (t-2H), 3.76 (s-3H), 3.79 (t-2H), 6.70-6.72 (m-2H), 7.09-7.11 (m-1H).

### Preparation 31

### [2-(4-Bromo-2-ethyl-5-methoxy-phenyl)-ethoxy]-tert-butyl-dimethyl-silane (41)

DMF was dried over 4A molecular sieves. Under a N₂ atmosphere in 40 mL of dry DMF at 0° C. was added 7.86 g (0.044 mmol) of NBS followed by the dropwise addition of 13.0 (0.044 g) of tert-Butyl-[2-(2-ethyl-5-methoxy-phenyl)-ethoxy]-dimethyl-silane in DMF (50 mls) over a 1.5 h period. The reaction was allowed to stir for 6 h at ambient temperature. The reaction mixture was then poured into a beaker containing ice and water (800 mls). This mixture was extracted with a 1:1 mixture of ether:petroleum ether (6×200 mls) and the combined organics were washed with water (300 mls) and brine (1×200 mL), dried over sodium sulfate, filtered and concentrated in vacuo to yield 15.7 g of crude product 41 which was chromatographed on a silica gel column with 15% CHCl₃ in hexane to afford 10.47 g (64%) of [2-(4-Bromo-2-ethyl-5-methoxy-phenyl)-ethoxy]-tert-butyl-dimethyl-silane 41.

¹H NMR (CDCl₃): 0.001 (s-6H), 0.86 (s-9H), 1.17 (t-3H), 2.57 (q-2H), 2.79 (t-2H), 3.76 (t-2H), 3.84 (s-3H), 6.72 (s-1H), 7.30 (s-1H).

### Preparation 32

### [2-(4-Boronic acid-2-ethyl-5-methoxy-phenyl)-ethoxy]-tert-butyl-dimethyl-silane (42)

Under a N₂ atmosphere in 100 mL of anhydrous THF was added 9.70 g (0.026 mol) [2-(4-Bromo-2-ethyl-5-methoxy-phenyl)-ethoxy]-tert-butyl-dimethyl-silane. The solution was cooled to −78° C. and 11.43 mL (0.029 mol) of a 2.5 M solution of butyl lithium was added dropwise while maintaining the temperature below −70° C. The reaction mixture was stirred at −78° C. for 1 hr at which point 4.93 mL (0.029 mol) of triethyl borate was added. The reaction mixture was stirred at −78° C. for 2 hr and was then allowed to warm to 23° C. over a 2 hr period and was quenched with 100 mL of saturated NH₄Cl, the pH was adjusted to 7.0 with 1M HCl and the resultant solution was extracted with ethyl acetate (2×200 mL). The combined extracts were washed with brine (1×100 mL), dried over sodium sulfate, filtered and concentrated in vacuo to afford 10.2 g (100%) of crude [2-(4-Boronic acid-2-ethyl-5-methoxy-phenyl)-ethoxy]-tert-butyl-dimethyl-silane 42 which was used directly in the next reaction.

### Preparation 33

### N-(6-{4-[2-(tert-Butyl-dimethyl-silanyloxy)-ethyl]-5-ethyl-2-methoxy-phenyl}-pyridin-2-yl)-2,2-dimethyl-propionamide (43)

Under a nitrogen atmosphere was combined 6.07 g (0.024 mol) of 2-bromo-6-(N-2,2-dimethylpropamido)pyridine, 8.79 g (0.026 mol) of crude [2-(4-Boronic acid-2-ethyl-5-methoxy-phenyl)-ethoxy]-tert-butyl-dimethyl-silane, 10.7 g (0.096 mol) of sodium carbonate and 5.56 g (0.0048 mol) of tetrakis(triphenylphosphine)palladium(0) in 270 mL of ethanol and 30 mL of water. The solution was heated to 90° C. for 18 hrs and then cooled at which point the reaction mixture was concentrated in vacuo. The resultant residue was partitioned between ethyl acetate (600 mL) and water (600 mL). The aqueous layer was extracted again with ethyl acetate (600 mL) and the combined organic extracts were washed with brine (1×200 mL), dried over sodium sulfate, filtered and concentrated in vacuo to yield crude product which was chromatographed on a silica gel column with 20% ethyl ether in hexane to afford 6.0 g (53.1%) of N-(6-{4-[2-(tert-Butyl-dimethyl-silanyloxy)-ethyl]-5-ethyl-2-methoxy-phenyl}-pyridin-2-yl)-2,2-dimethyl-propionamide 43.

¹H NMR (CDCl₃): 0.003 (s-6H), 0.89 (s-9H), 1.22 (t-3H), 1.32 (s-9H), 2.67 (q-2H), 2.88 (t-2H), 3.76-3.80 (m-5H), 6.81 (s-1H), 7.46-7.50 (m-2H), 7.68 (t-1H), 8.05 (bs-1H), 8.14 (d-1H).

### Preparation 34

### N-{6-[5-Ethyl-4-(2-hydroxy-ethyl)-2-methoxy-phenyl]-pyridin-2-yl)-2,2-dimethyl-propionamide (44)

Under a nitrogen atmosphere in a THF (100 mL) solution containing 6.00 g (0.013 mol) of N-(6-{4-[2-(tert-Butyl-dimethyl-silanyloxy)-ethyl]-5-ethyl-2-methoxy-phenyl}-pyridin-2-yl)-2,2-dimethyl-propionamide was added 32 mL (0.032 mol) of 1 M TBAF in THF. The solution was allowed to stir at ambient temperature for 16 hours at which point the reaction mixture was concentrated in vacuo. The resultant residue was partitioned between ethyl acetate (400 mL) and water (300 mL). The aqueous layer was extracted again with ethyl acetate (200 mL) and the combined organic extracts were washed with water (1×200 mL) and brine (1×200 mL), dried over sodium sulfate, filtered and concentrated in vacuo to yield 6.2 g of crude 31 which was chromatographed on a silica gel column with 50% ethyl acetate in hexane to afford 4.28 g (92.3%) of N-{6-[5-Ethyl-4-(2-hydroxy-ethyl)-2-methoxy-phenyl]-pyridin-2-yl}-2,2-dimethyl-propionamide 44.

¹H NMR (CDCl₃): 1.23 (t-3H), 1.32 (s-9H), 1.44 (t-1H), 2.67 (q-2H), 2.94 (t-2H), 3.81 (s-3H), 3.85-3.87 (m-2H), 6.81 (s-1H), 7.47-7.49 (m-2H), 7.69 (t-1H), 8.05 (bs-1H), 8.15 (d-1H).

### Preparation 35

### N-{6-[5-Ethyl-2-methoxy-4-(2-oxo-ethyl)-phenyl]-pyridin-2-yl}-2,2-dimethyl-propionamide (45)

Under a nitrogen atmosphere in a methylene chloride (150 mL) solution containing 1.00 g (0.0028 mmol) of N-{6-[5-Ethyl-4-(2-hydroxy-ethyl)-2-methoxy-phenyl]-pyridin-2-yl}-2,2-dimethyl-propionamide was added 1.78 g (0.0042 mmol) of Dess-Martin periodinate. The solution was allowed to stir for 1 h at which point the reaction mixture was diluted with 25 ml of ether and filtered. The resultant organic solution was washed with water (20 mL). The aqueous layer was extracted again with ether (2×100 mL) and the combined organic extracts were washed with 20% sodium thiosulfate, saturated sodium bicarbonate and brine and were dried over sodium sulfate, filtered and concentrated in vacuo to yield 1.10 g of crude product which was chromatographed on a silica gel column with 20% ethyl acetate in hexane to afford 779 mg (78.5%) of N-{6-[5-Ethyl-2-methoxy-4-(2-oxo-ethyl)-phenyl]-pyridin-2-yl}-2,2-dimethyl-propionamide 45.

¹H NMR (CDCl₃): 1.22 (t-3H), 1.32 (s-9H), 2.60 (q-2H), 3.73 (s-2H), 3.81 (s-3H), 6.76 (s-1H), 7.49 (dd-1H), 7.54 (s-1H), 7.70 (t-1H), 8.10 (bs-1H), 8.17 (dd-1H), 9.71 (s-1H).

### Preparation 36

### N-}6-[4-(2-Dimethylamino-ethyl)-5-ethyl-2-methoxy-phenyl]-pyridin-2-yl}-2,2-dim propionamide (46)

Under a nitrogen atmosphere in dichloroethane (10 mL) was combined 87.4 mg (0.246 mmol) of N-{6-[5-Ethyl-2-methoxy-4-(2-oxo-ethyl)-phenyl]-pyridin-2-yl}-2,2-dimethyl-propionamide, 40 mg (0.493 mmol) of N,N-dimethylamine hydrochloride, 100 mg (0.468 mmol) of sodium triacetoxyborohydride and 30 mg (0.493 mmol) of acetic acid. The solution was allowed to stir for 16 hrs at 23° C. which point the reaction mixture was washed with 1M NaOH (30 mL) and then the aqueous layer was extracted again with dichloromethane (2×50 mL) and the combined organic extracts were dried over sodium sulfate, filtered and concentrated in vacuo to yield 87 mg of crude product which was chromatographed on a silica gel column with 50% ethyl acetate in hexane to afford 41 mg (43.5%) of N-{6-[4-(2-Dimethylamino-ethyl)-5-ethyl-2-methoxy-phenyl]-pyridin-2-yl}-2,2-dimethyl-propionamide 46.

¹H NMR (CDCl₃): 1.23 (t-3H), 1.32 (s-9H), 2.38 (bs-6H), 2.55 (m-2H), 2.65 (q-2H), 2.86 (m-2H), 3.81 (s-3H), 6.78 (s-1H), 7.47-7.50 (m-2H), 7.68 (t-1H), 8.05 (bs-1H), 8.14 (dd-1H).

### EXAMPLE 8

### 6-[4-(2-Dimethylamino-ethyl)-5-ethyl-2-methoxy-phenyl]-pyridin-2-ylamine (47)

Under a N₂ atmosphere in 5 mL of dioxane was combined 41 mg (0.107 mmol) of N-{6-[4-(2-Dimethylamino-ethyl)-5-ethyl-2-methoxy-phenyl]-pyridin-2-yl}-2,2-dimethyl-propionamide and 4 mL of 1 N NaOH. The reaction was allowed to reflux with stirring for 6 days and was allowed to cool to ambient temperature and was partitioned between dichloromethane and brine. The aqueous layer was extracted a with dichloromethane (2×50 mL) and the combined organic extracts were dried over sodium sulfate, filtered and concentrated in vacuo to yield crude product which was chromatographed on a silica gel column with 10% methanol in dichloromethane to afford 18 mg (58.6%) of 6-[4-(2-Dimethylamino-ethyl)-5-ethyl-2-methoxy-phenyl]-pyridin-2-ylamine 47.

¹H NMR (CDCl₃): 1.21 (t-3H), 2.33 (s-6H), 2.46-2.50 (m-2H), 2.64 (q-2H), 2.81-2.83 (m-2H), 3.80 (s-3H), 4.41 (bs-2H), 6.41 (dd-1H), 6.74 (s-1H), 7.11 (dd-1H), 7.41-7.48 (m-2H).

The following compounds were synthesized according to the same procedure used for the conversion of (45) to (47) described above:

| | |
|---|---|
| Example 8(a) | 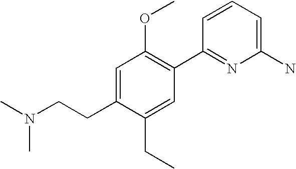 |
| Example 8(b) | 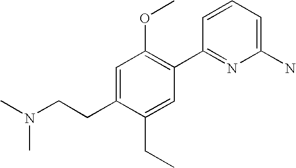 |
| Example 8(c) | 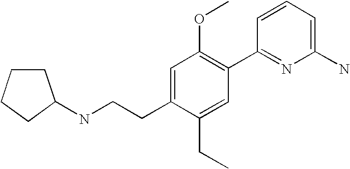 |
| Example 8(d) | 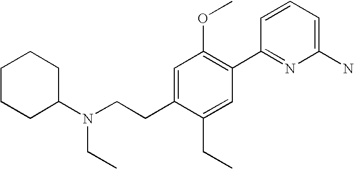 |
| Example 8(e) | 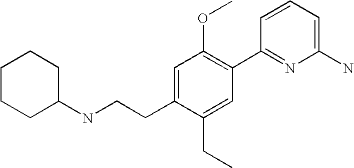 |
| Example 8(f) | 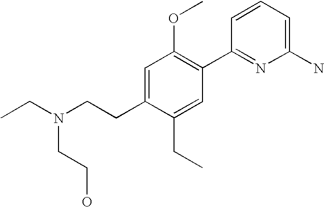 |
| Example 8(g) | 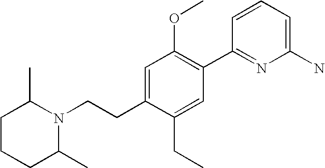 |
| Example 8(h) | 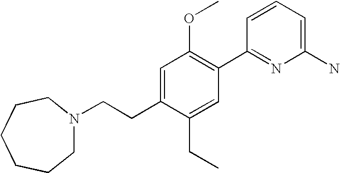 |
| Example 8(i) | 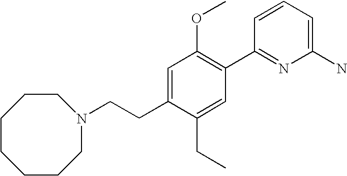 |
| Example 8(j) | 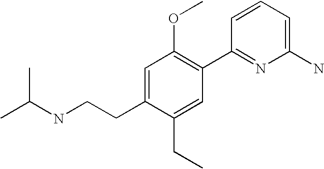 |
| Example 8(k) | 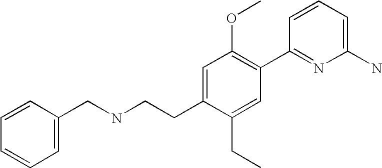 |
| Example 8(l) | 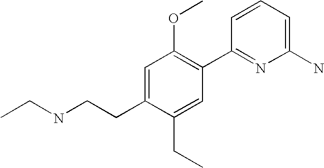 |
| Example 8(m) | 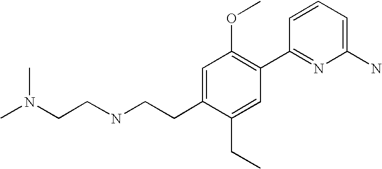 |
| Example 8(n) | 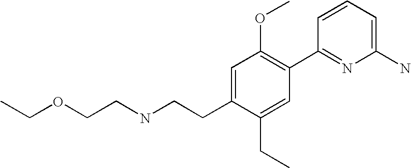 |
| Example 8(o) | 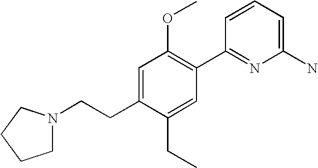 |
| Example 8(p) | 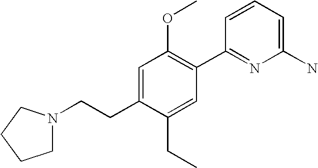 |
| Example 8(q) | 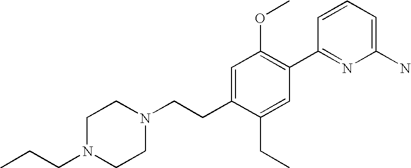 |
| Example 8(r) | 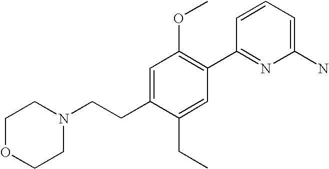 |
| Example 8(s) | 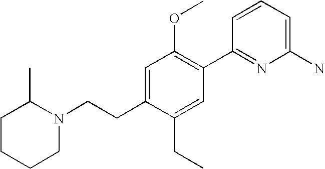 |
| Example 8(t) | 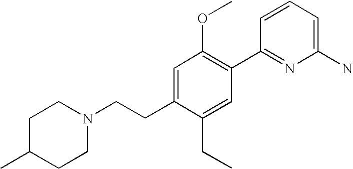 |
| Example 8(u) | 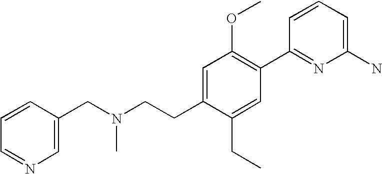 |
| Example 8(v) | 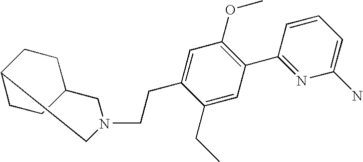 |
| Example 8(w) | 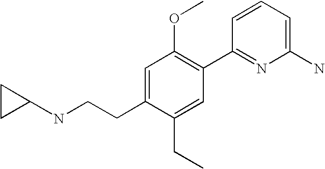 |
| Example 8(x) | 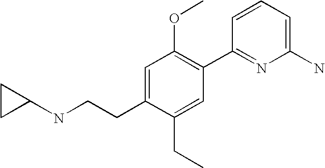 |
| Example 8(y) | 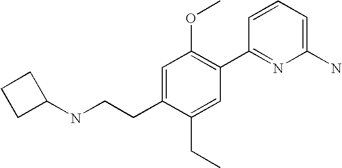 |
| Example 8(z) | 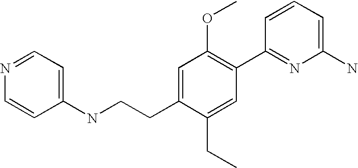 |
| Example 8(aa) | 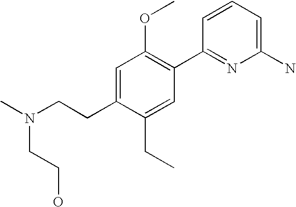 |
| Example 8(bb) | 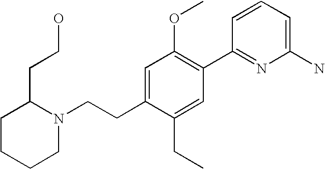 |
| Example 8(cc) | 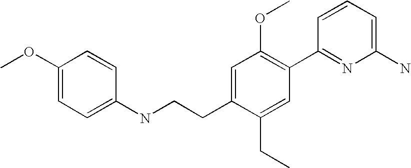 |
| Example 8(dd) | 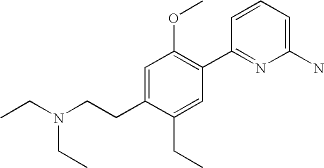 |
| Example 8(ee) | 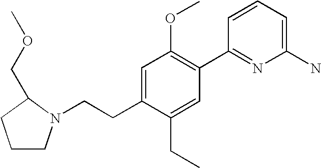 |
| Example 8(ff) | 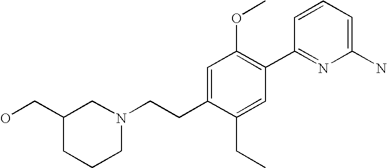 |
| Example 8(gg) | 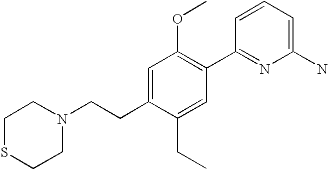 |
| Example 8(hh) | 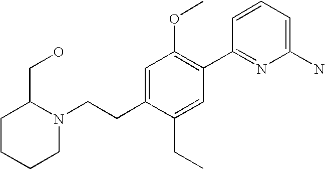 |
| Example 8(ii) | 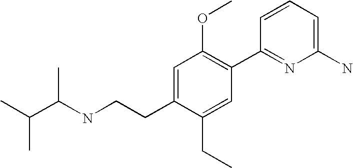 |
| Example 8(jj) | 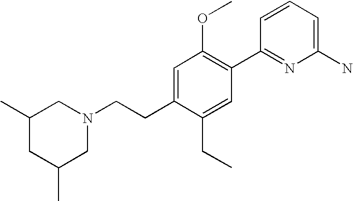 |
| Example 8(kk) | 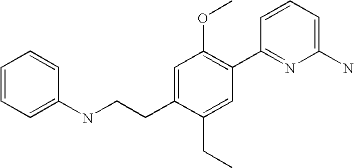 |
| Example 8(ll) | 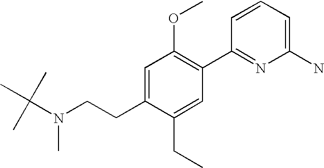 |
| Example 8(mm) | 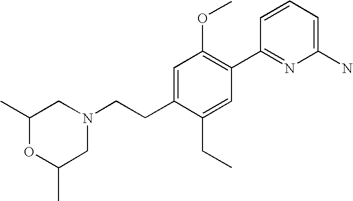 |
| Example 8(nn) | 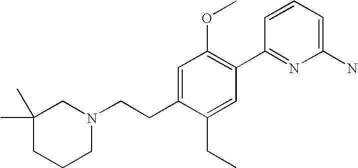 |
| Example 8(oo) | 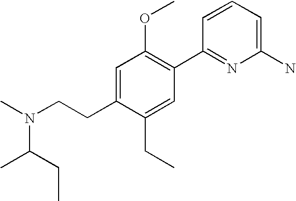 |
| Example 8(pp) | 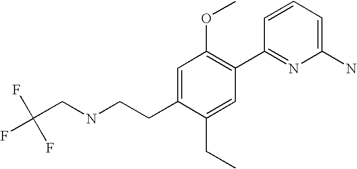 |
| Example 8(qq) | 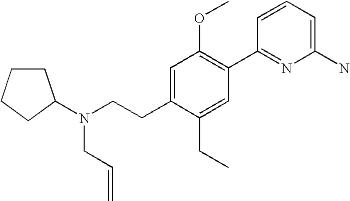 |
| Example 8(rr) | 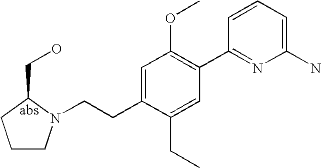 |
| Example 8(ss) | 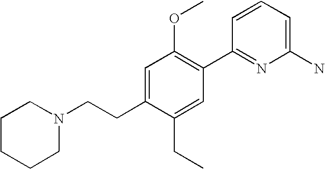 |
| Example 8(tt) | 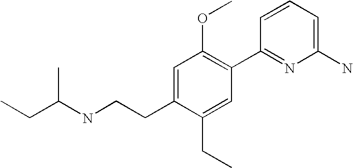 |
| Example 8(uu) | 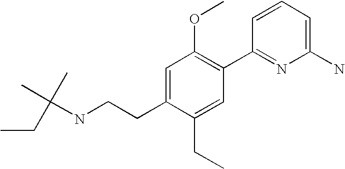 |
| Example 8(vv) | 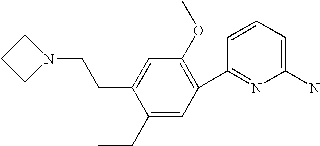 |
| Example 8(ww) | 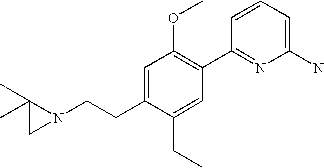 |
| Example 8(xx) | 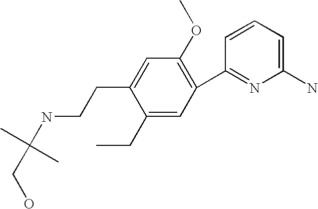 |

### Preparation 37

### [2-(2,5-dimethoxy-phenyl)-ethoxy]-dimethyl-silane (48)

6.15 g (0.0338 mol) of 2-(2,5-Dimethoxy-phenyl)-ethanol was dissolved in 60 mL of anhydrous THF under a N₂ atmosphere. 4.60 g (0.0675 mol) of imidazole was added followed by 5.60 g (0.0371 mol) of t-butyl dimethylsilylchloride. The reaction mixture was allowed to stir at ambient temperature for 2 hours. Ethyl acetate was added and the reaction was diluted with water (200 mL). The aqueous layer was separated and extracted with ethyl acetate (2×300 mL) and the combined organic extracts were dried over sodium sulfate, filtered and concentrated in vacuo to afford crude product which was loaded onto a silica gel column. Using 0-25% ethyl acetate in hexane yielded 8.32 g (82%) of tert-Butyl-[2-(2,5-dimethoxy-phenyl)-ethoxy]-dimethyl-silane 48.

¹H NMR (CDCl₃): 0.001 (s-6H), 0.86 (s-9H), 2.81 (t-2H, J=7.24 Hz), 3.73-3.78 (m-8H), 6.69-6.76 (m-3H).

### Preparation 38

### [2-(4-Bromo-2,5-dimethoxy-phenyl)-ethoxy]-tert-butyl-dimethyl-silane (49)

Under a N₂ atmosphere in 100 mL of carbon tetrachloride was combined 8.19 g (0.0276 mol) of tert-butyl-[2-(2,5-dimethoxy-phenyl)-ethoxy]-dimethyl-silane and 5.16 g (0.0290 mmol) of NBS followed by 25 g of silica gel 60 (EM Science). The reaction was allowed to stir for 16 h in the absence of light. The reaction mixture was filtered and the silica gel was washed with dichloromethane The filtrate was washed with with dichloromethane (1×300 mL) and the combined organic extracts were washed with 1 M NaOH (2×300 mL), dilute NaHSO₃ (1×200 mL) and brine (1×200 mL), dried over magnesium sulfate, filtered and concentrated in vacuo to yield crude product 49 which was chromatographed on a silica gel column with 0-40% CH₂Cl₂ in hexane to afford 3.15 g (30%) of tert-butyl-dimethyl-(4-bromo-2-ethyl-5-methoxy-benzyloxy)-silane 49. Washing the column with 5% MeOH in methylene chloride afforded 3.04 g of 2-(4-Bromo-2,5-dimethoxy-phenyl)-ethanol.

¹H NMR (CDCl₃): 0.001 (s-6H), 0.85 (s-9H), 2.78 (t-2H), 3.73-3.78 (m-5H), 3.81 (s-3H), 6.77 (s-1H), 6.99 (s-1H).

### Preparation 39

### [2-(4-Boronic acid-2,5-dimethoxy-phenyl)-ethoxy]-tert-butyl-dimethyl-silane (50)

Under a N₂ atmosphere in 20 mL of anhydrous THF was added 3.14 g (0.00837 mol) of tert-butyl-dimethyl-(4-bromo-2-ethyl-5-methoxy-benzyloxy)-silane. The solution was cooled to −78° C. and 4.66 mL (0.01165 mol) of a 2.5 M solution of butyl lithium was added dropwise while maintaining the temperature below −70° C. The reaction mixture was stirred at −78° C. for 1 hr at which point 1.98 mL (0.01165 mol) of triethyl borate was added. The reaction mixture was stirred at −78° C. for 2 hr and was then allowed to warm to 23° C. over a 2 hr period and was quenched with 100 mL of saturated NH₄Cl, the pH was adjusted to 5.0 with 1M HCl and the resultant solution was extracted with ethyl acetate (2×200 mL). The combined extracts were washed with brine (1×100 mL), dried over sodium sulfate, filtered and concentrated in vacuo to afford 3.04 g (100%) of crude [2-(4-Boronic acid-2,5-dimethoxy-phenyl)-ethoxy]-tert-butyl-dimethyl-silane 50 which was used directly in the next reaction.

¹H NMR (CDCl₃): 0.001 (s-6H), 0.85 (s-9H), 2.84 (t-2H), 3.73-3.78 (m-5H), 3.85 (s-3H), 5.87 (s-2H), 6.77 (s-1H), 7.24 (s-1H).

### Preparation 40

### N-(6-{4-[2-(tert-Butyl-dimethyl-silanyloxy)-ethyl]-2,5-dimethoxy-phenyl}-pyridin-2-yl)-2,2-dimethyl-propionamide (12)

Under a nitrogen atmosphere was combined 2.09 g (0.00812 mol) of 2-bromo-6-(N-2,2-dimethylpropamido)pyridine, 3.04 g (0.00893 mol) of crude [2-(4-Boronic acid-2,5-dimethoxy-phenyl)-ethoxy]-tert-butyl-dimethyl-silane, 3.45 g (0.0325 mol) of sodium carbonate and 210 mg of tetrakis(triphenylphosphine)palladium(0) in 45 mL of ethanol and 5 mL of water. The solution was allowed to reflux for 18 hrs at which point the reaction mixture was concentrated in vacuo. The resultant yellow residue was partitioned between ethyl acetate (200 mL) and water (200 mL). The aqueous layer was extracted again with ethyl acetate (200 mL) and the combined organic extracts were washed with brine (1×200 mL), dried over sodium sulfate, filtered and concentrated in vacuo to yield crude product which was chromatographed on a silica gel column with 5-10% ethyl acetate in hexane to afford 2.97 g of N-(6-{4-[2-(tert-Butyl-dimethyl-silanyloxy)-ethyl]-2,5-dimethoxy-phenyl}-pyridin-2-yl)-2,2-dimethyl-propionamide 51.

¹H NMR (CDCl₃): 0.012 (s-6H), 0.88 (s-9H), 1.33 (s-9H), 2.87 (t-2H, J=7.06 Hz),3.77 (s-3H), 3.73-3.78 (m-2H), 3.83 (s-3H), 6.85 (s-1H), 7.19 (s-1H), 7.52 (dd-1H), 7.69 (t-1H), 8.05 (bs-1H), 8.14 (dd-1H).

### Preparation 41

### N-{6-[4-(2-Hydroxy-ethyl)-2,5-dimethoxy-phenyl]-pyridin-2-yl}-2,2-dimethyl-propionamide (52)

Under a nitrogen atmosphere in a THF (30 mL) solution containing 2.96 (00626 mol) g of N-(6-{4-[2-(tert-Butyl-dimethyl-silanyloxy)-ethyl]-2,5-dimethoxy-phenyl}-pyridin-2-yl)-2,2-dimethyl-propionamide 51 was added 10 mL (0.010 mol) of 1M TBAF in THF. The solution was allowed to stir for 90 min at which point the reaction mixture was concentrated in vacuo. The resultant residue was partitioned between ethyl acetate (200 mL) and water (200 mL). The aqueous layer was extracted again with ethyl acetate (200 mL) and the combined organic extracts were washed with water (1×100 mL) and brine (1×100 mL), dried over sodium sulfate, filtered and concentrated in vacuo to yield 3.26 g of crude N-{6-[4-(2-Hydroxy-ethyl)-2,5-dimethoxy-phenyl]-pyridin-2-yl}-2,2-dimethyl-propionamide 52 which was triturated with hexane to afford 2.20 g (98%) of 52 as a white semisolid which was washed with hexane and dried

¹H NMR (CDCl₃): 1.32 (s-9H), 2.92 (t-2H), 3.77 (s-3H), 3.80-3.87 (m-2H), 3.84 (s-3H), 6.83 (s-1H), 7.21 (s-1H), 7.51 (dd-1H), 7.70 (t-1H), 8.06 (bs-1H), 8.16 (dd-1H).

### Preparation 42

### N-{6-[2,5-Dimethoxy-4-(2-oxo-ethyl)-phenyl]-pyridin-2-yl}-2,2-dimethyl-propionamide (53)

Under a nitrogen atmosphere in a methylene chloride (3 mL) solution containing 50 mg (0.139 mmol) of N-{6-[4-(2-Hydroxy-ethyl)-2,5-dimethoxy-phenyl]-pyridin-2-yl}-2,2-dimethyl-propionamide was added 236 mg (0.556 mmol) of Dess-Martin periodinate. The solution was allowed to stir for 1 h at which point the reaction mixture was diluted with 16 ml of ether and filtered. The resultant organic solution was washed with water (20 mL). The aqueous layer was extracted again with ether (1×20 mL) and the combined organic extracts were washed with 20% sodium thiosulfate, saturated sodium bicarbonate and brine and were dried over sodium sulfate, filtered and concentrated in vacuo to yield crude product which was chromatographed on a silica gel column with 40% ethyl acetate in hexane to afford 27 mg (54%) of N-{6-[2,5-Dimethoxy-4-(2-oxo-ethyl)-phenyl]-pyridin-2-yl}-2,2-dimethyl-propionamide 53.

¹H NMR (CDCl₃): 1.32 (s-9H), 3.67 (s-2H), 3.78 (s-3H), 3.85 (s-3H), 6.80 (s-1H), 7.26 (s-1H), 7.52 (dd-1H), 7.71 (t-1H), 8.04 (bs-1H), 8.18 (dd-1H), 9.68 (s-1H).

### Preparation 43

### N-{6-[4-(2-Dimethylamino-ethyl)-2,5-dimethoxy-phenyl]-pyridin-2-yl}-2,2-dimethyl-propionamide (54)

Under a nitrogen atmosphere in dichloroethane (3 mL) was combined 27 mg (0.076 mmol) of N-{6-[2,5-dimethoxy-4-(2-oxo-ethyl)-phenyl]-pyridin-2-yl}-2,2-dimethyl-propionamide, 42 ul (0.084 mmol) of 2M N,N-dimethylamine in THF, 31 mg (0.144 mmol) of sodium triacetoxyborohydride and 9 uL (0.144 mmol) of acetic acid. The solution was allowed to stir for 16 hours at 23° C. at which point the reaction mixture was washed with 1 M NaOH and then the aqueous layer was extracted again with dichloromethane (2×40 mL) and the combined organic extracts were dried over sodium sulfate, filtered and concentrated in vacuo to yield crude product which was chromatographed on a silica gel column with 5% methanol in dichloromethane to afford 12 mg (41%) of N-{6-[4-(2-Dimethylamino-ethyl)-2,5-dimethoxy-phenyl]-pyridin-2-yl}-2,2-dimethyl-propionamide 54.

¹H NMR (CDCl₃): 1.31 (s-9H), 2.31 (s-6H), 2.51 (m-2H), 2.81 (m-2H), 3.77 (s-3H), 3.83 (s-3H), 6.81 (s-1H), 7.18 (s-1H), 7.48-7.52 (dd-1H), 7.69 (t-1H), 8.05 (bs-1H), 8.14 (dd-1H).

### EXAMPLE 9

### 6-[4-(2-Dimethylamino-ethyl)-2,5-dimethoxy-phenyl]-pyridin-2-ylamine (55)

Under a N₂ atmosphere in 1.5 uL of dioxane was combined 12 mg (0.031 mmol) of N-{6-[4-(2-Dimethylamino-ethyl)-2,5-dimethoxy-phenyl]-pyridin-2-yl}-2,2-dimethyl-propionamide and 3 mL of 1N NaOH. The reaction was allowed to reflux with stirring for 18 hours. The resultant solution was extracted a with dichloromethane (3×50 mL) and the combined organic extracts were dried over sodium sulfate, filtered and concentrated in vacuo to yield crude product which was chromatographed on a silica gel column with 5-15% methanol in dichloromethane to afford 9 mg (100%) of 6-[4-(2-dimethylamino-ethyl)-2,5-dimethoxy-phenyl]-pyridin-2-ylamine 55.

¹H NMR (CDCl₃): 2.32 (s-6H), 2.53 (m-2H), 2.81 (m-2H), 3.77 (s-3H), 3.83 (s-3H), 4.46 (bs-2H), 6.42 (dd-1H), 6.79 (s-1H), 7.16 (d-1H), 7.23 (s-1H), 7.45 (t-1H).

1. A compound of formula VI 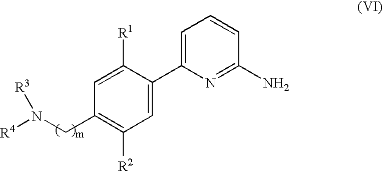
wherein R¹ is selected from methyl, ethyl, propyl, butyl, isopropyl, 2-methylpropyl, t-butyl, methoxy, ethoxy, and propoxy;
R² is selected from hydrogen, methyl, ethyl, propyl, butyl, isopropyl, 1-methylpropyl, 2-methylpropyl, t-butyl, methoxy, ethoxy, and propoxy;
m is one, two or three;
R³ and R⁴ are selected, independently, from R⁷; phenyl; 5 or 6 membered heteroaryl containing from 1 to 4 heteroatoms independently selected from O, N, and S; and straight chain or branched (C₁-C₆) alkyl substituted with from 1 to 3 substituents selected independently from R⁶, —CF₃, halo, (i.e. bromine, chlorine, iodine, and fluorine), —NR⁷R⁸, (C₃-C₆) cycloalkyl, 3 to 9 membered heterocycloalkyl containing 1 or 2 heteroatoms independently selected from O, N, and S, phenyl, and 5 or 6 membered heteroaryl containing from 1 to 4 heteroatoms independently selected from O, N, and S;
wherein said phenyl, heteroaryl, cycloalkyl, and heterocycloalkyl groups of R³ and R⁴ are optionally independently substituted with from 1 to 3 substituents independently selected from R⁶ and straight chain or branched C₁-C₆ alkyl optionally comprising 1 or 2 double or triple bonds;
or R³ and R⁴ are connected, with the nitrogen atom to which they are attached, to form a 3 to 9 membered heterocyclic ring, which heterocyclic optionally comprises from one to three heteroatoms in addition to said nitrogen atom, which optional heteroatoms are selected independently from O, S, and N;
wherein said heterocyclic ring formed by R³ and R⁴ optionally is fused to form a fused ring system with one or two aromatic rings selected independently from benzene rings and heteroaromatic rings, which aromatic rings share two carbon atoms with said heterocyclic ring; or which heterocyclic ring formed by R³ and R⁴ is optionally fused to form a fused or spiro ring system to a 3 to 8 membered carbocyclic ring which shares one or two carbon atoms with said heterocyclic ring; wherein fused or spiro ring systems contain up to 15 ring members;
and wherein said heterocyclic ring, said optional aromatic rings, and said optional carbocyclic ring, are each optionally and independently substituted with from 1 to 3 substituents independently selected from R⁶, —O—(C₁-C₆ alkyl)-R⁶, —S—(C₁-C₆ alkyl)-R⁶, straight chain or branched (C₁-C₆) alkyl optionally substituted with R⁶, —C(═O)O—((C₀-C₆) alkyl), 3 to 6 membered cycloalkyl, phenyl, benzyl, and 5 or 6 membered heteroaryl; wherein said cycloalkyl, phenyl, benzyl, and heteroaryl are independently optionally substituted with from 1 to 3 substituents independently selected from R⁵;
R⁵ is selected from R⁶, straight chain or branched (C₁-C₆ alkyl), —(C₁-C₆ alkyl)-R⁶, and 5 or 6 membered heteroaryl optionally substituted with 1 or 2 substituents independently selected from R⁶, —NR⁷R⁸, straight chain or branched (C₁-C₆) alkyl, and (C₁-C₆) alkyl-R⁶;
R⁶ is selected from —O—R⁷ and —S—R⁷;
R⁷ is selected from H and straight chain or branched (C₁-C₆) alkyl (e.g. methyl, ethyl, propyl, butyl, isopropyl, 1-methylpropyl, 2-methylpropyl, t-butyl, pentyl, 3-methylbutyl, 1,2-dimethylpropyl, or 1,1-dimethylbutyl) optionally comprising 1 or 2 double or triple bonds; and
R⁸ is selected from H and straight chain or branched (C₁-C₆) alkyl;
or a pharmaceutically acceptable salt thereof.

2. A compound according to claim 1, or a pharmaceutically acceptable salt thereof, wherein m is 1 or 2.

3. A compound according to claim 1, or a pharmaceutically acceptable salt thereof, wherein m is 3.

4. A compound according to claim 1, or a pharmaceutically acceptable salt thereof, wherein R³ and R⁴ are selected from H and methyl.

5. A compound according to claim 4, or a pharmaceutically acceptable salt thereof, wherein, R³ and R⁴ are both methyl.

6. A compound according to claim 4, or a pharmaceutically acceptable salt thereof, wherein one of R³ and R⁴ is methyl, and the other of R³ and R⁴ is H.

7. A compound according to claim 1, or a pharmaceutically acceptable salt thereof, wherein R¹ is selected from methyl, ethyl, and methoxy, and R² is selected from ethyl and methoxy.

8. A compound according to claim 7, or a pharmaceutically acceptable salt thereof, wherein R¹ is methoxy.

9. A compound according to claim 8, or a pharmaceutically acceptable salt thereof, wherein R¹ and R² are both methoxy.

10. A compound according to claim 8, or a pharmaceutically acceptable salt thereof, wherein R² is ethyl.

11. A compound according to claim 1, or a pharmaceutically acceptable salt thereof, with the proviso that when R¹ is —OCH₃, R² is ethyl, and R⁴ is methyl, then R³ is not hydrogen or methyl.

12. A pharmaceutical composition for treating a condition selected from the group consisting of migraine inflammatory diseases, stroke, acute, chronic and neuropathic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions, emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, ovulation, dilated cardiomyopathy, acute spinal cord injury, Huntington's disease, Parkinson's disease, glaucoma, macular degeneration, diabetic neuropathy, diabetic nephropathy and cancer in a mammal, comprising an amount of a compound according to claim 1 that is effective in treating such condition and a pharmaceutically acceptable carrier.

13. A method of treating a condition selected from the group consisting of migraine inflammatory diseases, stroke, acute, chronic and neuropathic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions, emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, ovulation, dilated cardiomyopathy, acute spinal cord injury, Huntington's disease, Parkinson's disease, glaucoma, macular degeneration, diabetic neuropathy, diabetic nephropathy and cancer in a mammal, comprising administering to said mammal an amount of a compound according to claim 1 that is effective in treating such condition.

14. A pharmaceutical composition for inhibiting nitric oxide synthase (NOS) in a mammal, comprising a NOS inhibiting effective amount of a compound according to claim 1 and a pharmaceutically acceptable carrier.

15. A method of inhibiting NOS in a mammal, comprising administering to said mammal a NOS inhibiting effective amount of a compound according to claim 1.

16. A pharmaceutical composition for treating a condition selected from the group consisting of migraine, inflammatory diseases, stroke, acute, chronic and neuropathic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions, emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, ovulation, dilated cardiomyopathy, acute spinal cord injury, Huntington's disease, Parkinson's disease, glaucoma, macular degeneration, diabetic neuropathy, diabetic nephropathy and cancer in a mammal, comprising a NOS inhibiting effective amount of a compound according to claim 1 and a pharmaceutically acceptable carrier.

17. A method of treating a condition selected from the group consisting of migraine, inflammatory diseases, stroke, acute, chronic and neuropathic pain, hypovolemic shock, traumatic shock, reperfusion injury, Crohn's disease, ulcerative colitis, septic shock, multiple sclerosis, AIDS associated dementia, neurodegenerative diseases, neuron toxicity, Alzheimer's disease, chemical dependencies and addictions, emesis, epilepsy, anxiety, psychosis, head trauma, adult respiratory distress syndrome (ARDS), morphine induced tolerance and withdrawal symptoms, inflammatory bowel disease, osteoarthritis, rheumatoid arthritis, ovulation, dilated cardiomyopathy, acute spinal cord injury, Huntington's disease, Parkinson's disease, glaucoma, macular degeneration, diabetic neuropathy, diabetic nephropathy and cancer in a mammal, comprising administering to said mammal a NOS inhibiting effective amount of a compound according to claim 1.

18. A method of inhibiting neurological damage caused by impairment of glucose and/or oxygen to the brain in a mammal, which method comprises administering to the mammal an amount of a NOS inhibitor, which amount is effective in inhibiting neurological damage.

19. A method according to claim 18, wherein the NOS inhibitor is selected from the group consisting of:
(a) a compound of formula I 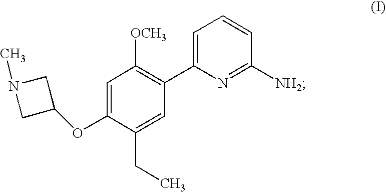
(b) a compound of formula II 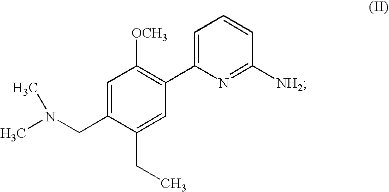
(c) a compound of formula III 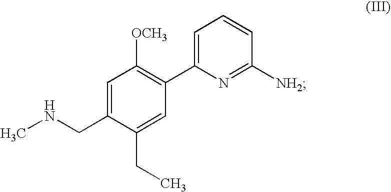
(d) a compound of formula IV 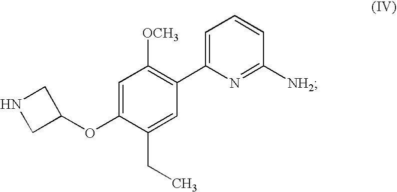
(e) a compound of formula V 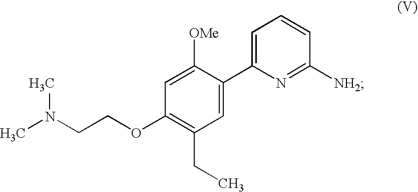
(f) a compound of formula VI 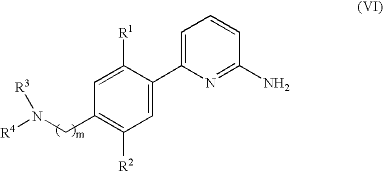
wherein R¹ is selected from methyl, ethyl, propyl, butyl, isopropyl, 2-methylpropyl, t-butyl, methoxy, ethoxy, and propoxy;
R² is selected from hydrogen, methyl, ethyl, propyl, butyl, isopropyl, 1-methylpropyl, 2-methylpropyl, t-butyl, methoxy, ethoxy, and propoxy;
m is one, two or three;
R³ and R⁴ are selected, independently, from R⁷; phenyl; 5 or 6 membered heteroaryl containing from 1 to 4 heteroatoms independently selected from O, N, and S; and straight chain or branched (C₁-C₆) alkyl substituted with from 1 to 3 substituents selected independently from R⁶, —CF₃, halo, (i.e. bromine, chlorine, iodine, and fluorine), —NR⁷R⁸, (C₃-C₆) cycloalkyl, 3 to 9 membered heterocycloalkyl containing 1 or 2 heteroatoms independently selected from O, N, and S, phenyl, and 5 or 6 membered heteroaryl containing from 1 to 4 heteroatoms independently selected from O, N, and S;
wherein said phenyl, heteroaryl, cycloalkyl, and heterocycloalkyl groups of R³ and R⁴ are optionally independently substituted with from 1 to 3 substituents independently selected from R⁶ and straight chain or branched C₁-C₆ alkyl optionally comprising 1 or 2 double or triple bonds;
or R³ and R⁴ are connected, with the nitrogen atom to which they are attached, to form a 3 to 9 membered heterocyclic ring, which heterocyclic optionally comprises from one to three heteroatoms in addition to said nitrogen atom, which optional heteroatoms are selected independently from O, S, and N;
wherein said heterocyclic ring formed by R³ and R⁴ optionally is fused to form a fused ring system with one or two aromatic rings selected independently from benzene rings and heteroaromatic rings, which aromatic rings share two carbon atoms with said heterocyclic ring; or which heterocyclic ring formed by R³ and R⁴ is optionally fused to form a fused or spiro ring system to a 3 to 8 membered carbocyclic ring which shares one or two carbon atoms with said heterocyclic ring; wherein fused or spiro ring systems contain up to 15 ring members;
and wherein said heterocyclic ring, said optional aromatic rings, and said optional carbocyclic ring, are each optionally and independently substituted with from 1 to 3 substituents independently selected from R⁶, —O—(C₁-C₆ alkyl)-R⁶, —S—(C₁-C₆ alkyl)-R⁶, straight chain or branched (C₁-C₆) alkyl optionally substituted with R⁶, —C(═O)O—((C₁-C₆) alkyl), 3 to 6 membered cycloalkyl, phenyl, benzyl, and 5 or 6 membered heteroaryl; wherein said cycloalkyl, phenyl, benzyl, and heteroaryl are independently optionally substituted with from 1 to 3 substituents independently selected from R⁵;
R⁵ is selected from R⁶, straight chain or branched (C₁-C₆ alkyl), —(C₁-C₆ alkyl)-R⁶, and 5 or 6 membered heteroaryl optionally substituted with 1 or 2 substituents independently selected from R⁶, —NR⁷R⁸, straight chain or branched (C₁-C₆) alkyl, and (C₁-C₆) alkyl-R⁶;
R⁶ is selected from —O—R⁷ and —S—R⁷;
R⁷ is selected from H and straight chain or branched (C₁-C₆) alkyl (e.g. methyl, ethyl, propyl, butyl, isopropyl, 1-methylpropyl, 2-methylpropyl, t-butyl, pentyl, 3-methylbutyl, 1,2-dimethylpropyl, or 1,1-dimethylbutyl) optionally comprising 1 or 2 double or triple bonds; and
R⁸ is selected from H and straight chain or branched (C₁-C₆) alkyl;
(g) a compound of formula VII 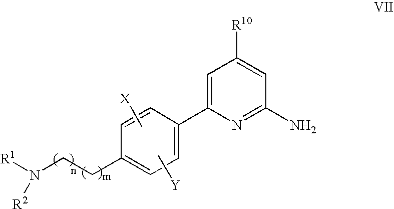
wherein R¹ and R² are selected, independently, from (C₁-C₆) alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₆) alkyl and said tetrahydronaphthalene and the aryl moiety of said aralkyl may optionally be substituted with from one to three substituents, preferably from zero to two substituents, that are selected, independently, from halo (e.g., chloro, fluoro, bromo, iodo), nitro, hydroxy, cyano, amino, (C₁-C₄) alkoxy, and (C₁-C₄) alkylamino;
or R¹ and R² form, together with the nitrogen to which they are attached, a piperazine, piperidine or pyrrolidine ring or an azabicyclic ring containing from 6 to 14 ring members, from 1 to 3 of which are nitrogen and the rest of which are carbon, wherein examples of said azabicyclic rings are the following 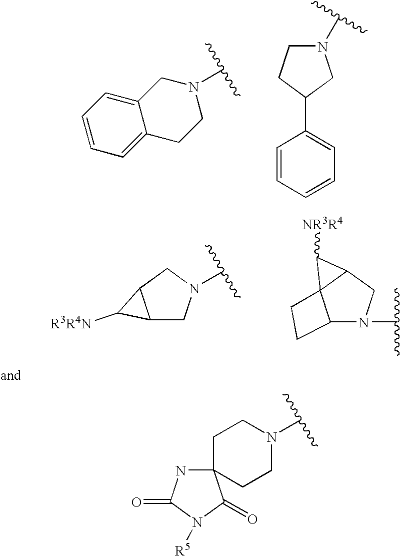
wherein R³ and R⁴ are selected from hydrogen, (C₁-C₆)alkyl, phenyl, naphthyl, (C₁-C₆)alkyl-C(═O)—, HC(═O)—, (C₁-C₆)alkoxy-(C═O)-, phenyl-C(═O)—, naphthyl-C(═O)—, and —(R⁷)₂NC(═O)— wherein each R⁷ is selected, independently, from hydrogen and (C₁-C₆)alkyl;
R⁵ is selected from hydrogen, (C₁-C₆)alkyl, phenyl, napthyl, phenyl-(C₁-C₆)alkyl- and naphthyl (CG-C₆)alkyl-;
and wherein said piperazine, piperidine and pyrorrolidine rings may optionally be substituted with one or more substituents, preferably with from zero to two substituents, that selected independently, from (C₁-C₆) alkylamino, [di(C₁-C₆)alkyl]amino, pheynyl substituted 5 to 6 membered heterocyclic rings containing from 1 to 4 rings nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;
n is 0, 1 or 2; and each carbon of said (CH₂), can optionally be substituted with a substituent R⁸;
m is 0, 1, or 2; and each carbon of said (CH₂)ₘ can optionally be substituted with a substituent R⁹;
(C₁-C₄)alkyl, aryl-(C₁-C₄)alkyl wherein said aryl is selected from phenyl and naphthyl; allyl and phenallyl;
X and Y are selected, independently, from methyl, methoxy, hydroxy and hydrogen; and R¹⁰ is H(C₁-C₆)alkyl;
with the proviso that R⁸ is absent when n is zero and R⁹ is absent when m is zero; and
(h) a compound of formula IX 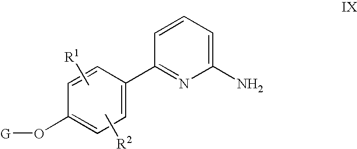
wherein R¹ and R² are selected, independently, from hydrogen, halo, hydroxy, (C₁-C₆)alkoxy, (C₁-C₇)alkyl, (C₂-C₆)alkenyl, and (C₂-C₁₀)alkoxyalkyl; and
G is selected from hydrogen, (C₁-C₆)alkyl, (C₁-C₆)alkoxy-(C₁-C₃)alkyl, aminocarbonyl-(C₁-C₃)alkyl-, (C₁-C₃) alkylaminocarbonyl-(C₁-C₃) alkyl-, di-[(C₁-C₃)alkyl]aminocarbonyl-(C₁-C₃)alkyl-, and N(R³)(R⁴)(C₀-C₄)alkyl-, wherein R³ and R⁴ are selected, independently, from hydrogen, (C₁-C₇) alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₇) alkyl and said tetrahydronaphthalene and the aryl moiety of said aralkyl may optionally be substituted with from one to three substituents, preferably from zero to two substituents, that are selected, independently, from halo, nitro, hydroxy, cyano, amino, (C₁-C₄) alkoxy, and (C₁-C₄) alkylamino;
or R³ and R⁴ form, together with the nitrogen to which they are attached, a piperazine, piperidine, azetidine or pyrrolidine ring or a saturated or unsaturated azabicyclic ring system containing from 6 to 14 ring members, from 1 to 3 of which are nitrogen, from zero to two of which are oxygen, and the rest of which are carbon;
and wherein said piperazine, piperidine, azetidine and pyrrolidine rings and said azabicyclic ring systems may optionally be substituted with one or more substituents, preferably with from zero to two substituents, that are selected, independently, from (C₁-C₆)alkyl, amino, (C₁-C₆) alkylamino, [di-(C₁-C₆)alkyl]amino, phenyl substituted 5 to 6 membered heterocyclic rings containing from 1 to 4 ring nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;
and wherein said piperazine, piperidine, azetidine and pyrrolidine rings and said azabicyclic ring systems may be attached to —(C₀-C₄)alkyl-O— (wherein the oxygen of said —(C₀-C₄)alkyl-O— is the oxygen atom depicted in structural formula I) at a nitrogen atom of the NR³R⁴ ring or at any other atom of such ring having an available bonding site;
or G is a group of the formula A 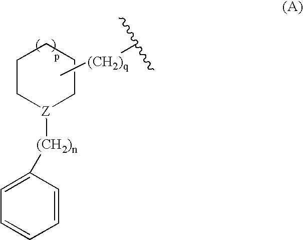
wherein Z is nitrogen or CH, n is zero or one, q is zero, one, two or three and p is zero, one or two;
and wherein the 2-amino piperidine ring depicted in structure I above may optionally be replaced with 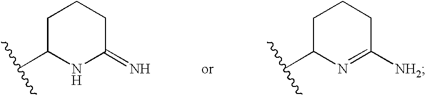
(i) pharmaceutically acceptable salts of said compounds.

20. A method according to claim 18 or 19, wherein the effective amount of the NOS inhibitor is administered to the mammal prior to an event having associated therewith risk of impairment of glucose and/or oxygen to the brain.

21. A method according to claim 18 or 19, wherein the event having associated therewith risk of impairment of glucose and/or oxygen to the brain is an event having associated therewith risk of brain ischemia.

22. A method according to claim 18 or 19, wherein the effective amount of the NOS inhibitor is administered to the mammal prior to a surgery having associated therewith risk of brain ischemia.

23. A method according to claim 22, wherein the surgery is pertaining to the lungs, the cardiovascular system, or the central nervous system, for example the cerebrovascular system.

24. A method according to claim 23, wherein the surgery is cardiac surgery, angioplasty, angiography, or coronary artery bypass graft (CABG).

25. A method according to claim 18 or 19, wherein the effective amount of the NOS inhibitor is administered to the mammal prior to an event wherein hypoxia, anoxia, or asphyxia may be likely to occur.

26. A method according to claim 18 or 19, wherein the mammal to whom the effective amount of the NOS inhibitor is administered is a mammal predisposed to or at risk of brain ischemia, for example predisposed to or at risk of stroke.

27. A method according to claim 26, wherein the mammal has suffered a prior stroke, or has suffered a cardiovascular disease or other condition that impairs the cardiovascular system, for example heart-failure, atrial fibrillation, cardiac ischemia, a hypercoagulative state, birth-control pill use, estrogen replacement therapy, poor circulation, atherosclerosis, or congestive heart failure.
